SEQUENCE LISTING (28 sequences)

SEQ ID NO: 1 - DNA, 2735 bp, Artificial Sequence
  Synthetic
gggaaataagagagaaaagaagagtaagaagaaatataagaccccggcgccgccaccatgggcgcccaggtgagcagacagaacgtgggcacccacagcacccagaacagcgtgagcaacggcagcagcctgaactacttcaacatcaattacttcaaggacgccgcctcatcaggcgccagccggctggacttcagccaggaccccagcaagttcaccgaccccgtgaaggacgtgctggagaagggcatccccaccctgcagagccctagcgtggaggcctgtggctacagcgaccggatcatccagatcacccggggcgactcaaccatcaccagccaggacgtggccaacgccgtggtgggctacggcgtgtggccccattacctgaccccacaggacgccaccgccatcgacaagcccacccagcctgacaccagcagcaaccggttctacaccctggacagcaagatgtggaacagcaccagcaagggctggtggtggaagctgcccgacgctctgaaggacatgggcatcttcggcgagaatatgttctaccactttctggggagaagcggctacaccgtgcacgtgcagtgcaacgccagcaagttccaccagggcaccctgcttgtggtgatgatccccgagcaccagctggccaccgtgaacaagggcaacgtgaacgccggctacaagtacacccatcctggggaggcaggcagagaggtgggcacccaggtggagaacgagaagcagcccagcgacgacaactggctgaacttcgacggcactctgctgggcaacctgctgatctttccccaccagttcatcaacctgcggagcaacaatagcgccaccctgatcgtgccctacgtcaacgccgtacccatggacagcatggtgcggcacaacaactggagcctggtgatcatccccgtgtgccagctgcagagcaacaacatcagcaacatcgtgcccatcaccgtgagcatcagccccatgtgcgccgagttcagcggtgctagagccaagaccgtggtgcagggtctgccagtgtacgtgacaccaggcagcggccagttcatgaccaccgacgacatgcagagcccttgcgcactgccctggtaccaccccaccaaagagatcttcatccccggcgaggtgaagaacctgatcgagatgtgccaggtggacaccctgatccccatcaacagcacccagagcaacatcggcaacgtgagcatgtacaccgtgaccctgagccctcagaccaagctggccgaggagatctttgccatcaaggtggacatcgccagccatcccctggctaccactctgatcggcgagatcgccagctacttcacccactggaccggtagcctgcggttcagcttcatgttctgcggcaccgccaacaccaccctgaaggtgctgctggcctacacgcctcctggcatcgggaagcccaggagcaggaaggaggccatgctgggcacccacgtggtgtgggacgtgggcctgcagagcaccgtgagcctggtggtgccctggatcagcgccagccagtaccggttcaccactcccgacacctacagcagcgccggctacatcacctgctggtaccagaccaacttcgtggtgccacccaacacaccaaacaccgccgagatgctgtgcttcgtgagcggctgcaaccacttctgcctgcggatggccagagacaccgacctgcacaagcagaccggccccatcacccagaaccccgtggagaactacatcgacgaggtgctgaacgaggtcctggtggtgcccaatatcaaccagagccaccccaccacctctaacgccgctcctgtgctggacgctgccgaaaccggacacaccaacaagatccagcccgaggacaccatcgagacccggtacgtgcagagcagccagaccctggacgagatgagcgtggagagctttctgggtaggagcggctgcatccacgagagcgtgctggacatcgtggacaactacaacgaccagagcttcaccaagtggaacatcaacctgcaggagatggcccagatcaggcggaagttcgagatgttcacctacgcccggttcgacagcgagatcaccatggtgcccagcgttgccgctaaggacggccacattggccacatcgtgatgcagtacatgtacgttcctccaggcgcccctatccctaccacccgggacgactacgcttggcagagcggcaccaacgccagcgtgttctggcagcacggacagcccttccctcggttcagcctgcccttcctgagcatcgccagcgcctactacatgttctacgacggatacgacggcgacacctacaagagccggtacggcaccgtggtgaccaacgacatgggcaccctgtgcagccggatcgtgaccagcgagcagctgcacaaggtgaaggtggtgacccggatctaccacaaggccaagcacaccaaggcctggtgtcctagacctccacgtgccgtgcagtacagccacacccacaccaccaactacaagctgagcagcgaggtgcacaacgacgtggccatcagaccccggaccaacctgaccaccgtgtgataataggctggagcctcggtggcctagcttcttgccccttgggcctccccccagcccctcctccccttcctgcacccgtacccccgtggtctttgaataaagtctgagtgggcggc

SEQ ID NO: 2 - DNA, 57 bp, Artificial Sequence
  Synthetic
gggaaataagagagaaaagaagagtaagaagaaatataagaccccggcgccgccacc

SEQ ID NO: 3 - DNA, 2559 bp, Artificial Sequence
  Synthetic
atgggcgcccaggtgagcagacagaacgtgggcacccacagcacccagaacagcgtgagcaacggcagcagcctgaactacttcaacatcaattacttcaaggacgccgcctcatcaggcgccagccggctggacttcagccaggaccccagcaagttcaccgaccccgtgaaggacgtgctggagaagggcatccccaccctgcagagccctagcgtggaggcctgtggctacagcgaccggatcatccagatcacccggggcgactcaaccatcaccagccaggacgtggccaacgccgtggtgggctacggcgtgtggccccattacctgaccccacaggacgccaccgccatcgacaagcccacccagcctgacaccagcagcaaccggttctacaccctggacagcaagatgtggaacagcaccagcaagggctggtggtggaagctgcccgacgctctgaaggacatgggcatcttcggcgagaatatgttctaccactttctggggagaagcggctacaccgtgcacgtgcagtgcaacgccagcaagttccaccagggcaccctgcttgtggtgatgatccccgagcaccagctggccaccgtgaacaagggcaacgtgaacgccggctacaagtacacccatcctggggaggcaggcagagaggtgggcacccaggtggagaacgagaagcagcccagcgacgacaactggctgaacttcgacggcactctgctgggcaacctgctgatctttccccaccagttcatcaacctgcggagcaacaatagcgccaccctgatcgtgccctacgtcaacgccgtacccatggacagcatggtgcggcacaacaactggagcctggtgatcatccccgtgtgccagctgcagagcaacaacatcagcaacatcgtgcccatcaccgtgagcatcagccccatgtgcgccgagttcagcggtgctagagccaagaccgtggtgcagggtctgccagtgtacgtgacaccaggcagcggccagttcatgaccaccgacgacatgcagagcccttgcgcactgccctggtaccaccccaccaaagagatcttcatccccggcgaggtgaagaacctgatcgagatgtgccaggtggacaccctgatccccatcaacagcacccagagcaacatcggcaacgtgagcatgtacaccgtgaccctgagccctcagaccaagctggccgaggagatctttgccatcaaggtggacatcgccagccatcccctggctaccactctgatcggcgagatcgccagctacttcacccactggaccggtagcctgcggttcagcttcatgttctgcggcaccgccaacaccaccctgaaggtgctgctggcctacacgcctcctggcatcgggaagcccaggagcaggaaggaggccatgctgggcacccacgtggtgtgggacgtgggcctgcagagcaccgtgagcctggtggtgccctggatcagcgccagccagtaccggttcaccactcccgacacctacagcagcgccggctacatcacctgctggtaccagaccaacttcgtggtgccacccaacacaccaaacaccgccgagatgctgtgcttcgtgagcggctgcaaccacttctgcctgcggatggccagagacaccgacctgcacaagcagaccggccccatcacccagaaccccgtggagaactacatcgacgaggtgctgaacgaggtcctggtggtgcccaatatcaaccagagccaccccaccacctctaacgccgctcctgtgctggacgctgccgaaaccggacacaccaacaagatccagcccgaggacaccatcgagacccggtacgtgcagagcagccagaccctggacgagatgagcgtggagagctttctgggtaggagcggctgcatccacgagagcgtgctggacatcgtggacaactacaacgaccagagcttcaccaagtggaacatcaacctgcaggagatggcccagatcaggcggaagttcgagatgttcacctacgcccggttcgacagcgagatcaccatggtgcccagcgttgccgctaaggacggccacattggccacatcgtgatgcagtacatgtacgttcctccaggcgcccctatccctaccacccgggacgactacgcttggcagagcggcaccaacgccagcgtgttctggcagcacggacagcccttccctcggttcagcctgcccttcctgagcatcgccagcgcctactacatgttctacgacggatacgacggcgacacctacaagagccggtacggcaccgtggtgaccaacgacatgggcaccctgtgcagccggatcgtgaccagcgagcagctgcacaaggtgaaggtggtgacccggatctaccacaaggccaagcacaccaaggcctggtgtcctagacctccacgtgccgtgcagtacagccacacccacaccaccaactacaagctgagcagcgaggtgcacaacgacgtggccatcagaccccggaccaacctgaccaccgtg

SEQ ID NO: 4 - DNA, 119 bp, Artificial Sequence
  Synthetic
tgataataggctggagcctcggtggcctagcttcttgccccttgggcctccccccagcccctcctccccttcctgcacccgtacccccgtggtctttgaataaagtctgagtgggcggc

SEQ ID NO: 5 - protein, 853 aa, Artificial Sequence
  Synthetic
MGAQVSRQNVGTHSTQNSVSNGSSLNYFNINYFKDAASSGASRLDFSQDPSKFTDPVKDVLEKGIPTLQSPSVEACGYSDRIIQITRGDSTITSQDVANAVVGYGVWPHYLTPQDATAIDKPTQPDTSSNRFYTLDSKMWNSTSKGWWWKLPDALKDMGIFGENMFYHFLGRSGYTVHVQCNASKFHQGTLLVVMIPEHQLATVNKGNVNAGYKYTHPGEAGREVGTQVENEKQPSDDNWLNFDGTLLGNLLIFPHQFINLRSNNSATLIVPYVNAVPMDSMVRHNNWSLVIIPVCQLQSNNISNIVPITVSISPMCAEFSGARAKTVVQGLPVYVTPGSGQFMTTDDMQSPCALPWYHPTKEIFIPGEVKNLIEMCQVDTLIPINSTQSNIGNVSMYTVTLSPQTKLAEEIFAIKVDIASHPLATTLIGEIASYFTHWTGSLRFSFMFCGTANTTLKVLLAYTPPGIGKPRSRKEAMLGTHVVWDVGLQSTVSLVVPWISASQYRFTTPDTYSSAGYITCWYQTNFVVPPNTPNTAEMLCFVSGCNHFCLRMARDTDLHKQTGPITQNPVENYIDEVLNEVLVVPNINQSHPTTSNAAPVLDAAETGHTNKIQPEDTIETRYVQSSQTLDEMSVESFLGRSGCIHESVLDIVDNYNDQSFTKWNINLQEMAQIRRKFEMFTYARFDSEITMVPSVAAKDGHIGHIVMQYMYVPPGAPIPTTRDDYAWQSGTNASVFWQHGQPFPRFSLPFLSIASAYYMFYDGYDGDTYKSRYGTVVTNDMGTLCSRIVTSEQLHKVKVVTRIYHKAKHTKAWCPRPPRAVQYSHTHTTNYKLSSEVHNDVAIRPRTNLTTV

SEQ ID NO: 6 - DNA, 2108 bp, Artificial Sequence
  Synthetic
gggaaataagagagaaaagaagagtaagaagaaatataagaccccggcgccgccaccatgggaccagaagaggaattcggaatgtccatcattaagaacaacacctgtgtggtcaccaccactaacgggaagttcaccggcctgggaatctacgacagaattctcattcttccgactcacgctgacccgggaagcgaaattcaagtcaacggtattcacaccaaggtcctcgattcctacgacctgtttaacaaggaaggggtcaagctggaaatcaccgtgctgaagctggaccggaacgagaagtttcgggatatccgcaagtacatccccgaatccgaggacgactacccggagtgcaacctggcactggtggccaatcaaaccgaacccaccatcatcaaagtcggcgacgtggtgtcctacggaaacatcctgctttccggaactcagacggcccgcatgctgaagtataactacccaaccaagtccgggtactgcggcggtgtcctgtacaagatcggtcagatcctcggaatccacgtcggaggaaacggccgcgacgggttctcctccatgctgttgaggagctacttcactgagcaacaggggcagatccagatcagcaaacacgtgaaggacgtgggccttccctccattcacactcctactaagaccaagctgcagccttccgtgttctacgacattttccccggatccaaggagcccgccgtgctgaccgagaaggatccaagactgaaagtggacttcgactctgccctgttctcaaagtacaagggaaacactgagtgcagccttaacgagcacattcaggtcgcggtggcccactactcggcccaactggctaccctggacatcgaccctcagccgatcgcgatggaagattcagtgttcggaatggacggcctggaagcactggacttgaacacctccgcgggctacccctacgtgaccctcggtatcaagaagaaggacctgattaacaacaagaccaaagatatttcgaagctcaagctcgcgctggataagtacgacgtggacttgccgatgatcaccttcctgaaagacgaactgaggaagaaggataagatcgccgccggcaagactagagtcatcgaagcatccagcattaacgacaccattctgttccggactgtgtacgggaacctgttctcgaagttccatctgaaccccggcgtggtcaccggctgcgccgtgggctgcgacccggaaactttctggagcaagatccctctcatgctggacggagactgtattatggccttcgactataccaactacgacggcagcattcatcctatctggttcaaggccctgggaatggtgctggacaacctgtcgttcaatccgaccctgatcaaccggctgtgtaactccaagcacattttcaagtcgacctactacgaagtggagggcggagtgccgtccggctgctcgggcactagcattttcaactccatgatcaacaatatcattatccgcaccctggtcctcgacgcctacaagcacatcgacctcgataagctcaagatcatcgcctacggcgacgacgtgattttctcatataagtacaaattggacatggaggccatcgctaaggagggacagaaatacggtctgactatcacacccgccgataagagctcagagttcaaggaactcgactacggcaacgtgacgtttctgaagcgcggctttagacaggacgacaagtacaagttcctcatccaccctactttcccggtggaggagatctacgagtccatccggtggactaagaagccgtcccagatgcaggaacacgtgctctccctgtgccacctcatgtggcacaacggccctgagatctataaggactttgagacaaagatacgctctgtgtccgcaggacgggcgctttacatccctccgtacgagctgctgcggcacgagtggtacgagaagttctgataataggctggagcctcggtggcctagcttcttgccccttgggcctccccccagcccctcctccccttcctgcacccgtacccccgtggtctttgaataaagtctgagtgggcggc

SEQ ID NO: 7 - DNA, 1932 bp, Artificial Sequence
  Synthetic
atgggaccagaagaggaattcggaatgtccatcattaagaacaacacctgtgtggtcaccaccactaacgggaagttcaccggcctgggaatctacgacagaattctcattcttccgactcacgctgacccgggaagcgaaattcaagtcaacggtattcacaccaaggtcctcgattcctacgacctgtttaacaaggaaggggtcaagctggaaatcaccgtgctgaagctggaccggaacgagaagtttcgggatatccgcaagtacatccccgaatccgaggacgactacccggagtgcaacctggcactggtggccaatcaaaccgaacccaccatcatcaaagtcggcgacgtggtgtcctacggaaacatcctgctttccggaactcagacggcccgcatgctgaagtataactacccaaccaagtccgggtactgcggcggtgtcctgtacaagatcggtcagatcctcggaatccacgtcggaggaaacggccgcgacgggttctcctccatgctgttgaggagctacttcactgagcaacaggggcagatccagatcagcaaacacgtgaaggacgtgggccttccctccattcacactcctactaagaccaagctgcagccttccgtgttctacgacattttccccggatccaaggagcccgccgtgctgaccgagaaggatccaagactgaaagtggacttcgactctgccctgttctcaaagtacaagggaaacactgagtgcagccttaacgagcacattcaggtcgcggtggcccactactcggcccaactggctaccctggacatcgaccctcagccgatcgcgatggaagattcagtgttcggaatggacggcctggaagcactggacttgaacacctccgcgggctacccctacgtgaccctcggtatcaagaagaaggacctgattaacaacaagaccaaagatatttcgaagctcaagctcgcgctggataagtacgacgtggacttgccgatgatcaccttcctgaaagacgaactgaggaagaaggataagatcgccgccggcaagactagagtcatcgaagcatccagcattaacgacaccattctgttccggactgtgtacgggaacctgttctcgaagttccatctgaaccccggcgtggtcaccggctgcgccgtgggctgcgacccggaaactttctggagcaagatccctctcatgctggacggagactgtattatggccttcgactataccaactacgacggcagcattcatcctatctggttcaaggccctgggaatggtgctggacaacctgtcgttcaatccgaccctgatcaaccggctgtgtaactccaagcacattttcaagtcgacctactacgaagtggagggcggagtgccgtccggctgctcgggcactagcattttcaactccatgatcaacaatatcattatccgcaccctggtcctcgacgcctacaagcacatcgacctcgataagctcaagatcatcgcctacggcgacgacgtgattttctcatataagtacaaattggacatggaggccatcgctaaggagggacagaaatacggtctgactatcacacccgccgataagagctcagagttcaaggaactcgactacggcaacgtgacgtttctgaagcgcggctttagacaggacgacaagtacaagttcctcatccaccctactttcccggtggaggagatctacgagtccatccggtggactaagaagccgtcccagatgcaggaacacgtgctctccctgtgccacctcatgtggcacaacggccctgagatctataaggactttgagacaaagatacgctctgtgtccgcaggacgggcgctttacatccctccgtacgagctgctgcggcacgagtggtacgagaagttc

SEQ ID NO: 8 - protein, 644 aa, Artificial Sequence
  Synthetic
MGPEEEFGMSIIKNNTCVVTTTNGKFTGLGIYDRILILPTHADPGSEIQVNGIHTKVLDSYDLFNKEGVKLEITVLKLDRNEKFRDIRKYIPESEDDYPECNLALVANQTEPTIIKVGDVVSYGNILLSGTQTARMLKYNYPTKSGYCGGVLYKIGQILGIHVGGNGRDGFSSMLLRSYFTEQQGQIQISKHVKDVGLPSIHTPTKTKLQPSVFYDIFPGSKEPAVLTEKDPRLKVDFDSALFSKYKGNTECSLNEHIQVAVAHYSAQLATLDIDPQPIAMEDSVFGMDGLEALDLNTSAGYPYVTLGIKKKDLINNKTKDISKLKLALDKYDVDLPMITFLKDELRKKDKIAAGKTRVIEASSINDTILFRTVYGNLFSKFHLNPGVVTGCAVGCDPETFWSKIPLMLDGDCIMAFDYTNYDGSIHPIWFKALGMVLDNLSFNPTLINRLCNSKHIFKSTYYEVEGGVPSGCSGTSIFNSMINNIIIRTLVLDAYKHIDLDKLKIIAYGDDVIFSYKYKLDMEAIAKEGQKYGLTITPADKSSEFKELDYGNVTFLKRGFRQDDKYKFLIHPTFPVEEIYESIRWTKKPSQMQEHVLSLCHLMWHNGPEIYKDFETKIRSVSAGRALYIPPYELLRHEWYEKF

SEQ ID NO: 9 - DNA, 2744 bp, Artificial Sequence
  Synthetic
gggaaataagagagaaaagaagagtaagaagaaatataagaccccggcgccgccaccatgggcgcccaggtgagcacccagaagtcgggcagccacgagaaccagaacatcctgaccaacggcagcaaccagaccttcaccgtgatcaactactacaaggacgccgccagcaccagcagcgccggccagagcctgagcatggaccctagcaagttcaccgagcctgtgaaggacctgatgctgaagggcgcccctgccctgaacagccctaacgtggaggcctgcggctacagcgacagagtgcagcagatcaccctgggcaacagcaccatcaccacccaggaggccgccaacgccgtggtgtgctacgccgagtggcctgagtacctgcctgacgtggacgccagcgacgtgaacaagaccagcaagcctgacaccagcgtgtgcagattctacaccctggacagcaagacctggaccactggctccaagggctggtgctggaagcttcctgacgccctcaaggacatgggcgtgttcggacagaatatgttcttccacagcctgggcagaagcggttacaccgtgcacgtgcagtgcaacgccaccaagttccattccggctgcctgctggtggtggtgatccctgagcaccaactggcttcgcacgaaggcggcaacgtgagcgtgaagtacacgttcactcacccaggcgagagaggcatcgacctgtcaagcgcaaacgaggtgggcggccctgttaaggacgtgatctacaacatgaacggcaccctgttaggtaatctgctgatcttccctcaccagttcatcaacctgagaaccaacaacaccgccaccatcgtcattccatacatcaacagcgtgcctatcgacagcatgaccagacacaacaacgtcagtctgatggtaatcccaatcgcccctctgaccgtgcctaccggcgccacccctagcctgcctataaccgtgaccatcgctcctatgtgcaccgagttcagcggcatcagaagcaagagcatcgtgcctcagggtctaccgaccaccaccctgcctggcagcggccagttcctaactaccgacgacagacagagccctagcgccctgccaaattacgagcctacccctagaatccacatccctggcaaggtgcacaacctgctggagatcatccaggtggacaccttgatcccgatgaataatacccacaccaaggacgaggtgaacagctatctgataccgctgaacgccaataggcagaacgagcaggtgttcggcaccaacctgttcatcggcgacggagtgttcaagacaaccctgctgggagagatcgtgcagtactacacccactggtctggaagtctgagattcagtcttatgtacaccggtcctgcactaagctctgccaagctgatcctggcctacacccctcctggcgccagaggccctcaggacagaagagaggccatgctaggcacccacgtcgtttgggacatcggcctgcagtcaacaattgtaatgaccatcccttggaccagcggcgtgcagttcagatacaccgaccctgatacttacacatccgcaggcttcttgagctgctggtatcagacctcactgatccttcctcctgagacaactggccaggtgtatctgctatcgttcatcagcgcttgccctgacttcaagctgagactgatgaaggatacccagaccatcagccagaccgtggccctgaccgagggcctgggcgacgagctggaggaggtgatcgtggagaagaccaagcagacggtggccagcatctctagcggacctaagcatacacagaaggtcccaatcttgactgctaacgagacaggagccactatgcctgtgctgccttccgacagtatcgaaacaagaaccacctacatgcacttcaacggatctgaaacagacgtggagtgcttccttggacgagccgcctgcgtccacgtaaccgagatccagaacaaggacgccaccggaattgacaaccacagggaggcaaagttgttcaacgactggaagattaacttgagtagcctggtgcagctgagaaagaagctggagttgttcacctacgtgagattcgacagcgagtacaccattctggcaaccgcctcccagccagattccgccaactactcttccaacctggttgtgcaggccatgtacgtaccgccgggagccccgaaccctaaggagtgggacgactacacctggcagagcgccagcaaccctagcgtgttcttcaaggttggcgatacctccaggttcagtgtcccatacgtgggcctggcgtccgcctacaactgcttctacgacggatacagccacgacgacgccgaaacacagtacggcatcaccgtactgaaccacatgggcagcatggccttcagaatcgttaacgaacacgacgagcacaagaccctggtgaagatcagagtgtatcacagagccaagcacgtcgaggcctggatccctagagcaccacgggcactaccatatactagcatcggcagaaccaattaccctaagaacacagaaccagtgatcaagaagagaaagggcgacatcaagagctactgataataggctggagcctcggtggcctagcttcttgccccttgggcctccccccagcccctcctccccttcctgcacccgtacccccgtggtctttgaataaagtctgagtgggcggc

SEQ ID NO: 10 - DNA, 2568 bp, Artificial Sequence
  Synthetic
atgggcgcccaggtgagcacccagaagtcgggcagccacgagaaccagaacatcctgaccaacggcagcaaccagaccttcaccgtgatcaactactacaaggacgccgccagcaccagcagcgccggccagagcctgagcatggaccctagcaagttcaccgagcctgtgaaggacctgatgctgaagggcgcccctgccctgaacagccctaacgtggaggcctgcggctacagcgacagagtgcagcagatcaccctgggcaacagcaccatcaccacccaggaggccgccaacgccgtggtgtgctacgccgagtggcctgagtacctgcctgacgtggacgccagcgacgtgaacaagaccagcaagcctgacaccagcgtgtgcagattctacaccctggacagcaagacctggaccactggctccaagggctggtgctggaagcttcctgacgccctcaaggacatgggcgtgttcggacagaatatgttcttccacagcctgggcagaagcggttacaccgtgcacgtgcagtgcaacgccaccaagttccattccggctgcctgctggtggtggtgatccctgagcaccaactggcttcgcacgaaggcggcaacgtgagcgtgaagtacacgttcactcacccaggcgagagaggcatcgacctgtcaagcgcaaacgaggtgggcggccctgttaaggacgtgatctacaacatgaacggcaccctgttaggtaatctgctgatcttccctcaccagttcatcaacctgagaaccaacaacaccgccaccatcgtcattccatacatcaacagcgtgcctatcgacagcatgaccagacacaacaacgtcagtctgatggtaatcccaatcgcccctctgaccgtgcctaccggcgccacccctagcctgcctataaccgtgaccatcgctcctatgtgcaccgagttcagcggcatcagaagcaagagcatcgtgcctcagggtctaccgaccaccaccctgcctggcagcggccagttcctaactaccgacgacagacagagccctagcgccctgccaaattacgagcctacccctagaatccacatccctggcaaggtgcacaacctgctggagatcatccaggtggacaccttgatcccgatgaataatacccacaccaaggacgaggtgaacagctatctgataccgctgaacgccaataggcagaacgagcaggtgttcggcaccaacctgttcatcggcgacggagtgttcaagacaaccctgctgggagagatcgtgcagtactacacccactggtctggaagtctgagattcagtcttatgtacaccggtcctgcactaagctctgccaagctgatcctggcctacacccctcctggcgccagaggccctcaggacagaagagaggccatgctaggcacccacgtcgtttgggacatcggcctgcagtcaacaattgtaatgaccatcccttggaccagcggcgtgcagttcagatacaccgaccctgatacttacacatccgcaggcttcttgagctgctggtatcagacctcactgatccttcctcctgagacaactggccaggtgtatctgctatcgttcatcagcgcttgccctgacttcaagctgagactgatgaaggatacccagaccatcagccagaccgtggccctgaccgagggcctgggcgacgagctggaggaggtgatcgtggagaagaccaagcagacggtggccagcatctctagcggacctaagcatacacagaaggtcccaatcttgactgctaacgagacaggagccactatgcctgtgctgccttccgacagtatcgaaacaagaaccacctacatgcacttcaacggatctgaaacagacgtggagtgcttccttggacgagccgcctgcgtccacgtaaccgagatccagaacaaggacgccaccggaattgacaaccacagggaggcaaagttgttcaacgactggaagattaacttgagtagcctggtgcagctgagaaagaagctggagttgttcacctacgtgagattcgacagcgagtacaccattctggcaaccgcctcccagccagattccgccaactactcttccaacctggttgtgcaggccatgtacgtaccgccgggagccccgaaccctaaggagtgggacgactacacctggcagagcgccagcaaccctagcgtgttcttcaaggttggcgatacctccaggttcagtgtcccatacgtgggcctggcgtccgcctacaactgcttctacgacggatacagccacgacgacgccgaaacacagtacggcatcaccgtactgaaccacatgggcagcatggccttcagaatcgttaacgaacacgacgagcacaagaccctggtgaagatcagagtgtatcacagagccaagcacgtcgaggcctggatccctagagcaccacgggcactaccatatactagcatcggcagaaccaattaccctaagaacacagaaccagtgatcaagaagagaaagggcgacatcaagagctac

SEQ ID NO: 11 - protein, 856 aa, Artificial Sequence
  Synthetic
MGAQVSTQKSGSHENQNILTNGSNQTFTVINYYKDAASTSSAGQSLSMDPSKFTEPVKDLMLKGAPALNSPNVEACGYSDRVQQITLGNSTITTQEAANAVVCYAEWPEYLPDVDASDVNKTSKPDTSVCRFYTLDSKTWTTGSKGWCWKLPDALKDMGVFGQNMFFHSLGRSGYTVHVQCNATKFHSGCLLVVVIPEHQLASHEGGNVSVKYTFTHPGERGIDLSSANEVGGPVKDVIYNMNGTLLGNLLIFPHQFINLRTNNTATIVIPYINSVPIDSMTRHNNVSLMVIPIAPLTVPTGATPSLPITVTIAPMCTEFSGIRSKSIVPQGLPTTTLPGSGQFLTTDDRQSPSALPNYEPTPRIHIPGKVHNLLEIIQVDTLIPMNNTHTKDEVNSYLIPLNANRQNEQVFGTNLFIGDGVFKTTLLGEIVQYYTHWSGSLRFSLMYTGPALSSAKLILAYTPPGARGPQDRREAMLGTHVVWDIGLQSTIVMTIPWTSGVQFRYTDPDTYTSAGFLSCWYQTSLILPPETTGQVYLLSFISACPDFKLRLMKDTQTISQTVALTEGLGDELEEVIVEKTKQTVASISSGPKHTQKVPILTANETGATMPVLPSDSIETRTTYMHFNGSETDVECFLGRAACVHVTEIQNKDATGIDNHREAKLFNDWKINLSSLVQLRKKLELFTYVRFDSEYTILATASQPDSANYSSNLVVQAMYVPPGAPNPKEWDDYTWQSASNPSVFFKVGDTSRFSVPYVGLASAYNCFYDGYSHDDAETQYGITVLNHMGSMAFRIVNEHDEHKTLVKIRVYHRAKHVEAWIPRAPRALPYTSIGRTNYPKNTEPVIKKRKGDIKSY

SEQ ID NO: 12 - DNA, 2105 bp, Artificial Sequence
  Synthetic
gggaaataagagagaaaagaagagtaagaagaaatataagaccccggcgccgccaccatgggccctaacaccgagttcgccctgagcctgctgagaaagaacatcatgaccatcaccaccagcaagggcgagttcaccggcctgggcatccacgacagagtgtgcgtgatccctacccacgcccagcctggcgacgacgtgctggtgaacggccagaagatcagagtgaaggacaagtacaagctggtggaccctgagaacattaacctggagctgaccgtgctgaccctggacagaaacgagaagttcagagacatcagaggcttcatcagcgaggacctggagggcgtggacgccaccctggtggtgcacagcaacaacttcaccaacaccatcctggaggtgggccctgtgaccatggccggcctgattaatttgagcagcacccctaccaacagaatgatcagatacgactacgccaccaagaccggccagtgcggcggcgtgctgtgcgccaccggcaagatcttcggtatacacgtgggcggcaacggcagacagggcttcagcgcccagctgaagaagcagtacttcgtggagaagcagggccaggtgatcgccagacacaaggtgagagagttcaatatcaatcctgtgaacacaccaactaagagcaagctgcaccctagcgtgttctacgacgtgttcccaggagacaaggagcctgcagtcctgagcgacaacgaccctagacttgaagtcaagcttactgagagcctgttctctaagtataagggcaacgtcaacacagagcctaccgagaacatgctggtggccgtggaccactacgccggccagctgctatctctggacatcccaacaagcgagctgacgttgaaggaggccctgtacggcgtcgacggcctggagcctatcgatatcacgaccagcgccggcttcccttacgtgtccctgggtatcaagaagagggatattctgaacaaggagactcaggacaccgagaagatgaagttctacctcgataagtacggcatcgacctgcctctggtgacctacatcaaggacgagctgagaagcgtggacaaggtccgcttgggcaagagcagactgatcgaggccagcagcctgaacgacagcgtgaacatgagaatgaagctgggcaacctgtacaaggccttccaccagaaccctggagtattgaccggcagcgccgtgggctgcgaccctgacgtgttctggagcgttatcccttgcctgatggacggacacctgatggccttcgactacagcaacttcgacgccagcctgagccctgtgtggttcgtgtgcctggagaaggtcctgaccaagctgggtttcgccggttcctctttgatccagagcatctgcaacacccaccacatcttccgggacgagatctacgtggtagagggtggcatgcctagcggctgcagcggcaccagcatcttcaacagcatgatcaacaacatcatcattagaacactgatcctggacgcctataagggaattgacctggataagttgaagatcctggcctacggagacgacctgatcgtgagctacccttacgagctggaccctcaggtgctggcaaccctcggaaagaactacggcttgacaatcacgcctcctgacaagagcgagacattcacaaagatgacttgggagaacctgaccttcctgaagagatacttcaagcctgaccagcagttccctttcctggtgcaccctgtcatgcctatgaaggatattcacgagagcatccggtggaccaaggaccctaagaacactcaggaccacgtgagaagcctgtgcatgctggcctggcacagcggcgagaaggagtacaacgagttcattcagaagattaggactaccgacatcggcaagtgcctgattcttcctgagtactcagtgcttaggaggcgctggctggacctgttctgataataggctggagcctcggtggcctagcttcttgccccttgggcctccccccagcccctcctccccttcctgcacccgtacccccgtggtctttgaataaagtctgagtgggcggc

SEQ ID NO: 13 - DNA, 1929 bp, Artificial Sequence
  Synthetic
atgggccctaacaccgagttcgccctgagcctgctgagaaagaacatcatgaccatcaccaccagcaagggcgagttcaccggcctgggcatccacgacagagtgtgcgtgatccctacccacgcccagcctggcgacgacgtgctggtgaacggccagaagatcagagtgaaggacaagtacaagctggtggaccctgagaacattaacctggagctgaccgtgctgaccctggacagaaacgagaagttcagagacatcagaggcttcatcagcgaggacctggagggcgtggacgccaccctggtggtgcacagcaacaacttcaccaacaccatcctggaggtgggccctgtgaccatggccggcctgattaatttgagcagcacccctaccaacagaatgatcagatacgactacgccaccaagaccggccagtgcggcggcgtgctgtgcgccaccggcaagatcttcggtatacacgtgggcggcaacggcagacagggcttcagcgcccagctgaagaagcagtacttcgtggagaagcagggccaggtgatcgccagacacaaggtgagagagttcaatatcaatcctgtgaacacaccaactaagagcaagctgcaccctagcgtgttctacgacgtgttcccaggagacaaggagcctgcagtcctgagcgacaacgaccctagacttgaagtcaagcttactgagagcctgttctctaagtataagggcaacgtcaacacagagcctaccgagaacatgctggtggccgtggaccactacgccggccagctgctatctctggacatcccaacaagcgagctgacgttgaaggaggccctgtacggcgtcgacggcctggagcctatcgatatcacgaccagcgccggcttcccttacgtgtccctgggtatcaagaagagggatattctgaacaaggagactcaggacaccgagaagatgaagttctacctcgataagtacggcatcgacctgcctctggtgacctacatcaaggacgagctgagaagcgtggacaaggtccgcttgggcaagagcagactgatcgaggccagcagcctgaacgacagcgtgaacatgagaatgaagctgggcaacctgtacaaggccttccaccagaaccctggagtattgaccggcagcgccgtgggctgcgaccctgacgtgttctggagcgttatcccttgcctgatggacggacacctgatggccttcgactacagcaacttcgacgccagcctgagccctgtgtggttcgtgtgcctggagaaggtcctgaccaagctgggtttcgccggttcctctttgatccagagcatctgcaacacccaccacatcttccgggacgagatctacgtggtagagggtggcatgcctagcggctgcagcggcaccagcatcttcaacagcatgatcaacaacatcatcattagaacactgatcctggacgcctataagggaattgacctggataagttgaagatcctggcctacggagacgacctgatcgtgagctacccttacgagctggaccctcaggtgctggcaaccctcggaaagaactacggcttgacaatcacgcctcctgacaagagcgagacattcacaaagatgacttgggagaacctgaccttcctgaagagatacttcaagcctgaccagcagttccctttcctggtgcaccctgtcatgcctatgaaggatattcacgagagcatccggtggaccaaggaccctaagaacactcaggaccacgtgagaagcctgtgcatgctggcctggcacagcggcgagaaggagtacaacgagttcattcagaagattaggactaccgacatcggcaagtgcctgattcttcctgagtactcagtgcttaggaggcgctggctggacctgttc

SEQ ID NO: 14 - protein, 643 aa, Artificial Sequence
  Synthetic
MGPNTEFALSLLRKNIMTITTSKGEFTGLGIHDRVCVIPTHAQPGDDVLVNGQKIRVKDKYKLVDPENINLELTVLTLDRNEKFRDIRGFISEDLEGVDATLVVHSNNFTNTILEVGPVTMAGLINLSSTPTNRMIRYDYATKTGQCGGVLCATGKIFGIHVGGNGRQGFSAQLKKQYFVEKQGQVIARHKVREFNINPVNTPTKSKLHPSVFYDVFPGDKEPAVLSDNDPRLEVKLTESLFSKYKGNVNTEPTENMLVAVDHYAGQLLSLDIPTSELTLKEALYGVDGLEPIDITTSAGFPYVSLGIKKRDILNKETQDTEKMKFYLDKYGIDLPLVTYIKDELRSVDKVRLGKSRLIEASSLNDSVNMRMKLGNLYKAFHQNPGVLTGSAVGCDPDVFWSVIPCLMDGHLMAFDYSNFDASLSPVWFVCLEKVLTKLGFAGSSLIQSICNTHHIFRDEIYVVEGGMPSGCSGTSIFNSMINNIIIRTLILDAYKGIDLDKLKILAYGDDLIVSYPYELDPQVLATLGKNYGLTITPPDKSETFTKMTWENLTFLKRYFKPDQQFPFLVHPVMPMKDIHESIRWTKDPKNTQDHVRSLCMLAWHSGEKEYNEFIQKIRTTDIGKCLILPEYSVLRRRWLDLF

SEQ ID NO: 15 - DNA, 2759 bp, Artificial Sequence
  Synthetic
gggaaataagagagaaaagaagagtaagaagaaatataagaccccggcgccgccaccatgggcgcccaggtgacgcgccagcagaccggcacccacgagaacgccaacatcgccaccaacggcagccacatcacctataaccagatcaacttctataaggactcttacgcagccagcgccagcaagcaggactttagccaggacccttcaaagttcaccgagcctgtggtggagggcctgaaggccggcgcacccgtgctgaagtcccctagcgcagaggcctgcggatacagcgacagagtgcttcagctgaaactgggcaatagcgctatcgtgacccaggaagcggccaactactgttgcgcctacggtgagtggcctaactatctgccagaccacgaggccgtggcgatcgataaacctactcagcctgagacagcgacagacagattctacaccttaaagagtgtgaagtgggaaaccggcagcaccggctggtggtggaagctacctgacgccctgaacaacatcggcatgttcggccagaacgtgcagcatcactacctctaccggagcggcttcctgattcacgtgcagtgcaacgccaccaagtttcaccagggagccctgctggtcgtagcaatccctgagcatcaaagaggcgcccacaacaccaacaccagccctggctttgacgacatcatgaagggcgaagagggcggcacattcaaccacccttacgtgctggacgacggcaccagcctggcctgcgcgaccatcttccctcaccagtggatcaacctcagaaccaacaactctgctaccatcgtgctgccttggatgaacgcggcccctatggacttccctctgagacacaatcagtggaccctcgccattatccctgtggtgcccctgggcaccagaaccaccagcagcatggtgcctatcaccgtcagcattgccccaatgtgctgcgaattcaacggcttgcggcacgccatcacacagggcgtgcccacctacctgctgcccggtagcggccagtttctgaccaccgacgaccactcgtctgcacctgctctcccttgcttcaatcctaccccagagatgcacatccctggccaggtgagaaacatgctggaggtggtgcaggtggagagcatgatggaaatcaacaacaccgaaagcgccgtcggcatggagcgcctgaaagtggacatctccgcgctgactgacgtggaccagctgctgttcaacatccctctggacatccagctggacgggcctcttcgcaacaccctggttggcaacatcagcagatactacactcactggagtggcagcctggagatgaccttcatgttctgcggaagtttcatggccaccggcaagctgatcctttgctacactcctcctggcggcagctgtcctaccacacgggagactgccatgctgggaacccacatcgtgtgggacttcgggctgcagagcagcgtgaccctgatcatcccttggatcagcggctctcactacagaatgtttaacaacgacgctaagagcaccaacgccaacgtgggctacgtgacctgcttcatgcagacaaacctgatagtgcctagcgagtcaagcgacacctgcagcctgatcgggttcatcgccgccaaagacgactttagccttagactgatgcgagacagccctgatatcgggcagcttgaccacctgcacgccgccgaggcagcttaccaaatcgagagcattatcaagaccgccaccgataccgtcaagagtgagatcaacgccgaactgggcgttgtgcctagcctgaacgccgtggagacaggcgccaccagcaacaccgagcctgaggaggccatacagaccagaaccgtgatcaatcagcacggcgtgagcgagacactggtcgagaacttcttgagcagagctgccctcgtgagcaagagatctttcgagtacaaggaccacacctcttccgccgcccaagccgacaagaacttcttcaagtggaccatcaacactaggagcttcgtgcagctaagaaggaagctggagctgtttacttacctgagattcgacgctgagatcaccattctgacgaccgtggccgtgaacggcagcagcaataacacatacgtcggactgcctgatctgacgctgcaggccatgttcgtgcctaccggcgccctgacacctgagaagcaagacagcttccactggcagagcggcagcaacgcaagcgtgttcttcaagatcagcgatcctcctgcacgcatcaccatcccattcatgtgcatcaatagcgcctattccgtgttctacgacggcttcgccggcttcgagaagaacggcctttacggcatcaatcccgccgacactatcggcaacctgtgcgtgagaatcgtgaacgagcaccagcctgtcgggttcactgtgaccgttagagtgtacatgaagcctaagcacatcaaggcctgggcccctcgtccacctagaaccctgccttacatgtctattgctaacgccaactacaagggcaaggagagagcccctaacgcgctctcagccatcattggcaatcgtgacagcgtgaagaccatgcctcacaacattgtgaacacctgataataggctggagcctcggtggcctagcttcttgccccttgggcctccccccagcccctcctccccttcctgcacccgtacccccgtggtctttgaataaagtctgagtgggcggc

SEQ ID NO: 16 - DNA, 2583 bp, Artificial Sequence
  Synthetic
atgggcgcccaggtgacgcgccagcagaccggcacccacgagaacgccaacatcgccaccaacggcagccacatcacctataaccagatcaacttctataaggactcttacgcagccagcgccagcaagcaggactttagccaggacccttcaaagttcaccgagcctgtggtggagggcctgaaggccggcgcacccgtgctgaagtcccctagcgcagaggcctgcggatacagcgacagagtgcttcagctgaaactgggcaatagcgctatcgtgacccaggaagcggccaactactgttgcgcctacggtgagtggcctaactatctgccagaccacgaggccgtggcgatcgataaacctactcagcctgagacagcgacagacagattctacaccttaaagagtgtgaagtgggaaaccggcagcaccggctggtggtggaagctacctgacgccctgaacaacatcggcatgttcggccagaacgtgcagcatcactacctctaccggagcggcttcctgattcacgtgcagtgcaacgccaccaagtttcaccagggagccctgctggtcgtagcaatccctgagcatcaaagaggcgcccacaacaccaacaccagccctggctttgacgacatcatgaagggcgaagagggcggcacattcaaccacccttacgtgctggacgacggcaccagcctggcctgcgcgaccatcttccctcaccagtggatcaacctcagaaccaacaactctgctaccatcgtgctgccttggatgaacgcggcccctatggacttccctctgagacacaatcagtggaccctcgccattatccctgtggtgcccctgggcaccagaaccaccagcagcatggtgcctatcaccgtcagcattgccccaatgtgctgcgaattcaacggcttgcggcacgccatcacacagggcgtgcccacctacctgctgcccggtagcggccagtttctgaccaccgacgaccactcgtctgcacctgctctcccttgcttcaatcctaccccagagatgcacatccctggccaggtgagaaacatgctggaggtggtgcaggtggagagcatgatggaaatcaacaacaccgaaagcgccgtcggcatggagcgcctgaaagtggacatctccgcgctgactgacgtggaccagctgctgttcaacatccctctggacatccagctggacgggcctcttcgcaacaccctggttggcaacatcagcagatactacactcactggagtggcagcctggagatgaccttcatgttctgcggaagtttcatggccaccggcaagctgatcctttgctacactcctcctggcggcagctgtcctaccacacgggagactgccatgctgggaacccacatcgtgtgggacttcgggctgcagagcagcgtgaccctgatcatcccttggatcagcggctctcactacagaatgtttaacaacgacgctaagagcaccaacgccaacgtgggctacgtgacctgcttcatgcagacaaacctgatagtgcctagcgagtcaagcgacacctgcagcctgatcgggttcatcgccgccaaagacgactttagccttagactgatgcgagacagccctgatatcgggcagcttgaccacctgcacgccgccgaggcagcttaccaaatcgagagcattatcaagaccgccaccgataccgtcaagagtgagatcaacgccgaactgggcgttgtgcctagcctgaacgccgtggagacaggcgccaccagcaacaccgagcctgaggaggccatacagaccagaaccgtgatcaatcagcacggcgtgagcgagacactggtcgagaacttcttgagcagagctgccctcgtgagcaagagatctttcgagtacaaggaccacacctcttccgccgcccaagccgacaagaacttcttcaagtggaccatcaacactaggagcttcgtgcagctaagaaggaagctggagctgtttacttacctgagattcgacgctgagatcaccattctgacgaccgtggccgtgaacggcagcagcaataacacatacgtcggactgcctgatctgacgctgcaggccatgttcgtgcctaccggcgccctgacacctgagaagcaagacagcttccactggcagagcggcagcaacgcaagcgtgttcttcaagatcagcgatcctcctgcacgcatcaccatcccattcatgtgcatcaatagcgcctattccgtgttctacgacggcttcgccggcttcgagaagaacggcctttacggcatcaatcccgccgacactatcggcaacctgtgcgtgagaatcgtgaacgagcaccagcctgtcgggttcactgtgaccgttagagtgtacatgaagcctaagcacatcaaggcctgggcccctcgtccacctagaaccctgccttacatgtctattgctaacgccaactacaagggcaaggagagagcccctaacgcgctctcagccatcattggcaatcgtgacagcgtgaagaccatgcctcacaacattgtgaacacc

SEQ ID NO: 17 - protein, 861 aa, Artificial Sequence
  Synthetic
MGAQVTRQQTGTHENANIATNGSHITYNQINFYKDSYAASASKQDFSQDPSKFTEPVVEGLKAGAPVLKSPSAEACGYSDRVLQLKLGNSAIVTQEAANYCCAYGEWPNYLPDHEAVAIDKPTQPETATDRFYTLKSVKWETGSTGWWWKLPDALNNIGMFGQNVQHHYLYRSGFLIHVQCNATKFHQGALLVVAIPEHQRGAHNTNTSPGFDDIMKGEEGGTFNHPYVLDDGTSLACATIFPHQWINLRTNNSATIVLPWMNAAPMDFPLRHNQWTLAIIPVVPLGTRTTSSMVPITVSIAPMCCEFNGLRHAITQGVPTYLLPGSGQFLTTDDHSSAPALPCFNPTPEMHIPGQVRNMLEVVQVESMMEINNTESAVGMERLKVDISALTDVDQLLFNIPLDIQLDGPLRNTLVGNISRYYTHWSGSLEMTFMFCGSFMATGKLILCYTPPGGSCPTTRETAMLGTHIVWDFGLQSSVTLIIPWISGSHYRMFNNDAKSTNANVGYVTCFMQTNLIVPSESSDTCSLIGFIAAKDDFSLRLMRDSPDIGQLDHLHAAEAAYQIESIIKTATDTVKSEINAELGVVPSLNAVETGATSNTEPEEAIQTRTVINQHGVSETLVENFLSRAALVSKRSFEYKDHTSSAAQADKNFFKWTINTRSFVQLRRKLELFTYLRFDAEITILTTVAVNGSSNNTYVGLPDLTLQAMFVPTGALTPEKQDSFHWQSGSNASVFFKISDPPARITIPFMCINSAYSVFYDGFAGFEKNGLYGINPADTIGNLCVRIVNEHQPVGFTVTVRVYMKPKHIKAWAPRPPRTLPYMSIANANYKGKERAPNALSAIIGNRDSVKTMPHNIVNT

SEQ ID NO: 18 - DNA, 2099 bp, Artificial Sequence
  Synthetic
gggaaataagagagaaaagaagagtaagaagaaatataagaccccggcgccgccaccatgggccctggcttcgacttcgcccaggccatcatgaagaagaacaccgtgatcgccagaaccgagaagggcgagttcaccatgctgggcgtgtacgacagagtggccgtgatccctacccacgccagcgtgggcgaaaccatctacatcaacgacgtggaaacgaaggtgctggacgcctgcgccctgcgcgacctgaccgacaccaacctggagatcaccatcgtgaagctggacagaaaccagaagttcagagacatcagacacttcctgcctagatacgaggacgactacaacgacgccgtgctgagcgtgcacaccagcaagttccctaacatgtacatccctgtgggccaggtgaccaactacggcttcctgaacctgggcggcaccccaactcatagaatcctgatgtacaacttccctaccagagccggccagtgcggcggcgtggtgaccaccaccggcaaggtgatcggcatccacgtgggcggaaacggcgcccagggcttcgccgccatgctgctgcacagctacttcaccgatacccagggcgagatcgtgagcagcgagaagtccggcgtgtgcatcaacgcccctgccaagaccaagctgcagcctagcgtgttccaccaggtgttcgagggcagcaaggagcctgcagtactgaaccctaaggaccctagactgaagaccgacttcgaggaggccatcttcagcaagtatactggtaacaagatcatgctgatggacgagtacatggaagaggcggtggaccactacgtgggctgcctggagcctctggacatcagcgtggaccctatccctctggagagcgccatgtacggcatggacggcctggaggccctggacttgactactagcgccggcttcccttacctgctgcagggcaagaagaagcgagacatcttcaacagacacaccagagacaccagcgagatgaccaagatgctggagaagtacggtgtggatctgcctttcgtgaccttcgtgaaggacgagctgagaagcagagagaaggtggagaagggtaagagcagactgatcgaggccagcagcctgaacgacagcgtggccatgagagtggctttcggcaacctgtacgccaccttccacaacaaccctggcaccgctacgggtagcgccgtgggttgcgaccctgatatcttctggagcaagatccctatcttactggacggagagatattcgccttcgactacaccggctacgacgccagcctgagccctgtgtggttcgcctgcctgaagaaggtgttgatcaagctgggctacacccaccagaccagcttcatcgactacctgtgccactcggtgcatctgtacaaggacaagaagtatatcgtcaacggcggcatgcctagcggcagcagcggcacctctatcttcaataccatgatcaacaacatcatcatcagaaccctgctcattagagtctataagggcatcgacctggaccagttcaagatgatcgcctacggcgacgacgtaatcgccagctaccctcacaagatcgaccctggcctgctggccgaggccggcaagcagtacggactggtgatgacccctgccgacaagggaacttcgttcattgatactaattgggagaacgtcaccttccttaagagatacttcagagccgacgaccagtaccctttcctgatccaccctgtgatgcctatgaaggagatccacgagagcatccgctggaccaaggatccacgcaacacccaggaccacgtgagaagcctgtgctatctggcttggcacaacggcgaagaagcctataacgagttctgcagaaagatcagaagcgtgccagtgggcagggccctgaccctgcctgcctactcgtcgttgagaagaaagtggttggacagcttctgataataggctggagcctcggtggcctagcttcttgccccttgggcctccccccagcccctcctccccttcctgcacccgtacccccgtggtctttgaataaagtctgagtgggcggc

SEQ ID NO: 19 - DNA, 1923 bp, Artificial Sequence
  Synthetic
atgggccctggcttcgacttcgcccaggccatcatgaagaagaacaccgtgatcgccagaaccgagaagggcgagttcaccatgctgggcgtgtacgacagagtggccgtgatccctacccacgccagcgtgggcgaaaccatctacatcaacgacgtggaaacgaaggtgctggacgcctgcgccctgcgcgacctgaccgacaccaacctggagatcaccatcgtgaagctggacagaaaccagaagttcagagacatcagacacttcctgcctagatacgaggacgactacaacgacgccgtgctgagcgtgcacaccagcaagttccctaacatgtacatccctgtgggccaggtgaccaactacggcttcctgaacctgggcggcaccccaactcatagaatcctgatgtacaacttccctaccagagccggccagtgcggcggcgtggtgaccaccaccggcaaggtgatcggcatccacgtgggcggaaacggcgcccagggcttcgccgccatgctgctgcacagctacttcaccgatacccagggcgagatcgtgagcagcgagaagtccggcgtgtgcatcaacgcccctgccaagaccaagctgcagcctagcgtgttccaccaggtgttcgagggcagcaaggagcctgcagtactgaaccctaaggaccctagactgaagaccgacttcgaggaggccatcttcagcaagtatactggtaacaagatcatgctgatggacgagtacatggaagaggcggtggaccactacgtgggctgcctggagcctctggacatcagcgtggaccctatccctctggagagcgccatgtacggcatggacggcctggaggccctggacttgactactagcgccggcttcccttacctgctgcagggcaagaagaagcgagacatcttcaacagacacaccagagacaccagcgagatgaccaagatgctggagaagtacggtgtggatctgcctttcgtgaccttcgtgaaggacgagctgagaagcagagagaaggtggagaagggtaagagcagactgatcgaggccagcagcctgaacgacagcgtggccatgagagtggctttcggcaacctgtacgccaccttccacaacaaccctggcaccgctacgggtagcgccgtgggttgcgaccctgatatcttctggagcaagatccctatcttactggacggagagatattcgccttcgactacaccggctacgacgccagcctgagccctgtgtggttcgcctgcctgaagaaggtgttgatcaagctgggctacacccaccagaccagcttcatcgactacctgtgccactcggtgcatctgtacaaggacaagaagtatatcgtcaacggcggcatgcctagcggcagcagcggcacctctatcttcaataccatgatcaacaacatcatcatcagaaccctgctcattagagtctataagggcatcgacctggaccagttcaagatgatcgcctacggcgacgacgtaatcgccagctaccctcacaagatcgaccctggcctgctggccgaggccggcaagcagtacggactggtgatgacccctgccgacaagggaacttcgttcattgatactaattgggagaacgtcaccttccttaagagatacttcagagccgacgaccagtaccctttcctgatccaccctgtgatgcctatgaaggagatccacgagagcatccgctggaccaaggatccacgcaacacccaggaccacgtgagaagcctgtgctatctggcttggcacaacggcgaagaagcctataacgagttctgcagaaagatcagaagcgtgccagtgggcagggccctgaccctgcctgcctactcgtcgttgagaagaaagtggttggacagcttc

SEQ ID NO: 20 - protein, 641 aa, Artificial Sequence
  Synthetic
MGPGFDFAQAIMKKNTVIARTEKGEFTMLGVYDRVAVIPTHASVGETIYINDVETKVLDACALRDLTDTNLEITIVKLDRNQKFRDIRHFLPRYEDDYNDAVLSVHTSKFPNMYIPVGQVTNYGFLNLGGTPTHRILMYNFPTRAGQCGGVVTTTGKVIGIHVGGNGAQGFAAMLLHSYFTDTQGEIVSSEKSGVCINAPAKTKLQPSVFHQVFEGSKEPAVLNPKDPRLKTDFEEAIFSKYTGNKIMLMDEYMEEAVDHYVGCLEPLDISVDPIPLESAMYGMDGLEALDLTTSAGFPYLLQGKKKRDIFNRHTRDTSEMTKMLEKYGVDLPFVTFVKDELRSREKVEKGKSRLIEASSLNDSVAMRVAFGNLYATFHNNPGTATGSAVGCDPDIFWSKIPILLDGEIFAFDYTGYDASLSPVWFACLKKVLIKLGYTHQTSFIDYLCHSVHLYKDKKYIVNGGMPSGSSGTSIFNTMINNIIIRTLLIRVYKGIDLDQFKMIAYGDDVIASYPHKIDPGLLAEAGKQYGLVMTPADKGTSFIDTNWENVTFLKRYFRADDQYPFLIHPVMPMKEIHESIRWTKDPRNTQDHVRSLCYLAWHNGEEAYNEFCRKIRSVPVGRALTLPAYSSLRRKWLDSF

SEQ ID NO: 21 - DNA, 2762 bp, Artificial Sequence
  Synthetic
gggaaataagagagaaaagaagagtaagaagaaatataagaccccggcgccgccaccatgggaagtcaggtgtccactcaaagaagtggcagccacgagaacagcaacagcgccaccgagggcagcaccatcaactacacaacaatcaactactacaaagacagctacgccgctaccgcgggcaagcagagcctgaagcaggatccggacaagttcgctaatcctgtgaaggacatcttcaccgagatggccgcgccactgaagtctcctagcgccgaggcctgcggctacagcgacagggtggcccagctgaccatagggaacagcacaatcaccacccaggaggccgccaacatcatcgtgggttacggcgagtggcctagctactgtagcgatagcgacgccaccgccgtagataagccaaccagacctgacgtgagcgttaaccggttctacactctcgacactaagctttgggagaagtctagcaaaggttggtactggaagttccctgacgtgctgaccgaaaccggagtgttcggccagaacgcccagttccactacctgtatagaagcggcttttgcatccacgtacagtgcaacgcctctaagtttcaccagggcgcactgctggttgccgtcctgcctgaatacgttatcggcacggttgccggcggtacgggcaccgaggacacccacccgccctacaagcagacgcagcctggcgccgacggcttcgagcttcagcatccttacgtgttggacgccgggatccctatttctcagcttaccgtgtgccctcaccagtggatcaacctgagaaccaataactgcgcgacaatcatagtaccttacatcaacgccttgcctttcgacagcgccctgaatcactgcaacttcggcctgctcgtggtgcctatctcccctcttgattacgaccagggtgccacccctgtgatccccattaccatcaccctggcacctatgtgtagcgagttcgccggcctcaggcaggccgtgacccagggcttcccaaccgagctgaagccgggcaccaaccaattcctcactaccgacgacggcgtgagcgccccaatcctgcctaacttccaccctacaccgtgcatccacattcctggcgaggttagaaacctgctggagctttgtcaggtggagactattctggaagtgaataacgtgcctaccaacgccacaagtctgatggagagactgcgcttccctgtgagcgcgcaagccgggaagggcgagctgtgcgccgtgttccgagccgaccctggcagaaacggcccgtggcaaagcactctgctgggccagttgtgcggctactacacccagtggtccggcagcctcgaggtcaccttcatgttcaccggaagcttcatggccaccggcaagatgctgatcgcctacacccctcctggaggccctctgcctaaggacagagccactgccatgctggggacccacgtgatctgggacttcggactgcagtccagcgtgactctggtcatcccttggatctccaatacccactatagagctcacgccagagacggtgtgttcgactattacaccacaggcctggtttccatctggtaccagaccaattacgtggtgcccatcggcgcccctaacaccgcctatatcatcgccctggcagctgcccagaagaacttcaccatgaaactgtgcaaggacgcatcagacatcctgcagaccggcacgatccagggcgaccgagtggccgacgtgatcgagagttcaatcggcgacagcgtgagccgggccctgacccacgcactgcccgcccctacaggtcagaacacccaggtcagcagccaccgtctggatacaggcaaagtgcctgcgctgcaggccgcagagattggagccagctcaaacgcctccgacgagtcaatgatcgaaactaggtgcgtgctgaacagccatagcacagccgaaaccaccctggacagcttcttctctcgcgcaggactggtcggcgagatagacctgcctctggagggcaccaccaaccctaacggctacgcgaactgggacattgacatcaccggctacgcccaaatgaggagaaaggtggaactgttcacttacatgaggtttgacgccgaattcactttcgtggcgtgcaccccgaccggagaggtggtgcctcagctgctgcagtatatgtttgttccacctggcgcaccaaagcctgatagcagagagagcctggcctggcagaccgcaaccaatcctagtgtgttcgtgaagctgagcgatcctccagcccaggtgagcgtgcctttcatgagccctgctagcgcctaccagtggttttacgacgggtaccctaccttcggcgagcacaagcaggagaaggacttggagtacggcgcctgcccgaacaacatgatgggcaccttcagtgtgcggacggtcggaactagcaagagcaagtatcctctggtggtgaggatttacatgcgcatgaagcacgtccgtgcgtggatccctagacctatgcgtaaccagaactacctgttcaaggccaatccgaattacgccggcaattccatcaagcctacgggtgcgagccgcaccgcgattaccacactgtgataataggctggagcctcggtggcctagcttcttgccccttgggcctccccccagcccctcctccccttcctgcacccgtacccccgtggtctttgaataaagtctgagtgggcggc

SEQ ID NO: 22 - DNA, 2586 bp, Artificial Sequence
  Synthetic
atgggaagtcaggtgtccactcaaagaagtggcagccacgagaacagcaacagcgccaccgagggcagcaccatcaactacacaacaatcaactactacaaagacagctacgccgctaccgcgggcaagcagagcctgaagcaggatccggacaagttcgctaatcctgtgaaggacatcttcaccgagatggccgcgccactgaagtctcctagcgccgaggcctgcggctacagcgacagggtggcccagctgaccatagggaacagcacaatcaccacccaggaggccgccaacatcatcgtgggttacggcgagtggcctagctactgtagcgatagcgacgccaccgccgtagataagccaaccagacctgacgtgagcgttaaccggttctacactctcgacactaagctttgggagaagtctagcaaaggttggtactggaagttccctgacgtgctgaccgaaaccggagtgttcggccagaacgcccagttccactacctgtatagaagcggcttttgcatccacgtacagtgcaacgcctctaagtttcaccagggcgcactgctggttgccgtcctgcctgaatacgttatcggcacggttgccggcggtacgggcaccgaggacacccacccgccctacaagcagacgcagcctggcgccgacggcttcgagcttcagcatccttacgtgttggacgccgggatccctatttctcagcttaccgtgtgccctcaccagtggatcaacctgagaaccaataactgcgcgacaatcatagtaccttacatcaacgccttgcctttcgacagcgccctgaatcactgcaacttcggcctgctcgtggtgcctatctcccctcttgattacgaccagggtgccacccctgtgatccccattaccatcaccctggcacctatgtgtagcgagttcgccggcctcaggcaggccgtgacccagggcttcccaaccgagctgaagccgggcaccaaccaattcctcactaccgacgacggcgtgagcgccccaatcctgcctaacttccaccctacaccgtgcatccacattcctggcgaggttagaaacctgctggagctttgtcaggtggagactattctggaagtgaataacgtgcctaccaacgccacaagtctgatggagagactgcgcttccctgtgagcgcgcaagccgggaagggcgagctgtgcgccgtgttccgagccgaccctggcagaaacggcccgtggcaaagcactctgctgggccagttgtgcggctactacacccagtggtccggcagcctcgaggtcaccttcatgttcaccggaagcttcatggccaccggcaagatgctgatcgcctacacccctcctggaggccctctgcctaaggacagagccactgccatgctggggacccacgtgatctgggacttcggactgcagtccagcgtgactctggtcatcccttggatctccaatacccactatagagctcacgccagagacggtgtgttcgactattacaccacaggcctggtttccatctggtaccagaccaattacgtggtgcccatcggcgcccctaacaccgcctatatcatcgccctggcagctgcccagaagaacttcaccatgaaactgtgcaaggacgcatcagacatcctgcagaccggcacgatccagggcgaccgagtggccgacgtgatcgagagttcaatcggcgacagcgtgagccgggccctgacccacgcactgcccgcccctacaggtcagaacacccaggtcagcagccaccgtctggatacaggcaaagtgcctgcgctgcaggccgcagagattggagccagctcaaacgcctccgacgagtcaatgatcgaaactaggtgcgtgctgaacagccatagcacagccgaaaccaccctggacagcttcttctctcgcgcaggactggtcggcgagatagacctgcctctggagggcaccaccaaccctaacggctacgcgaactgggacattgacatcaccggctacgcccaaatgaggagaaaggtggaactgttcacttacatgaggtttgacgccgaattcactttcgtggcgtgcaccccgaccggagaggtggtgcctcagctgctgcagtatatgtttgttccacctggcgcaccaaagcctgatagcagagagagcctggcctggcagaccgcaaccaatcctagtgtgttcgtgaagctgagcgatcctccagcccaggtgagcgtgcctttcatgagccctgctagcgcctaccagtggttttacgacgggtaccctaccttcggcgagcacaagcaggagaaggacttggagtacggcgcctgcccgaacaacatgatgggcaccttcagtgtgcggacggtcggaactagcaagagcaagtatcctctggtggtgaggatttacatgcgcatgaagcacgtccgtgcgtggatccctagacctatgcgtaaccagaactacctgttcaaggccaatccgaattacgccggcaattccatcaagcctacgggtgcgagccgcaccgcgattaccacactg

SEQ ID NO: 23 - protein, 862 aa, Artificial Sequence
  Synthetic
MGSQVSTQRSGSHENSNSATEGSTINYTTINYYKDSYAATAGKQSLKQDPDKFANPVKDIFTEMAAPLKSPSAEACGYSDRVAQLTIGNSTITTQEAANIIVGYGEWPSYCSDSDATAVDKPTRPDVSVNRFYTLDTKLWEKSSKGWYWKFPDVLTETGVFGQNAQFHYLYRSGFCIHVQCNASKFHQGALLVAVLPEYVIGTVAGGTGTEDTHPPYKQTQPGADGFELQHPYVLDAGIPISQLTVCPHQWINLRTNNCATIIVPYINALPFDSALNHCNFGLLVVPISPLDYDQGATPVIPITITLAPMCSEFAGLRQAVTQGFPTELKPGTNQFLTTDDGVSAPILPNFHPTPCIHIPGEVRNLLELCQVETILEVNNVPTNATSLMERLRFPVSAQAGKGELCAVFRADPGRNGPWQSTLLGQLCGYYTQWSGSLEVTFMFTGSFMATGKMLIAYTPPGGPLPKDRATAMLGTHVIWDFGLQSSVTLVIPWISNTHYRAHARDGVFDYYTTGLVSIWYQTNYVVPIGAPNTAYIIALAAAQKNFTMKLCKDASDILQTGTIQGDRVADVIESSIGDSVSRALTHALPAPTGQNTQVSSHRLDTGKVPALQAAEIGASSNASDESMIETRCVLNSHSTAETTLDSFFSRAGLVGEIDLPLEGTTNPNGYANWDIDITGYAQMRRKVELFTYMRFDAEFTFVACTPTGEVVPQLLQYMFVPPGAPKPDSRESLAWQTATNPSVFVKLSDPPAQVSVPFMSPASAYQWFYDGYPTFGEHKQEKDLEYGACPNNMMGTFSVRTVGTSKSKYPLVVRIYMRMKHVRAWIPRPMRNQNYLFKANPNYAGNSIKPTGASRTAITTL

SEQ ID NO: 24 - DNA, 2114 bp, Artificial Sequence
  Synthetic
gggaaataagagagaaaagaagagtaagaagaaatataagaccccggcgccgccaccatgggccctagcctggacttcgccctgagcctgctgagaagaaacatcagacaggtgcagaccgaccagggccacttcaccatgctgggcgtgagagacagactggccgtgctgcctagacacagccagcctggcaagaccatctggatcgagcacaagctggtgaacgtgctggacgccgtggagctggtggacgagcagggcgtgaacctggagctgaccctgatcaccctggacaccaacgagaagttcagagacatcaccaagttcatccctgagaacatcagcaccgccagcgacgccaccctggtgatcaacaccgagcacatgcctagcatgttcgtgcctgtgggcgacgtggtgcagtacggcttcctgaacctgagcggcaagcctacccacagaaccatgatgtacaacttccctaccaaggctggacagtgtggcggcgtggtgaccagcgtgggcaaggtgatcggcatccacatcggcggcaacggcagacagggcttctgcgccggcctgaagagaagctacttcgcctctgaacagggtgagatccagtgggtgaagcctaacaaggagactggcagactgaacatcaacggccctaccagaaccaagctggagcctagcgtgttccacgacatcttcgagggcaataaggagccagccgtcctgcacagcaaggaccctagactggaggtggatttcgagcaggccctgttcagcaagtacgtcggcaacaccctgcacgagcctgacgagtacatcaaggaggccgccctgcactacgccaaccagctgaagcagctggagatcaatacgagccagatgagcatggaggaggcctgctacggcaccgagaacctcgaggccatcgacctgcacaccagcgccggctacccttacagcgccctgggcatcaagaagcgggacatcctggaccctaccaccagagacgtgagcaagatgaagttctacatggacaagtacggcctggacctgccatacagcacctacgtgaaggacgagctcaggagcatcgacaagattaagaagggcaagagtagactgatagaagccagcagcctgaacgacagcgtgtacctgagaatggccttcggccacctgtacgaggccttccacgctaatcctggcaccatcaccggcagcgccgtgggctgcaaccctgacaccttctggagcaagctgcctatcctgctgcctggcagcctgttcgccttcgactacagcggctacgacgcttctcttagccctgtgtggttcagagcactcgaactggtgctgagagagatcggctacagcgagggcgccgtgagcctcattgagggtatcaaccacacccaccacgtgtacagaaacaagacctactgcgtgttgggaggaatgccgagtggctgcagcggcaccagcatcttcaacagcatgattaataacatcatcatacgggccctactgatcaagaccttcaagggcattgacctggacgagttaaacatggtggcctacggcgacgacgtgctcgccagctacccgttccctatcgactgtcttgagctcgccaagaacggcaaggaatacggcttgaccatgacccctgccgacaagttcccttgcttcaaggaagtgaactggggcaacgccacgttcctgaagcgtggtttccttccagacgaacagttcccattcctgatccctcctaccatgcctatgcgcgaaatccacgagagcatccgctggaccaaggacgccagaaaccctcaggaccacgtgagaagcctgtgcctgctggcctggcacaacggcaagcaggagtacgagaagttcgtgagcacgataagaagcgtgccggtaggcagggcactggccatccctaactacgagaatctcaggaggaattggctggaactgttctgataataggctggagcctcggtggcctagcttcttgccccttgggcctccccccagcccctcctccccttcctgcacccgtacccccgtggtctttgaataaagtctgagtgggcggc

SEQ ID NO: 25 - DNA, 1938 bp, Artificial Sequence
  Synthetic
atgggccctagcctggacttcgccctgagcctgctgagaagaaacatcagacaggtgcagaccgaccagggccacttcaccatgctgggcgtgagagacagactggccgtgctgcctagacacagccagcctggcaagaccatctggatcgagcacaagctggtgaacgtgctggacgccgtggagctggtggacgagcagggcgtgaacctggagctgaccctgatcaccctggacaccaacgagaagttcagagacatcaccaagttcatccctgagaacatcagcaccgccagcgacgccaccctggtgatcaacaccgagcacatgcctagcatgttcgtgcctgtgggcgacgtggtgcagtacggcttcctgaacctgagcggcaagcctacccacagaaccatgatgtacaacttccctaccaaggctggacagtgtggcggcgtggtgaccagcgtgggcaaggtgatcggcatccacatcggcggcaacggcagacagggcttctgcgccggcctgaagagaagctacttcgcctctgaacagggtgagatccagtgggtgaagcctaacaaggagactggcagactgaacatcaacggccctaccagaaccaagctggagcctagcgtgttccacgacatcttcgagggcaataaggagccagccgtcctgcacagcaaggaccctagactggaggtggatttcgagcaggccctgttcagcaagtacgtcggcaacaccctgcacgagcctgacgagtacatcaaggaggccgccctgcactacgccaaccagctgaagcagctggagatcaatacgagccagatgagcatggaggaggcctgctacggcaccgagaacctcgaggccatcgacctgcacaccagcgccggctacccttacagcgccctgggcatcaagaagcgggacatcctggaccctaccaccagagacgtgagcaagatgaagttctacatggacaagtacggcctggacctgccatacagcacctacgtgaaggacgagctcaggagcatcgacaagattaagaagggcaagagtagactgatagaagccagcagcctgaacgacagcgtgtacctgagaatggccttcggccacctgtacgaggccttccacgctaatcctggcaccatcaccggcagcgccgtgggctgcaaccctgacaccttctggagcaagctgcctatcctgctgcctggcagcctgttcgccttcgactacagcggctacgacgcttctcttagccctgtgtggttcagagcactcgaactggtgctgagagagatcggctacagcgagggcgccgtgagcctcattgagggtatcaaccacacccaccacgtgtacagaaacaagacctactgcgtgttgggaggaatgccgagtggctgcagcggcaccagcatcttcaacagcatgattaataacatcatcatacgggccctactgatcaagaccttcaagggcattgacctggacgagttaaacatggtggcctacggcgacgacgtgctcgccagctacccgttccctatcgactgtcttgagctcgccaagaacggcaaggaatacggcttgaccatgacccctgccgacaagttcccttgcttcaaggaagtgaactggggcaacgccacgttcctgaagcgtggtttccttccagacgaacagttcccattcctgatccctcctaccatgcctatgcgcgaaatccacgagagcatccgctggaccaaggacgccagaaaccctcaggaccacgtgagaagcctgtgcctgctggcctggcacaacggcaagcaggagtacgagaagttcgtgagcacgataagaagcgtgccggtaggcagggcactggccatccctaactacgagaatctcaggaggaattggctggaactgttc

SEQ ID NO: 26 - protein, 646 aa, Artificial Sequence
  Synthetic
MGPSLDFALSLLRRNIRQVQTDQGHFTMLGVRDRLAVLPRHSQPGKTIWIEHKLVNVLDAVELVDEQGVNLELTLITLDTNEKFRDITKFIPENISTASDATLVINTEHMPSMFVPVGDVVQYGFLNLSGKPTHRTMMYNFPTKAGQCGGVVTSVGKVIGIHIGGNGRQGFCAGLKRSYFASEQGEIQWVKPNKETGRLNINGPTRTKLEPSVFHDIFEGNKEPAVLHSKDPRLEVDFEQALFSKYVGNTLHEPDEYIKEAALHYANQLKQLEINTSQMSMEEACYGTENLEAIDLHTSAGYPYSALGIKKRDILDPTTRDVSKMKFYMDKYGLDLPYSTYVKDELRSIDKIKKGKSRLIEASSLNDSVYLRMAFGHLYEAFHANPGTITGSAVGCNPDTFWSKLPILLPGSLFAFDYSGYDASLSPVWFRALELVLREIGYSEGAVSLIEGINHTHHVYRNKTYCVLGGMPSGCSGTSIFNSMINNIIIRALLIKTFKGIDLDELNMVAYGDDVLASYPFPIDCLELAKNGKEYGLTMTPADKFPCFKEVNWGNATFLKRGFLPDEQFPFLIPPTMPMREIHESIRWTKDARNPQDHVRSLCLLAWHNGKQEYEKFVSTIRSVPVGRALAIPNYENLRRNWLELF

SEQ ID NO: 27 - RNA, 9 nt, Artificial Sequence
  Synthetic
  misc_feature (3)..(3): R is A or G
ccrccaugg

SEQ ID NO: 28 - RNA, 11 nt, Artificial Sequence
  Synthetic
gggauccuacc

(19) United States
(12) Patent Application Publication
(10) Pub. No.: US 2024/0100145 A1
(43) Pub. Date: Mar. 28, 2024

(54) VLP ENTEROVIRAL VACCINES

(71) Applicant: Moderna TX, Inc., Cambridge, MA (US)

(72) Inventors: Brooke Bollman, Brookline, MA (US); Gaby Baldeon Vaca, Cambridge, MA (US); Angelo Cucuzza, Cambridge, MA (US); Yen-Ting Lai, Cambridge, MA (US)

(73) Assignee: Moderna TX, Inc., Cambridge, MA (US)

(21) Appl. No.: 18/280,362

(22) PCT Filed: Mar. 4, 2022
(86) PCT No.: PCT/US2022/019014

§ 371 Date: Sep. 5, 2023

Related U.S. Application Data
(60) Provisional application No. 63/157,543, filed on Mar. 5, 2021.

(51) Int. Cl. A61K 39/125; A61K 9/51; A61P 31/16; C07K 14/005; C12N 7/00; C12N 9/50

(52) U.S. Cl. CPC A61K 39/125; A61K 9/5123; A61P 31/16; C07K 14/005; C12N 7/00; C12N 9/506; A61K 2039/5258; C12Y 304/22028

(57) ABSTRACT

Aspects of the disclosure relate to compositions of messenger RNA vaccines and methods of administration thereof. Compositions provided herein include one or more RNA polynucleotides having an open reading frame encoding a picornavirus capsid polyprotein and a protease. Compositions provided herein include one or more RNA polynucleotides having an open reading frame encoding an active product that modulates the expression, structure or function of at least one other RNA or product thereof.

### RELATED APPLICATIONS

This application claims the benefit under 35 U.S.C. § 119(e) of U.S. Provisional Application Ser. No. 63/157,543, filed Mar. 5, 2021, entitled “VLP Enteroviral Vaccines,” which is incorporated by reference herein in its entirety.

### BACKGROUND

The Picornaviridae family of viruses, one of the largest virus families comprises the genus Enterovirus, contains many species of pathogens, such as species of Enterovirus and Rhinovirus, that are responsible for some of the most common infections in humans. It has been estimated that around 10-15 million symptomatic Enterovirus infections occur annually in the United States alone, excluding the very prevalent rhinovirus infections (van der Linden et al., 2015).

Vaccination is an effective way to provide prophylactic protection against infectious diseases, including, but not limited to, viral, bacterial, and/or parasitic diseases, such as influenza, HIV, hepatitis virus infection, cholera, malaria and tuberculosis, and many other diseases. However, developing vaccines targeting some Enteroviruses has proven difficult, at least in part because regions of conservation that would otherwise be appropriate for targeting are hidden on the interphase of the Enterovirus particle, while the difficult to target, highly variable regions are exposed on the surface of the Enterovirus particle.

Due to recent advances in recombinant DNA techniques, the use of virus-like particles (VLPs) have become of increasing interest for use in vaccine development. VLPs, which are formed by self-assembling viral structural proteins that mimic the morphology of a pathogen, have been shown to be both non-infective and highly immunogenic. However, previous attempts to generate an Enterovirus VLP have been met with limited success, due to difficulties achieving proper expression and/or folding of the structural proteins required to form a VLP that mimics the Enterovirus pathogen.

The present disclosure describes how mRNA can be used to encode and deliver both a catalytic enzymatic protein and a polyprotein encoding multiple substrates of the enzyme and subsequently allows the enzyme to facilitate building of a higher order structure in the form of a VLP to serve as an immunogen in vivo. It is therefore of great interest to develop Enterovirus VLPs from mRNAs for use as Enterovirus vaccines as a new approach to combatting infectious disease and infectious agents.

### SUMMARY

In view of the lack of vaccines effective against infections caused by Enteroviruses, with the exception of the Polio vaccine, there is a significant need for a vaccine that would be safe and effective in all patient populations to prevent and/or to treat other types of Enterovirus infections. Described herein are compositions and methods of nucleic acid vaccines. In particular, described herein are compositions and methods of nucleic acid vaccines (e.g., mRNA vaccines).

Aspects of the disclosure relate to compositions comprising: (i) a messenger ribonucleic acid (mRNA) comprising an open reading frame (ORF) encoding a picornavirus capsid polyprotein, wherein the capsid polyprotein comprises a viral P1 precursor polyprotein; and (ii) a lipid nanoparticle (LNP).

Further aspects of the disclosure relate to compositions comprising: (i) a messenger ribonucleic acid (mRNA) comprising an open reading frame (ORF) encoding a protease, wherein the protease is a picornavirus 3C protease; and (ii) a lipid nanoparticle (LNP).

Further aspects of the disclosure relate to immunogenic compositions comprising: (i) a messenger ribonucleic acid (mRNA) comprising an open reading frame (ORF) encoding a picornavirus 3C protease; (ii) an mRNA comprising an ORF encoding a picornavirus capsid polyprotein, wherein the capsid polyprotein comprises a viral P1 precursor polyprotein; and (iii) a lipid nanoparticle (LNP).

In some embodiments, the precursor polyprotein comprises two or more capsid proteins and has a cleavage site specific for a viral protease between the two or more capsid proteins. In some embodiments, the two or more capsid proteins comprise two or more of viral protein 0 (VP0), viral protein 1 (VP1), and viral protein 3 (VP3). In some embodiments, VP0 further comprises viral protein 2 (VP2) and viral protein 4 (VP4), and wherein VP2 and VP4 comprise a cleavage site for capsid maturation.

In some embodiments, the protease is Picornavirus 3C (3CD). In some embodiments, the 3CD is specific to a species in the genus Enterovirus in the picornavirus family. In some embodiments, the 3CD is specific to a species of human rhinovirus (HRV) A, B or C. In some embodiments, the HRV species is HRV-A16. In some embodiments, the HRV species is HRV-B14. In some embodiments, the 3CD is specific to an Enterovirus species. In some embodiments, the Enterovirus genotype is EV-D68. In some embodiments, the Enterovirus genotype is EV-A71.

In some embodiments, the capsid proteins form a protomer. In some embodiments, the protomers form a pentamer. In some embodiments, the pentamers form a virus-like particle (VLP).

In some embodiments, mRNA comprising the ORF encoding the viral P1 precursor polyprotein and the mRNA comprising the ORF encoding the picornavirus 3C protease (P1:3CD) are present in one of the following ratios: 20:1, 10:1, 7:1, 5:1, 4:1, 3:1, 2:1, 1:1. In some embodiments, the ratio of mRNA comprising the ORF encoding the viral P1 precursor protein and the mRNA comprising the ORF encoding the picornavirus 3C protease is 10:1. In some embodiments, the ratio of mRNA comprising the ORF encoding the viral P1 precursor polyprotein and the mRNA comprising the ORF encoding 3C protease as either 3C or 3CD is 2:1. In some embodiments, the protease comprises an amino acid sequence having at least 90% identity to the amino acid sequence of SEQ ID NO: 8 or SEQ ID NO: 14. In some embodiments, the protease comprises the amino acid sequence of SEQ ID NO: 8 or SEQ ID NO: 14. In some embodiments, the protease comprises an amino acid sequence having at least 90/6 identity to the amino acid sequence of SEQ ID NO: 20 or SEQ ID NO: 26. In some embodiments, the protease comprises the amino acid sequence of SEQ ID NO: 20 or SEQ ID NO: 26.

In some embodiments, the capsid polyprotein comprises an amino acid sequence having at least 90% identity to the amino acid sequence of any one of SEQ ID NOs: 5, 11, 17, or 23. In some embodiments, capsid polyprotein comprises the amino acid sequence of any one of SEQ ID NOs: 5, 11, 17, or 23. In some embodiments, the VLP comprises Neutralizing Immunogenic (NIm) sites.

In some embodiments, the LNP comprises an ionizable amino lipid, a PEG-modified lipid, a structural lipid and a phospholipid.

In some embodiments, the mRNA comprising the ORF encoding the viral P1 precursor polyprotein and the mRNA comprising the ORF encoding the picornavirus 3C protease are co-formulated in at least one LNP. In some embodiments, the mRNA comprising the ORF encoding viral P1 precursor polyprotein and the mRNA comprising the ORF encoding the picornavirus 3C protease are each formulated in separate LNPs. In some embodiments, the LNP formulated with mRNA comprising the ORF encoding the viral P1 precursor polyprotein and the mRNA comprising the ORF encoding the picornavirus 3C protease are present in one of the following ratios: 20:1, 10:1, 7:1, 5:1, 4:1, 3:1, 2:1, 1:1. In some embodiments, the LNP comprises an ionizable amino lipid, a sterol, neutral lipid, and a PEG-modified lipid. In some embodiments, the lipid nanoparticle comprises 40-55 mol % ionizable amino lipid, 30-45 mol % sterol, 5-15 mol % neutral lipid, and 1-5 mol % PEG modified lipid. In some embodiments, the lipid nanoparticle comprises 40-50 mol % ionizable amino lipid, 35-45 mol % sterol, 10-15 mol % neutral lipid, and 2-4 mol % PEG-modified lipid. In some embodiments, the lipid nanoparticle comprises 45 mol %, 46 mol %, 47 mol %, 48 mol %, 49 mol %, or 50 mol % ionizable amino lipid. In some embodiments, the ionizable amino lipid has the structure of Compound 2:

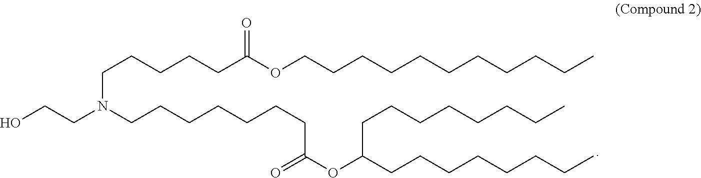 [SMILES: CCCCCCCCCCCOC(=O)CCCCCN(CCO)CCCCCCCC(=O)OC(CCCCCCCC)CCCCCCCC]

In some embodiments, the sterol is cholesterol or a variant thereof. In some embodiments, the neutral lipid is 1,2 distearoyl-sn-glycero-3-phosphocholine (DSPC).

Further aspects of the disclosure relate to methods comprising administering to a subject an immunogenic composition comprising: (i) a messenger ribonucleic acid (mRNA) comprising an open reading frame encoding a picornavirus protease; and (ii) a messenger ribonucleic acid (mRNA) comprising an open reading frame encoding a capsid polyprotein comprising a precursor protein, wherein the precursor protein comprises two or more capsid proteins and has a cleavage site specific for the protease between the two or more capsid proteins, in an amount effective to induce in the subject an immune response against a viral infection from a member of the Enterovirus genus.

In some embodiments, the immune response includes a binding antibody titer to a human rhinovirus species of the Enterovirus genus. In some embodiments, the immune response includes a neutralizing antibody titer to a human rhinovirus species of the Enterovirus genus. In some embodiments, the immune response includes a T cell response to a human rhinovirus species of the Enterovirus genus. In some embodiments, the human rhinovirus species of virus is selected from the group consisting of genotypes A2, A16, B14, and C15. In some embodiments, the human rhinovirus genotype is human rhinovirus 16 (HRV16).

In some embodiments, the immune response includes a binding antibody titer to a human enterovirus species of the Enterovirus genus. In some embodiments, the immune response includes a neutralizing antibody titer to a human enterovirus species of the Enterovirus genus. In some embodiments, the immune response includes a T cell response to a human enterovirus species of the Enterovirus genus. In some embodiments, the human enterovirus species of virus is selected from the group consisting of RV-A, RV-B, RV-C, EV-A and EV-D. In some embodiments, the human enterovirus species of virus is selected from the group consisting of genotypes RV-A16, RV-B14, RV-C15, EV-A71 and EV-D68. In some embodiments, the human enterovirus species of virus is Enterovirus D68 (EV-D68). In some embodiments, the human enterovirus species of virus is Enterovirus A71 (EV-A71).

In some embodiments, the mRNA of (i) are formulated in a composition comprising at least one lipid nanoparticle. In some embodiments, the mRNA of (ii) are formulated in a composition comprising at least one lipid nanoparticle. In some embodiments, the mRNA of (i) are administered to the subject at the same time as the mRNA of (ii). In some embodiments, the mRNA of (i) and (ii) are formulated in a composition comprising at least one lipid nanoparticle. In some embodiments, the mRNA of (i) and (ii) are formulated in a composition comprising two lipid nanoparticles.

Further aspects of the disclosure relate to compositions comprising: (i) a first messenger ribonucleic acid (mRNA) comprising an open reading frame (ORF) encoding a first product; (ii) a second mRNA comprising an ORF encoding a second product; and (iii) a lipid nanoparticle (LNP), wherein the first product is an active protein and wherein the active protein can modulate the expression, structure, or activity of the second mRNA and/or the second product. In some embodiments, the composition is an immunogenic composition. In some embodiments, the first product is a catalytic protein. In some embodiments, the first product is an enzymatic protein. In some embodiments, the first product is a binding protein. In some embodiments, the first product is a polyprotein. In some embodiments, the second product is a substrate. In some embodiments, the first product is a polyprotein.

Each of the limitations of the disclosure can encompass various embodiments of the disclosure. It is, therefore, anticipated that each of the limitations of the disclosure involving any one element or combinations of elements can be included in each aspect of the disclosure. This disclosure is not limited in its application to the details of construction and the arrangement of components set forth in the following description or illustrated in the drawings. The disclosure is capable of other embodiments and of being practiced or of being carried out in various ways.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1B depicts a schematic of the enterovirus genome and the resulting icosahedral virion. FIG. 1A is a schematic depicting the relative scale of capsid proteins (VP1, VP2, VP3, and VP4) and non-structural proteins (2A, 2B, 2C, 3A, 3B, 3C, and 3D). Protease cleavage sites on the polyproteins (P1, P2, and P3) are indicated by inverted triangles. FIG. 1B depicts the resulting enterovirus icosahedral virion following polyprotein processing and assembly. The capsid proteins VP1, VP2, and VP3 are shown on the outside of the icosahedral virion.

FIGS. 2A-2D depicts an mRNA vaccine composed of HRV-A16 P1+3CD mRNAs demonstrating proper P1 processing in vitro and generating neutralizing antibody titers in mice. FIG. 2A is a western blot depicting that cell lysates from 293T cells transfected with 2 μg HRV-A16 P1 mRNA and increasing amounts of HRV-A16 3CD mRNA, to give the indicated P1:3CD molar ratios. The results show P1 is proteolytically processed to individual VP proteins. Cleavage is demonstrated by disappearance of the high molecular weight P1 band and appearance of a lower molecular weight band corresponding to VP0. Wild-type HRV-A16 virion lysate is included for molecular weight comparison, with arrows indicating P1 cleavage products. Cell lysates blotted for GAPDH are shown as a loading control. FIG. 2B depicts Virus-like particles (VLPs) produced by co-expression of P1 and 3CD. Expi293 cells were transfected with plasmids encoding P1 and 3CD. VLPs were purified from the supernatant via PEG precipitation followed by anion exchange chromatography. VLPs were eluted and imaged using negative stain transmission electron microscopy. A 200 nm scale bar is included for reference. FIG. 2C a graph depicting vaccination with HRV-A16 P1:3CD mRNA generates detectable neutralizing antibody titers in mice, at levels comparable to those elicited by injection of replicative virus. Mice were vaccinated with 10 μg P1 mRNA and varying amounts of 3CD mRNA to make up the molar ratios of 10:1 and 2:1 P1:3CD. Mice were vaccinated on days 1 and 22, and serum was collected on day 21 (post-dose 1, gray) and day 35 (post-dose 2, black) and tested in the HRV-A16 neutralization assay. Serum from cotton rats that had received injections of replicative HRV-A16 on Days 1, 22 was tested in parallel (Cotton Rat HRV-A16 A/S). Horizontal cross bars represent geometric mean titer (GMT) and vertical bars represent standard deviation. Dotted line represents assay limit of detection (LOD)=25. Animals <LOD were assigned a titer of 12.5. FIG. 2D is a graph depicting Vaccination with EV-D68 P1:3CD mRNA at varying ratios generates neutralizing antibody titers against two heterologous viral isolates. Mice were vaccinated with 10 μg P1 mRNA and varying amounts of 3CD mRNA to makeup the final molar ratios of 10:1, 5:1, 2:1 and 1:1 P1 mRNA: 3CD mRNA. Mice were vaccinated on days 1 and 22, and the serum collected on day 35 was pooled for each group. Pooled group serum was tested for neutralization of EV-D68 Clade B1 isolate US/MO/14-18947 and Clade A2 isolate US/KY/14-18953. Dotted line represents assay limit of quantitation (LOQ=100).

FIGS. 3A-3D are graphs depicting the neutralizing antibody response to vaccination with P1+3CD mRNAs at a 2:1 molar ratio for four distinct Enterovirus species. FIG. 3A is a graph depicting Vaccination with HRV-A16 P1+3CD mRNA leads to dose-dependent production of neutralizing antibodies. X axis indicates μg amount of P1 and 3CD mRNA given for each dose group. Bars represent GMT for the group of 8 mice, with individual animals represented by dots. Error bars represent standard deviation. Commercial HRV-A16 antiserum from hyperimmune guinea pigs is included as a benchmark control (GP A/S). Dotted line at Y axis represents assay LOD=25. FIG. 3B is a graph depicting Vaccination with HRV-B14 P1+3CD mRNA at doses of 5 μg, 1 μg, and 0.2 μg P1 mRNA leads to similar levels of neutralizing antibodies and exceeds the neutralizing antibody titer of commercial hyperimmune antiserum. X axis indicates μg amount of P1 and 3CD mRNA given for each dose group. Bars represent GMT for the group of 8 mice, with individual animals represented by dots. Error bars represent standard deviation. Commercial HRV-B14 antiserum from hyperimmune guinea pigs is included as a benchmark control (GP A/S). Dotted line at Y axis represents assay LOD=25. FIG. 3C is a graph depicting vaccination with EV-D68 P1+3CD mRNA at doses of 5, 1 and 0.2 μg P1 mRNA raises a strong neutralizing antibody response even at low doses. Serum was tested for neutralization of EV-D68 Clade B1 isolate US/MO/14-18947 and Clade A2 isolate US/KY/14-18953. X axis indicates μg amount of P1 and 3CD mRNA given for each group. Bars represent GMT for the group of 8 mice, with individual animals represented by dots. Error bars indicate std dev. Dotted lines at Y axis represent assay limit of detection (LOD)=25. Animals with titers <LOD were assigned a value of 12.5. Bar shades correspond to legend and represent virus isolate tested. FIG. 3D is a graph depicting vaccination with EV-A71 P1+3CD mRNA leads to dose-dependent production of antibodies that can neutralize a clinical isolate recovered from a patient suffering from acute flaccid myelitis. X axis indicates μg amount of P1 and 3CD mRNA given for each group. Bars represent GMT for each dose group. Error bars indicate std dev. Dotted line at Y axis represents assay LOD=20. Animals with titers <LOD were assigned a titer value of 10.

FIGS. 4A-4B depict that a combination of P1+3CD from two different species demonstrates P1 processing in vitro and is immunogenic in vivo. FIG. 4A is a western blot depicting cell lysates from 293T cells transfected with 2 μg of P1 mRNA 0.75 μg of 3CD mRNA were blotted for P1 processing. Lane labels at bottom of blot indicate HRV species of transfected P1 and 3CD mRNA. HRV-B14 P1 is cleaved when co-transfected with 3CD mRNA from either HRV-B14 or HRV-A16. Cleavage is indicated by the disappearance of the high molecular weight band corresponding to P1 and appearance of a lower molecular weight band corresponding to VP1. Cell lysates blotted for GAPDH are shown as a loading control. FIG. 4B is a graph depicting vaccination with a combination of HRV-B14 P1+HRV-A16 3CD mRNA produces anti-HRV-B14 neutralizing antibodies. Mice were vaccinated on Day 1 and Day 22 with 2.5 μg of the indicated P1 mRNA+0.94 μg of HRV-A16 3CD. Serum collected on Day 35 was tested for neutralization of either HRV-A16 or HRV-B14 virus. Commercial hyperimmune antiserum for each virus was included as a benchmark control. Solid bars represent GMT for the group of 8 mice, with individual animals represented by dots. Error bars represent standard deviation. Hashed bars represent the neutralizing titer for the hyperimmune antisera. Dotted line at Y axis represents assay LOD=25.

FIGS. 5A-5D are graphs depicting vaccination with a combination of P1/3CD mRNA provides dose-dependent protection from HRV16 challenge in cotton rats. FIG. 5A is a graph depicting animals vaccinated with a prime-boost regimen of P1/3CD exhibit dose-dependent production of nAb. Four out of five animals in each dose group that received the higher doses of P1/3CD (25, 5 and 1 μg), and 5/5 animals vaccinated with live HRV16, showed detectable HRV16 nAb at time of challenge (2 weeks post-boost). Animals vaccinated with 0.2 μg P1/3CD, or animals receiving PBS as the negative control, had no detectable nAb titers. FIG. 5B is a graph depicting vaccination with P1/3CD leads to a dose-dependent protection of lung tissue. Viral load in lung tissue was measured 8 h post-HRV16 challenge. Four out of 5 animals that received 25 μg P1/3CD mRNA and 3/5 animals that received 5 μg showed complete protection, and all animals vaccinated with 1 μg showed reduced viral load as compared to PBS-vaccinated control. No protection of lung tissue was found in animals receiving the lowest (0.2 μg) dose. Animals vaccinated with a prime-boost regimen of live HRV16 were included as a positive control and comparator for protection. FIG. 5C is a graph depicting vaccination with P1/3CD leads to complete protection of nose tissue at the highest dose and some protection at lower doses. Five out of 5 animals that received 25 μg P1/3CD had no detectable viral load in nose tissue at 8 h post-HRV16 challenge. Animals vaccinated with 5 μg or 1 μg showed complete protection in 2/5 animals. No protection of the nose was seen in animals receiving 0.2 μg. FIG. 5D is a graph depicting vaccination with P1/3CD leads to a reduction in lung viral load as measured by qPCR for HRV16 (−)vRNA. A strong reduction in lung viral titers was seen in 5/5 animals that received 25 μg P1/3CD, and in some animals that received 5 μg or 1 μg. No reduction was seen in animals vaccinated with 0.2 μg. For each panel, bars represent GMT for the group of 5 cotton rats, with individual animals represented by dots. Error bars represent geometric standard deviation. Dotted line at Y axis represents assay LOD=200.

FIG. 6 is a table depicting individual cotton rat endpoint assay results for HRV-A16 vaccination+challenge study.

### DETAILED DESCRIPTION

It is of great interest in the fields of therapeutics, diagnostics, reagents and for biological assays to be able design, synthesize and deliver a nucleic acid, e.g., a ribonucleic acid (RNA) inside a cell, whether in vitro, in vivo, in situ or ex vivo, such as to effect physiologic outcomes which are beneficial to the cell, tissue or organ and ultimately to an organism. One beneficial outcome is to cause intracellular translation of the nucleic acid and production of at least one encoded peptide or polypeptide of interest.

Virus-like particles (VLPs) are spherical particles that closely resemble live viruses in structural characteristics and antigenicity. However, VLPs are distinguished from live viruses in that VLPs do not comprise any viral genetic material and are therefore non-infective. Due to their antigenic, yet non-infective nature, there is an increased interest in exploring the application of VLPs in vaccinations.

Currently, the majority of VLPs are generated using recombinant or cloning strategies. A VLP may be a self-assembled particle. Non-limiting examples of self-assembled VLPs and methods of making the self-assembled VLPs are described in International Patent Publication No. WO2013122262, the contents of which are herein incorporated by reference in its entirety.

VLPs are formed from the assembly of structural viral proteins (e.g., envelope and/or capsid proteins). The size and morphology of a VLP depends, at least in part, on the particular structural viral proteins that are incorporated into the particle upon assembly. A VLP assembled from the structural viral proteins of an enveloped virus may comprise, for example, one or more envelope proteins and one or more capsid proteins. A VLP assembled from the structural viral proteins of a non-enveloped virus may comprise, for example, one or more capsid proteins.

Multiple capsid proteins may be assembled by co-expression of the capsid proteins from bicistronic or multicistronic vectors in the same cell. Although attempts have been made to produce VLPs that mimic viruses from the Enterovirus genus, including expressing viral structural proteins on different vectors within the same cell and/or designing fusion proteins of viral structural proteins with chaperone proteins, the resulting VLPs do not form properly such that they fail to mimic the morphology of an Enterovirus VLP.

Quite surprisingly, the inventors have discovered, according to aspects of the invention, that multiple RNAs can be delivered such that one of the RNAs produces a protein which acts on the other RNA or protein produced by the RNA thus achieving a complex physiological process within the cell. For instance, in some cases a complex structure such as a VLP may be assembled properly from one or more capsid precursor polyproteins which are expressed and subsequently processed in a cell from messenger ribonucleic acid (mRNA) that is delivered in lipid nanoparticles (LNPs). For instance, the inventors identified that compositions comprising a mRNA comprising an open reading frame (ORF) encoding a picornavirus capsid polyprotein and a mRNA comprising an ORF encoding a picornavirus 3C protease are sufficient for the formation of a virus-like particle. In some aspects, the compositions are formulated into one or more lipid nanoparticles.

The inventors have also discovered, according to aspects of the invention methods comprising administering an immunogenic composition comprising a mRNA comprising an ORF encoding a picornavirus capsid polyprotein and optionally a mRNA comprising an ORF encoding a picornavirus 3C protease in an amount effective to induce in the subject an immune response against a viral infection from a member of the Enterovirus genus.

Further aspects of the disclosure relate to compositions in which two or more mRNAs are delivered to a subject, wherein the mRNA encode at least a first and second product that are able to interact and achieve an end result in the body. In the example above, one product is a precursor protein or polyprotein (substrate) and the other is an enzyme. In other words the first product is an active protein and that active protein can modulate the expression, structure, or activity of the second mRNA and/or the second product. The two products may be a substrate enzyme pair. Alternatively the first product may be a binding protein that influences the second mRNA translation process or the function of the second product.

Described herein are compositions comprising one or more polynucleotides encoding a picornavirus capsid polyprotein. As such the present invention is directed, in part, to polynucleotides, specifically messenger ribonucleic acid (mRNA) comprising an open reading frame encoding one or more picornavirus capsid polyprotein and/or components thereof. In some embodiments, the picornavirus capsid polyprotein comprises two or more capsid proteins and has a cleavage site specific for a viral protease between the two or more capsid proteins.

Further described herein are compositions comprising one or more polynucleotides encoding a picornavirus 3C protease. As such the present invention is directed, in part, to polynucleotides, specifically mRNA comprising an open reading frame encoding a picornavirus 3C protease.

Further described herein are compositions comprising one or more polynucleotides encoding a picornavirus capsid polyprotein and one or more polynucleotides encoding a picornavirus 3C protease. As such the present invention is directed, in part, to polynucleotides, specifically a mRNA comprising an open reading frame encoding a picornavirus capsid polyprotein and a mRNA comprising an open reading frame encoding a picornavirus 3C protease.

In some embodiments of the present invention, a precursor polyprotein is a capsid polyprotein. In some embodiments, the capsid polyprotein is a picornavirus capsid polyprotein. In some embodiments, the picornavirus capsid polyprotein is a viral P1 precursor polyprotein.

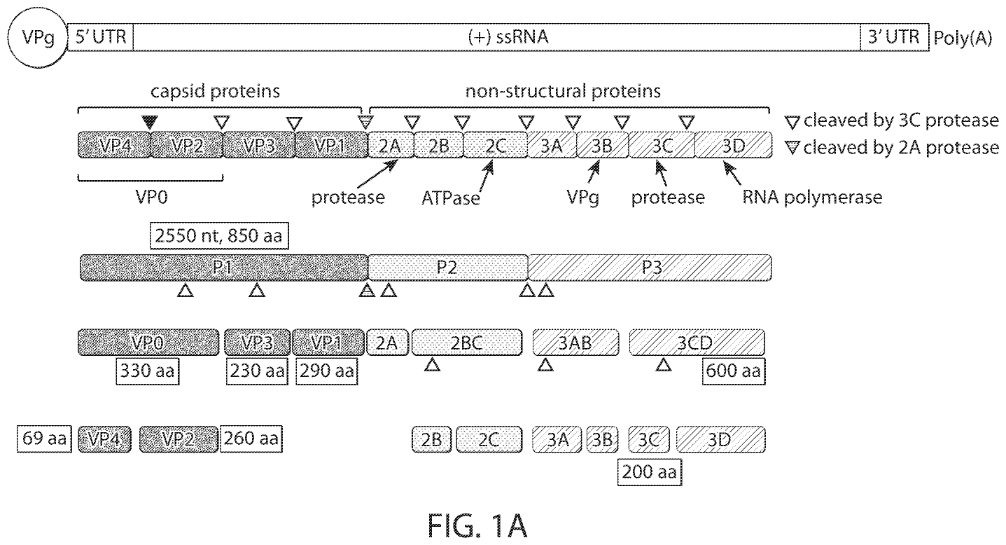

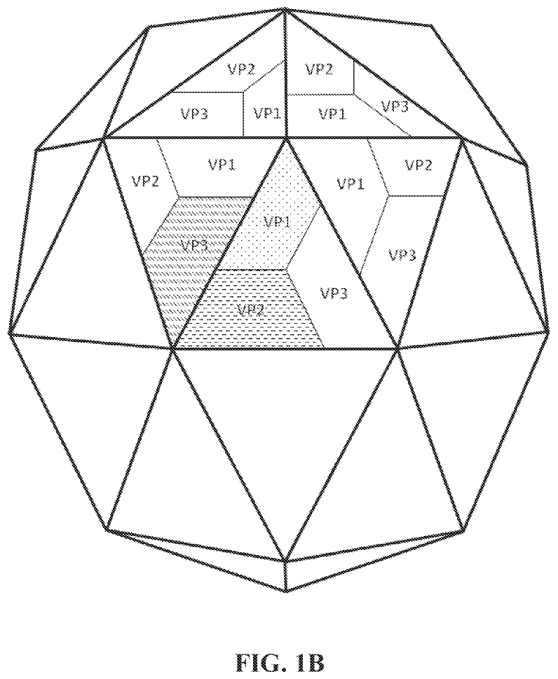

A picornavirus capsid polyprotein may be encoded by a single ribonucleic acid (RNA) molecule, which can be a bicistronic molecule encoding two separate polypeptide chains or can be a polycistronic molecule encoding three separate polypeptide chains (FIG. 1A). Such an RNA molecule may contain a signal sequence between the two or three coding sequences such that two or three separate polypeptides would be produced in the translation process. Alternatively, the RNA molecule may include a sequence coding for a cleavage site (e.g., a protease cleavage site) in between the capsid proteins such that it produces a single capsid polyprotein, which can be processed via cleavage at the cleavage site to produce the two or more separate capsid proteins. Alternatively, the capsid proteins may be encoded by two or three separate RNA molecules.

In some embodiments, a capsid polyprotein comprises two or more capsid proteins. In some embodiments, the two or more capsid proteins comprise two or more of a viral protein 0 (VP0), viral protein 1 (VP1), and viral protein 3 (VP3). In some embodiments, the viral protein 0 (VP0) further comprises viral protein 2 (VP2) and viral protein 4 (VP4).

A capsid polyprotein comprising two or more capsid proteins may further comprise one or more cleavage sites specific for a viral protease. For example, in some embodiments of the present invention, a precursor polyprotein comprises two or more capsid proteins and has a cleavage site specific for a viral protease between the two or more capsid proteins. In some embodiments, there is a cleavage site specific for a viral protease between one or more of VP0, VP1, and VP3. A capsid polyprotein may further comprise a cleavage site for capsid maturation. In some embodiments, a capsid polyprotein that comprises VP0, further comprises VP2 and VP4, wherein VP2 and VP4 comprise a cleavage site for capsid maturation (FIG. 1A).

In some embodiments, the mRNA encoding a picornavirus capsid polyprotein is specific to a human rhinovirus (HRV) species. In some embodiments, the HRV species is HRV-A16. In some embodiments, the mRNA encoding a picornavirus capsid polyprotein comprises a nucleotide sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 3. In some embodiments, the nucleotide sequence of the mRNA encoding a picornavirus capsid polyprotein comprises SEQ ID NO: 3. In some embodiments, the HRV species is HRV-B14. In some embodiments, the mRNA encoding a picornavirus capsid polyprotein comprises a nucleotide sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 10. In some embodiments, the nucleotide sequence of the mRNA encoding a picornavirus capsid polyprotein comprises SEQ ID NO: 10.

In some embodiments, the picornavirus capsid polyprotein is specific to a human rhinovirus (HRV) species. In some embodiments, the HRV species is HRV-A16. In some embodiments, the picornavirus capsid polyprotein comprises an amino acid sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 5. In some embodiments, the amino acid sequence of the picornavirus capsid polyprotein comprises SEQ ID NO: 5. In some embodiments, the HRV species is HRV-B14. In some embodiments, the picornavirus capsid polyprotein comprises an amino acid sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 11. In some embodiments, the amino acid sequence of the picornavirus capsid polyprotein comprises SEQ ID NO: 11.

In some embodiments, the mRNA encoding a picornavirus capsid polyprotein is specific to an Enterovirus species. In some embodiments, the Enterovirus species is EV-D68. In some embodiments, the mRNA encoding a picornavirus capsid polyprotein comprises a nucleotide sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 16. In some embodiments, the nucleotide sequence of the mRNA encoding a picornavirus capsid polyprotein comprises SEQ ID NO: 16. In some embodiments, the Enterovirus species is EV-A71. In some embodiments, the mRNA encoding a picornavirus capsid polyprotein comprises a nucleotide sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 22. In some embodiments, the nucleotide sequence of the mRNA encoding a picornavirus capsid polyprotein comprises SEQ ID NO: 22.

In some embodiments, the picornavirus capsid polyprotein is specific to an Enterovirus species. In some embodiments, the Enterovirus species is EV-D68. In some embodiments, the picornavirus capsid polyprotein comprises an amino acid sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 17. In some embodiments, the amino acid sequence of the picornavirus capsid polyprotein comprises SEQ ID NO: 17. In some embodiments, the Enterovirus species is EV-A71. In some embodiments, the picornavirus capsid polyprotein comprises an amino acid sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 23. In some embodiments, the amino acid sequence of the picornavirus capsid polyprotein comprises SEQ ID NO: 23.

Prior to assembly of a VLP, a capsid polyprotein may be processed or cleaved by a non-structural viral protein (e.g., a viral protease). As used herein, a viral protease refers to a protease that may recognize a cleavage site specific for a viral protease between one or more capsid proteins within a capsid polyprotein. In some embodiments, the viral protease is a picornavirus 3C (3CD) protease. The viral protease 3CD is part of non-structural polyprotein P3 (FIG. 1A). The viral protease 3CD may be cleaved into 3C and 3D. A picornavirus 3C protease can be supplied by 3C or 3CD. In some embodiments, the picornavirus 3C protease is supplied by 3C. In some embodiments, the 3C protease is supplied by 3CD. In some embodiments, the 3CD protease is specific to a species in the genus Enterovirus.

In some embodiments, the mRNA encoding a picornavirus 3C protease (3CD) is specific to a human rhinovirus (HRV) species. In some embodiments, the HRV species is HRV-A16. In some embodiments, the mRNA encoding a 3CD comprises a nucleotide sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 7. In some embodiments, the nucleotide sequence of the mRNA encoding the 3CD comprises SEQ ID NO: 7. In some embodiments, the HRV species is HRV-B14. In some embodiments, the mRNA encoding a 3CD comprises a nucleotide sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 13. In some embodiments, the nucleotide sequence of the mRNA encoding the 3CD comprises SEQ ID NO: 13.

In some embodiments, the 3CD protease is specific to a human rhinovirus (HRV) species. In some embodiments, the HRV species is HRV-A16. In some embodiments, the 3CD protease comprises an amino acid sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 8. In some embodiments, the amino acid sequence of the 3CD protease comprises SEQ ID NO: 8. In some embodiments, the HRV species is HRV-B14. In some embodiments, the 3CD protease comprises an amino acid sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 14. In some embodiments, the amino acid sequence of the 3CD protease comprises SEQ ID NO: 14.

In some embodiments, the mRNA encoding a picornavirus 3C protease (3CD) is specific to an Enterovirus species. In some embodiments, the Enterovirus species is EV-D68. In some embodiments, the mRNA encoding a 3CD comprises a nucleotide sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 19. In some embodiments, the nucleotide sequence of the mRNA encoding the 3CD comprises SEQ ID NO: 19. In some embodiments, the Enterovirus species is EV-A71. In some embodiments, the mRNA encoding a 3CD comprises a nucleotide sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 25. In some embodiments, the nucleotide sequence of the mRNA encoding the 3CD comprises SEQ ID NO: 25.

In some embodiments, the 3CD protease is specific to an Enterovirus species. In some embodiments, the Enterovirus genotype is EV-D68. In some embodiments, the 3CD protease comprises an amino acid sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 20. In some embodiments, the amino acid sequence of the 3CD protease comprises SEQ ID NO: 20. In some embodiments, the Enterovirus genotype is EV-A71. In some embodiments, the 3CD protease comprises an amino acid sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 26. In some embodiments, the amino acid sequence of the 3CD protease comprises SEQ ID NO: 26.

According to the present invention, the polynucleotides are described in compositions wherein the polynucleotides encoding one or more picornavirus capsid polyprotein and/or picornavirus 3C protease thereof are present in a composition in a specific ratio. As used herein, a “ratio” describes the proportion of a picornavirus 3C protease to a picornavirus capsid polyprotein that is capable of producing a VLP. In some embodiments, a ratio refers to a molar ratio. In some embodiments, a ratio refers to a mass ratio.

In some embodiments, the ratio of an mRNA comprising an open reading frame encoding a picornavirus 3C protease to a mRNA comprising an open reading frame encoding a picornavirus capsid polyprotein is at least 20:1, at least 10:1, at least 7:1, at least 5:1, at least 4:1, at least 3:1, at least 2:1, or at least 1:1. In some embodiments, the ratio of an mRNA comprising an open reading frame encoding a picornavirus 3C protease to a mRNA comprising an open reading frame encoding a picornavirus capsid polyprotein is 10:1. In some embodiments, the ratio of an mRNA comprising an open reading frame encoding a picornavirus 3C protease to a mRNA comprising an open reading frame encoding a picornavirus capsid polyprotein is 2:1.

In some embodiments, the polynucleotides encoding one or more picornavirus capsid polyprotein and/or picornavirus 3C protease thereof are present in a composition in a ratio such that the capsid proteins form a protomer. In some embodiments, the protomers form a pentamer. In some embodiments, the pentamers form a VLP.

Neutralizing antibodies can be produced against surface-exposed regions of viral particles. For example, in human rhinovirus (HRV), the viral protein VP1 is the most surface-exposed of the viral proteins and is therefore the most immunogenic viral protein. Neutralizing antibodies are often directed towards Neutralizing Immunogenic (NIm) sites on viral particles. The amino acid sequences of NIm sites are highly variable between viral serotypes. Although viral particles comprise highly-conserved regions, such highly-conserved regions are usually buried on the inside of the viral particle and are not accessible to neutralizing antibodies. In some embodiments of the present invention, the VLP comprises Neutralizing Immunogenic (NIm) sites.

In some embodiments, compositions of the present invention are formulated in at least one lipid nanoparticle. In some embodiments, compositions of the present invention are formulated in two lipid nanoparticles.

The polynucleotides and/or compositions of the present invention are useful in assembling VLPs that mimic virus or a viral particle and trigger an immunogenic response when administered to a subject. In some embodiments, the virus is a member of the Picornaviridae family of viruses.

The Picornaviridae family of viruses is classified into 29 genera, one of which is the Enterovirus genus. The Enterovirus genus is further classified into 12 species, including Enteroviruses A-D, Rhinoviruses A-C, and five Enterovirus species that only infect animal species (van der Linden et al., 2015). The Enterovirus species that infect humans (e.g., Enteroviruses A-D and Rhinoviruses A-C) result in infections that range in severity from a simple common cold to life-threatening disease.

Species of Rhinovirus are responsible for symptoms associated with the common cold. In some embodiments of the present invention, a Rhinovirus is Human rhinovirus type 16 (HRV-A16). In some embodiments, a Rhinovirus is a Human rhinovirus type 14 (HRV-B14).

Species of Enterovirus that infect humans are responsible for more severe symptoms and diseases. For example, Enterovirus A71 (EV-A71), a genotype of a virus from the Enterovirus A species, has been linked to polio-like symptoms affecting the central nervous system such as acute flaccid myelitis, resulting in limb paralysis. For another example, viruses from the Enterovirus B (e.g., Coxsackievirus B3) species have been linked to hand, foot and mouth disease. For yet another example, viruses from the Enterovirus C species have been linked to poliomyelitis (Polio disease). For yet another example, Enterovirus D68 (EV-D68), a genotype of a virus from the Enterovirus D species, has been linked to severe respiratory disease and/or acute flaccid myelitis. In some embodiments of the present invention, an Enterovirus is EV-A71. In some embodiments, an Enterovirus is EV-D68.

Examples of viruses which may be immunized against using the compositions or constructs of the present invention include, but are not limited to, members of the species Enterovirus A, formerly named Human Enterovirus A, (e.g., serotypes coxsackievirus A2 (CVA2), CVA3, CVA4, CVA5, CVA6, CVA7, CVA8, CVA10, CVA12, CVA14, CVA16, enterovirus A71 (EV-A71), EV-A76, EV-A89, EV-A90, EV-A91, EV-A92, EV-A114, EV-A119, EV-A120, EV-A121, EV-A122 (formerly SV19), EV-A123 (formerly SV43), EV-A124 (formerly SV46) and EV-125 (formerly BA13)); members of the species Enterovirus B, formerly named Human Enterovirus B (e.g., serotypes coxsackievirus B1 (CVB1), CVB2, CVB3, CVB4 (including swine vesicular disease virus 2 SVDV-2, CVB5 (including SVDV-1), CVB6, CVA9, echovirus 1 (E1; incl. E8), E2, E3, E4, E5, E6, E7, E9 (including CVA23) E11, E12, E13, E14, E15, E16, E17, E18, E19, E20, E21, E24, E25, E26, E27, E29, E30, E31, E32, E33, enterovirus B69 (EV-B69), EV-B73, EV-B74, EV-B75, EV-B77, EV-B78, EV-B79, EV-B80, EV-B81, EV-B82, EV-B83, EV-B84, EV-B85, EV-B86, EV-B87, EV-B88, EV-B93, EV-B97, EV-B98, EV-B100, EV-B101, EV-B106, EV-B107, EV-B110 (from a chimpanzee), EV-B111, EV-B112 (from a chimpanzee), EV-B113 (from a Mandrill) and EV-B114 (formerly named simian agent 5 (SA5)); members of the species Enterovirus C, formerly named Human Enterovirus C (e.g., poliovirus (PV) 1, PV2, PV3, coxsackievirus A1 (CVA1), CVA11, CVA13, CVA17, CVA19, CVA20, CVA21, CVA22, CVA24, EV-C95, EV-C96, EV-C99, EV-C102, EV-C104, EV-C105, EV-C109, EV-C113, EV-C116, EV-C117 and EV-C118); members of the species Enterovirus D, formerly named Human Enterovirus) (e.g., serotypes EV-D68, EV-D70, EV-D94, EV-D111 (from both humans & chimpanzees) and EV-D120 (from gorillas)); members of the species Rhinovirus A, formerly named Human rhinovirus A (e.g., serotypes rhinovirus (RV) A1, A2, A7, A8, A9, A10, A11, A12, A13, A15, A16, A18, A19, A20, A21, A22, A23, A24, A25, A28, A29, A30, A31, A32, A33, A34, A36, A38, A39, A40, A41, A43, A45, A46, A47, A49, A50, A51, A53, A54, A55, A56, A57, A58, A59, A60, A61, A62, A63, A64, A65, A66, A67, A68, A71, A73, A74, A75, A76, A77, A78, A80, A81, A82, A85, A88, A89, A90, A94, A96, A100, A101, A102, A103, A104, A105, A106, A107, A108, A109); members of the species Rhinovirus B, formerly named Human rhinovirus B (e.g., serotypes rhinovirus (RV) B3, B4, B5, B6, B14, B17, B26, B27, B35, B37, B42, B48, B52, B69, B70, B72, B79, B83, B84, B86, B91, B92, B93, B97, B99, B100, B101, B102, B103, B104, B105 & B106); and members of the species Rhinovirus C, formerly named Human rhinovirus C (e.g., serotype C1, C2, C3, C4, C5, C6, C7, C8, C9, C10, C11, C12, C13, C14, C15, C16, C17, C18, C19, C20, C21, C22, C23, C24, C25, C26, C27, C28, C29, C30, C31, C32, C33, C34, C35, C36, C37, C38, C39, C40, C41, C42, C43, C44, C45, C46, C47, C48, C49, C50, & C51).

The compositions of the present disclosure may be designed as a single prophylactic therapeutic that immunizes a subject against a variety of pathogenic strains of Enterovirus.

In some aspects, a method of the present disclosure comprises administering to a subject an immunogenic composition described herein. As used herein, an “immunogenic composition” refers to a composition comprising an mRNA comprising an open reading frame encoding a protease and an mRNA comprising an open reading frame encoding a capsid polyprotein comprising a precursor protein, wherein the precursor protein comprises two or more capsids and has a cleavage site specific for the protease between the two or more capsids, in an effective amount to induce in the subject an immune response against a viral infection from a member of the Enterovirus genus.

An immune response includes a binding antibody titer to a species of virus. In some embodiments, an immune response includes a binding antibody titer to a human rhinovirus species of virus (e.g., HRV-A16 or HRV-B14). In some embodiments, an immune response includes a binding antibody titer to a human enterovirus species of virus (e.g., EV-A71 or EV-D68).

An immune response further includes a neutralizing antibody titer to a species of virus. In some embodiments, an immune response includes a neutralizing antibody titer to a human rhinovirus species of virus (e.g., HRV-A16 or HRV-B14). In some embodiments, an immune response includes a neutralizing antibody titer to a human enterovirus species of virus (e.g., EV-A71 or EV-D68).

An immune response further includes a T cell response to a species of virus. In some embodiments, an immune response includes a T cell response to a human rhinovirus species of virus (e.g., HRV-A16 or HRV-B14). In some embodiments, an immune response includes a T cell response to a human enterovirus species of virus (e.g., EV-A71 or EV-D68).

According to the present invention, polynucleotides or constructs and their associated compositions may be designed to produce a commercially available vaccine, a variant or a portion thereof in vivo.

The polynucleotides of the invention may be used to protect an infant against infection or disease, including but not limited to diseases caused by viruses in the Picornaviridae family. Examples of diseases caused by viruses in the Picornaviridae family of the present invention, include but are not limited to aseptic meningitis (the most common acute viral disease of the CNS), encephalitis, upper respiratory tract illness, lower respiratory track illness, the common cold, febrile rash illnesses (hand-foot-and-mouth disease), conjunctivitis, herpangina, myositis and myocarditis, hepatitis, poliomyelitis, polio-like viral illness, acute flaccid myelitis (AFM), gastrointestinal disturbances and conjunctivitis.

In some embodiments, the polynucleotides of the invention may encode at least one picornavirus capsid polyprotein and/or picornavirus 3C protease that forms a VLP when administered to a subject and immunizes the subsection for the prevention, management, or treatment of Enterovirus infections.

In one embodiment, the polynucleotides of the present invention is or functions as a messenger RNA (mRNA). As used herein, the term “messenger RNA” (mRNA) refers to any polynucleotide which encodes at least one peptide or polypeptide of interest and which is capable of being translated to produce the encoded peptide polypeptide of interest in vitro, in vivo, in situ or ex vivo.

The basic components of an mRNA molecule typically include at least one coding region, a 5′ untranslated region (UTR), a 3′ UTR, a 5′ cap, and a poly-A tail. Polynucleotides of the present disclosure may function as mRNA but can be distinguished from wild-type mRNA in their functional and/or structural design features which serve to overcome existing problems of effective polypeptide expression using nucleic-acid based therapeutics.

Polynucleotides of the present disclosure, in some embodiments, are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park CA), and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence (e.g., a naturally-occurring or wild-type mRNA sequence encoding a polypeptide or protein of interest, such as a picornavirus capsid polyprotein and/or picornavirus 3C protease thereof). In some embodiments, a codon optimized sequence shares less than 90% sequence identity to a naturally-occurring or wild-type sequence (e.g., a naturally-occurring or wild-type mRNA sequence encoding a polypeptide or protein of interest, such as a picornavirus capsid polyprotein and/or picornavirus 3C protease thereof). In some embodiments, a codon optimized sequence shares less than 85% sequence identity to a naturally-occurring or wild-type sequence (e.g., a naturally-occurring or wild-type mRNA sequence encoding a polypeptide or protein of interest, such as a picornavirus capsid polyprotein and/or picornavirus 3C protease thereof). In some embodiments, a codon optimized sequence shares less than 80% sequence identity to a naturally-occurring or wild-type sequence (e.g., a naturally-occurring or wild-type mRNA sequence encoding a polypeptide or protein of interest, such as a picornavirus capsid polyprotein and/or picornavirus 3C protease thereof). In some embodiments, a codon optimized sequence shares less than 75% sequence identity to a naturally-occurring or wild-type sequence (e.g., a naturally-occurring or wild-type mRNA sequence encoding a polypeptide or protein of interest, such as a picornavirus capsid polyprotein and/or picornavirus 3C protease thereof).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a polypeptide or protein of interest, such as a picornavirus capsid polyprotein and/or picornavirus 3C protease thereof). In some embodiments, a codon optimized sequence shares between 65% and 75 or about 80% sequence identity to a naturally-occurring or wild-type sequence (e.g., a naturally-occurring or wild-type mRNA sequence encoding a polypeptide or protein of interest, such as a picornavirus capsid polyprotein and/or picornavirus 3C protease thereof).

In some embodiments a codon optimized RNA may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

Polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. Polypeptides may also comprise single chain or multichain polypeptides such as antibodies and may be associated or linked. Most commonly, disulfide linkages are found in multichain polypeptides. The term polypeptide may also apply to amino acid polymers in which at least one amino acid residue is an artificial chemical analogue of a corresponding naturally-occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants possess at least 50% identity to a native or reference sequence. In some embodiments, variants share at least 80%, or at least 90°/% identity with a native or reference sequence.

In some embodiments “variant mimics” are provided. As used herein, the term “variant mimic” is one which contains at least one amino acid that would mimic an activated sequence. For example, glutamate may serve as a mimic for phosphoro-threonine and/or phosphoro-serine. Alternatively, variant mimics may result in deactivation or in an inactivated product containing the mimic, for example, phenylalanine may act as an inactivating substitution for tyrosine; or alanine may act as an inactivating substitution for serine.

“Orthologs” refers to genes in different species that evolved from a common ancestral gene by speciation. Normally, orthologs retain the same function in the course of evolution. Identification of orthologs is critical for reliable prediction of gene function in newly sequenced genomes.

“Analogs” is meant to include polypeptide variants which differ by one or more amino acid alterations, for example, substitutions, additions or deletions of amino acid residues that still maintain one or more of the properties of the parent or starting polypeptide.

The present disclosure provides several types of compositions that are polynucleotide or polypeptide based, including variants and derivatives. These include, for example, substitutional, insertional, deletion and covalent variants and derivatives. The term “derivative” is used synonymously with the term “variant” but generally refers to a molecule that has been modified and/or changed in any way relative to a reference molecule or starting molecule.

As such, polynucleotides encoding peptides or polypeptides containing substitutions, insertions and/or additions, deletions and covalent modifications with respect to reference sequences, in particular the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art.

“Substitutional variants” when referring to polypeptides are those that have at least one amino acid residue in a native or starting sequence removed and a different amino acid inserted in its place at the same position. Substitutions may be single, where only one amino acid in the molecule has been substituted, or they may be multiple, where two or more amino acids have been substituted in the same molecule.

As used herein, the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Features” when referring to polypeptide or polynucleotide are defined as distinct amino acid sequence-based or nucleotide-based components of a molecule respectively. Features of the polypeptides encoded by the polynucleotides include surface manifestations, local conformational shape, folds, loops, half-loops, domains, half-domains, sites, termini, or any combination thereof.

As used herein, when referring to polypeptides, the term “domain” refers to a motif of a polypeptide having one or more identifiable structural or functional characteristics or properties (e.g., binding capacity, serving as a site for protein-protein interactions).

As used herein, when referring to polypeptides, the terms “site” as it pertains to amino acid based embodiments is used synonymously with “amino acid residue” and “amino acid side chain.” As used herein, when referring to polynucleotides, the terms “site” as it pertains to nucleotide based embodiments is used synonymously with “nucleotide.” A site represents a position within a peptide or polypeptide or polynucleotide that may be modified, manipulated, altered, derivatized or varied within the polypeptide or polynucleotide based molecules.

As used herein, the terms “termini” or “terminus” when referring to polypeptides or polynucleotides refers to an extremity of a polypeptide or polynucleotide, respectively. Such extremity is not limited only to the first or final site of the polypeptide or polynucleotide, but may include additional amino acids or nucleotides in the terminal regions. Polypeptide-based molecules may be characterized as having both an N-terminus (terminated by an amino acid with a free amino group (NH₂)) and a C-terminus (terminated by an amino acid with a free carboxyl group (COOH)). Proteins are in some cases made up of multiple polypeptide chains brought together by disulfide bonds or by non-covalent forces (e.g., multimers, oligomers). These proteins have multiple N- and C-termini. Alternatively, the termini of the polypeptides may be modified such that they begin or end, as the case may be, with a non-polypeptide based moiety such as an organic conjugate.

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length. In another example, any protein that includes a stretch of 20, 30, 40, 50, or 100 amino acids which are 40%, 50%, 60%, 70%, 80%, 90%, 95%, or 100% identical to any of the sequences described herein can be utilized in accordance with the disclosure. In some embodiments, a polypeptide includes 2, 3, 4, 5, 6, 7, 8, 9, 10, or more mutations as shown in any of the sequences provided or referenced herein. In another example, any protein that includes a stretch of 20, 30, 40, 50, or 100 amino acids that are greater than 80%, 90%, 95%, or 100% identical to any of the sequences described herein, wherein the protein has a stretch of 5, 10, 15, 20, 25, or 30 amino acids that are less than 80%, 75%, 70%, 65% or 60% identical to any of the sequences described herein can be utilized in accordance with the disclosure.

Polypeptide or polynucleotide molecules of the present disclosure may share a certain degree of sequence similarity or identity with the reference molecules (e.g., reference polypeptides or reference polynucleotides), for example, with art-described molecules (e.g., engineered or designed molecules or wild-type molecules). The term “identity” as known in the art, refers to a relationship between the sequences of two or more polypeptides or polynucleotides, as determined by comparing the sequences. In the art, identity also means the degree of sequence relatedness between them as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453.). More recently, a Fast Optimal Global Sequence Alignment Algorithm (FOGSAA) has been developed that purportedly produces global alignment of nucleotide and protein sequences faster than other optimal global alignment methods, including the Needleman-Wunsch algorithm. Other tools are described herein, specifically in the definition of “identity” below.

As used herein, the term “homology” refers to the overall relatedness between polymeric molecules, e.g. between nucleic acid molecules (e.g., DNA molecules and/or RNA molecules) and/or between polypeptide molecules. Polymeric molecules (e.g., nucleic acid molecules (e.g., DNA molecules and/or RNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original one.

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

RNA (e.g., mRNA) treatments of the present disclosure may comprise at least one ribonucleic acid (RNA) polynucleotide having an open reading frame encoding a picornavirus capsid polyprotein that comprises at least one chemical modification. RNA (e.g., mRNA) treatments of the present disclosure may comprise at least one ribonucleic acid (RNA) polynucleotide having an open reading frame encoding a picornavirus 3C protease that comprises at least one chemical modification.

The terms “chemical modification” and “chemically modified” refer to modification with respect to adenosine (A), guanosine (G), uridine (U), thymidine (T) or cytidine (C) ribonucleosides or deoxyribonucleosides in at least one of their position, pattern, percent or population. Generally, these terms do not refer to the ribonucleotide modifications in naturally occurring 5′-terminal mRNA cap moieties. With respect to a polypeptide, the term “modification” refers to a modification relative to the canonical set 20 amino acids. Polypeptides, as provided herein, are also considered “modified” if they contain amino acid substitutions, insertions, or a combination of substitutions and insertions.

Polynucleotides (e.g., RNA polynucleotides, such as mRNA polynucleotides), in some embodiments, comprise various (more than one) different modifications. In some embodiments, a particular region of a polynucleotide contains one, two or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Modifications of polynucleotides include, without limitation, those described herein. Polynucleotides (e.g., RNA polynucleotides, such as mRNA polynucleotides) may comprise modifications that are naturally-occurring, non-naturally-occurring, or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone).

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

The present disclosure provides for modified nucleosides and nucleotides of a polynucleotide (e.g., RNA polynucleotides, such as mRNA polynucleotides). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside comprising one or more phosphate groups. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine, or uracil. Any combination of base/sugar or linker may be incorporated into polynucleotides of the present disclosure.

The skilled artisan will appreciate that, except where otherwise noted, polynucleotide sequences set forth in the instant application will recite “T”s in a representative DNA sequence but where the sequence represents RNA, the “T”s would be substituted for “U”s.

Modifications of polynucleotides (e.g., RNA polynucleotides, such as mRNA polynucleotides) that are useful in the compositions, methods and synthetic processes of the present disclosure of the present disclosure include, but are not limited to the following: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; I-methyladenosine; 2′-O-methyladenosine; 2′-O-ribosyladenosine (phosphate); 2-methyladenosine; 2-methylthio-N6 Isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6, N6 (dimethyl)adenine; N6-cis-hydroxy-isopentenyl-adenosine; α-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′-Fluoro-N6-Bz-deoxyadenosine TP; 2′-OMe-2-Amino-ATP; 2′-O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; α-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine; 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(I-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; α-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl)uridine; 1-methyl-3-(3-amino-5-carboxypropyl)pseudouridine; 1-methylpseduouridine; 1-methyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester; 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methyluridine), 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine, N1-ethylpseudouridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; α-thio-uridine; 1 (aminoalkylamino-carbonylethylenyl)-2(thio)-pseudouracil; 1 (aminoalkylaminocarbonylethylenyl)-2,4-(dithio)pseudouracil; 1 (aminoalkylaminocarbonylethylenyl)-4 (thio)pseudouracil; 1 (aminoalkylaminocarbonylethylenyl)-pseudouracil; 1 (aminocarbonylethylenyl)-2(thio)-pseudouracil; 1 (aminocarbonylethylenyl)-2,4-(dithio)pseudouracil; 1 (aminocarbonylethylenyl)-4 (thio)pseudouracil; 1 (aminocarbonylethylenyl)-pseudouracil; 1 substituted 2(thio)-pseudouracil; 1 substituted 2,4-(dithio)pseudouracil; 1 substituted 4 (thio)pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP; 1-Methyl-3-(3-amino-3-carboxypropyl)pseudo-UTP; 1-Methyl-pseudo-UTP; 2 (thio)pseudouracil; 2′ deoxy uridine; 2′ fluorouridine; 2-(thio)uracil; 2,4-(dithio)pseudouracil; 2′ methyl, 2′amino, 2′azido, 2′fluoroguanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouracil; 4-(thio)pseudouracil; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio)pseudouracil; 5-(alkyl)pseudouracil; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouracil; 5-(methyl)2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; Pseudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (±)1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-45-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouridine TP; 1-{3-[2-(2-Aminoethoxy)-ethoxy]-propionyl}pseudouridine TP; 1-Acetylpseudouridine TP; 1-Alkyl-6-(1-propynyl)-pseudo-UTP; 1-Alkyl-6-(2-propynyl)-pseudo-UTP: 1-Alkyl-6-allyl-pseudo-UTP: 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallyipseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(I-propynyl)uridine TP; 3-Alkyl-pseudo-UTP; 4′-Azidouridine TP: 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2(2-ethoxy)-ethoxy}-ethoxy]-ethoxy)-ethoxy}]propionic acid; Pseudouridine TP 1-[3-{2-(2-[2-ethoxy]-ethoxy)-ethoxy)]propionic acid; Pseudouridine TP 1-[3-(2-(2-ethoxy)-ethoxy)] propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino)purine; 2,4,5-(trimethyl)phenyl; 2′ methyl, 2′amino, 2′azido, 2′fluro-cytidine; 2′ methyl, 2′amino, 2′azido, 2′fluro-adenine; 2′methyl, 2′amino, 2′azido, 2′fluro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl: 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; O6-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; and N6-(19-Amino-pentaoxanonadecyl)adenosine TP.

In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of pseudouridine (ψ), N1-methylpseudouridine (m1ψ), N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine), 5-methoxyuridine and 2′-O-methyl uridine. In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of 1-methyl-pseudouridine (m1ψ), 5-methoxy-uridine (mo5U), 5-methyl-cytidine (m5C), pseudouridine (ψ), α-thio-guanosine, and α-thio-adenosine. In some embodiments, polynucleotides includes a combination of at least two (e.g., 2, 3, 4 or more) of the aforementioned modified nucleobases.

In some embodiments, polynucleotides (e.g., RNA polynucleotides, such as mRNA polynucleotides) comprise pseudouridine (ψ) and 5-methyl-cytidine (m5C). In some embodiments, polynucleotides (e.g., RNA polynucleotides, such as mRNA polynucleotides) comprise 1-methyl-pseudouridine (m1ψ). In some embodiments, polynucleotides (e.g., RNA polynucleotides, such as mRNA polynucleotides) comprise 1-methyl-pseudouridine (m1ψ) and 5-methyl-cytidine (m5C). In some embodiments, polynucleotides (e.g., RNA polynucleotides, such as mRNA polynucleotides) comprise 2-thiouridine (s2U). In some embodiments, polynucleotides (e.g., RNA polynucleotides, such as mRNA polynucleotides) comprise 2-thiouridine and 5-methyl-cytidine (m5C). In some embodiments, polynucleotides (e.g., RNA polynucleotides, such as mRNA polynucleotides) comprise methoxy-uridine (mo5U). In some embodiments, polynucleotides (e.g., RNA polynucleotides, such as mRNA polynucleotides) comprise 5-methoxy-uridine (mo5U) and 5-methyl-cytidine (m5C). In some embodiments, polynucleotides (e.g., RNA polynucleotides, such as mRNA polynucleotides) comprise 2′-O-methyl uridine. In some embodiments polynucleotides (e.g., RNA polynucleotides, such as mRNA polynucleotides) comprise 2′-O-methyl uridine and 5-methyl-cytidine (m5C). In some embodiments, polynucleotides (e.g., RNA polynucleotides, such as mRNA polynucleotides) comprise N6-methyl-adenosine (m6A). In some embodiments, polynucleotides (e.g., RNA polynucleotides, such as mRNA polynucleotides) comprise N6-methyl-adenosine (m6A) and 5-methyl-cytidine (m5C).

In some embodiments, polynucleotides (e.g., RNA polynucleotides, such as mRNA polynucleotides) are uniformly modified (e.g., fully modified, modified throughout the entire sequence) for a particular modification. For example, a polynucleotide can be uniformly modified with 5-methyl-cytidine (m5C), meaning that all cytosine residues in the mRNA sequence are replaced with 5-methyl-cytidine (m5C). Similarly, a polynucleotide can be uniformly modified for any type of nucleoside residue present in the sequence by replacement with a modified residue such as those set forth above.

Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine.

In some embodiments, a modified nucleobase is a modified uridine. Exemplary nucleobases and nucleosides having a modified uridine include 5-cyano uridine, and 4′-thio uridine.

In some embodiments, a modified nucleobase is a modified adenine. Exemplary nucleobases and nucleosides having a modified adenine include 7-deaza-adenine, 1-methyl-adenosine (m1A), 2-methyl-adenine (m2A), and N6-methyl-adenosine (m6A).

In some embodiments, a modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQO), 7-aminomethyl-7-deaza-guanosine (preQ1), 7-methyl-guanosine (m7G), 1-methyl-guanosine (m1G), 8-oxo-guanosine, 7-methyl-8-oxo-guanosine.

The polynucleotides of the present disclosure may be partially or fully modified along the entire length of the molecule. For example, one or more or all or a given type of nucleotide (e.g., purine or pyrimidine, or any one or more or all of A, G, U, C) may be uniformly modified in a polynucleotide of the disclosure, or in a given predetermined sequence region thereof (e.g., in the mRNA including or excluding the polyA tail). In some embodiments, all nucleotides X in a polynucleotide of the present disclosure (or in a given sequence region thereof) are modified nucleotides, wherein X may any one of nucleotides A, G, U, C, or any one of the combinations A+G, A+U, A+C, G+U, G+C, U+C, A+G+U, A+G+C, G+U+C, or A+G+C.

The polynucleotide may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 500%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 500% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The polynucleotides may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the polynucleotides may contain a modified pyrimidine such as a modified uracil or cytosine. In some embodiments, at least 5%, at least 10%, at least 25%, at least 50%, at least 80%, at least 90%, or 100% of the uracil in the polynucleotide is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90%, or 100% of the cytosine in the polynucleotide is replaced with a modified cytosine (e.g., a 5-substituted cytosine). The modified cytosine can be replaced by a compound having a single unique structure or can be replaced by a plurality of compounds having different structures (e.g., 2, 3, 4 or more unique structures).

Thus, in some embodiments, the RNA treatments comprise a 5′UTR element, an optionally codon optimized open reading frame, and a 3′UTR element, a poly(A) sequence and/or a polyadenylation signal wherein the RNA is not chemically modified.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (im5U), 1-taurinomethyl-pseudouridine, 5-taurinomethyl-2-thio-uridine (rm5s2U), 1-taurinomethyl-4-thio-pseudouridine, 5-methyl-uridine (m5U, i.e., having the nucleobase deoxythymine), 1-methyl-pseudouridine (m1ψ), 5-methyl-2-thio-uridine (m5s2U), 1-methyl-4-thio-pseudouridine (m1s4ψ), 4-thio-1-methyl-pseudouridine, 3-methyl-pseudouridine (m3W), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, N1-ethylpseudouridine, 3-(3-amino-3-carboxypropyl)uridine (acp3U), 1-methyl-3-(3-amino-3-carboxypropyl)pseudouridine (acp3% ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (Wm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-0-trimethyl-cytidine (m42Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl)adenosine (io6A), 2-methylthio-N6-(cis-hydroxyisopentenyl)adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, α-thio-adenosine, 2′-O-methyl-adenosine (Am), N6,2′-O-dimethyl-adenosine (m6Am), N6,N6,2′-O-trimethyladenosine (m62Am), 1,2′-O-dimethyl-adenosine (m1Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQO), 7-aminomethyl-7-deaza-guanosine (preQ1), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine (m1G), N2-methyl-guanosine (m2G), N2,N2-dimethyl-guanosine (m22G), N2,7-dimethyl-guanosine (m2,7G), N2, N2,7-dimethyl-guanosine (m2,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 2′-O-ribosylguanosine (phosphate) (Gr(p)), 1-thio-guanosine, O6-methyl-guanosine, 2′-F-ara-guanosine, and 2′-F-guanosine.

Antibodies and antigen binding fragments thereof of the present disclosure comprise at least one RNA polynucleotide, such as an mRNA (e.g., modified mRNA). mRNA, for example, is transcribed in vitro from template DNA, referred to as an “in vitro transcription template.” In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the mRNA encoded by the template.

### Sequence Optimization

In some embodiments, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites: adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding a picornavirus capsid polyprotein and/or picornavirus 3C protease). In some embodiments, a codon optimized sequence shares less than 90% sequence identity to a naturally-occurring or wild-type sequence (e.g., a naturally-occurring or wild-type mRNA sequence encoding a picornavirus capsid polyprotein and/or picornavirus 3C protease). In some embodiments, a codon optimized sequence shares less than 85% sequence identity to a naturally-occurring or wild-type sequence (e.g., a naturally-occurring or wild-type mRNA sequence encoding a picornavirus capsid polyprotein and/or picornavirus 3C protease). In some embodiments, a codon optimized sequence shares less than 80% sequence identity to a naturally-occurring or wild-type sequence (e.g., a naturally-occurring or wild-type mRNA sequence encoding a picornavirus capsid polyprotein and/or picornavirus 3C protease). In some embodiments, a codon optimized sequence shares less than 75% sequence identity to a naturally-occurring or wild-type sequence (e.g., a naturally-occurring or wild-type mRNA sequence encoding a picornavirus capsid polyprotein and/or picornavirus 3C protease).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a picornavirus capsid polyprotein and/or picornavirus 3C protease). In some embodiments, a codon optimized sequence shares between 65% and 75% or about 80% sequence identity to a naturally-occurring or wild-type sequence (e.g., a naturally-occurring or wild-type mRNA sequence encoding a picornavirus capsid polyprotein and/or picornavirus 3C protease).

In some embodiments, a codon-optimized sequence encodes a picornavirus capsid polyprotein and/or picornavirus 3C protease that is as immunogenic as, or more immunogenic than (e.g., at least 10%, at least 20%, at least 30%, at least 40%, at least 50%, at least 100%, or at least 200% more), than a picornavirus capsid polyprotein and/or picornavirus 3C protease encoded by a non-codon-optimized sequence.

When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemically Unmodified Nucleotides

In some embodiments, an RNA (e.g., mRNA) is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

### Chemical Modifications

The compositions of the present disclosure comprise, in some embodiments, an RNA having an open reading frame encoding a coronavirus antigen, wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published US application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB2017/051367 all of which are incorporated by reference herein.

Hence, nucleic acids of the disclosure (e.g., DNA nucleic acids and RNA nucleic acids, such as mRNA nucleic acids) can comprise standard nucleotides and nucleosides, naturally-occurring nucleotides and nucleosides, non-naturally-occurring nucleotides and nucleosides, or any combination thereof.

Nucleic acids of the disclosure (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise 1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a mRNA of the disclosure comprises 1-methyl-pseudouridine (m1ψ) substitutions at one or more or all uridine positions of the nucleic acid.

In some embodiments, a mRNA of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a mRNA of the disclosure comprises pseudouridine (ψ) substitutions at one or more or all uridine positions of the nucleic acid.

In some embodiments, a mRNA of the disclosure comprises pseudouridine (ψ) substitutions at one or more or all uridine positions of the nucleic acid and 5-methyl cytidine substitutions at one or more or all cytidine positions of the nucleic acid.

In some embodiments, a mRNA of the disclosure comprises uridine at one or more or all uridine positions of the nucleic acid.

In some embodiments, mRNAs are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the poly(A) tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The mRNAs may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Untranslated Regions (UTRs)

The mRNAs of the present disclosure may comprise one or more regions or parts which act or function as an untranslated region. Where mRNAs are designed to encode at least one antigen of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas, the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding) Natural 5′UTRs have features that play roles in translation initiation. They harbor signatures like Kozak sequences which are commonly known to be involved in the process by which the ribosome initiates translation of many genes. Kozak sequences have the consensus CCR(A/G)CCAUGG (SEQ ID NO: 27), where R is a purine (adenine or guanine) three bases upstream of the start codon (AUG), which is followed by another ‘G’0.5′UTR also have been known to form secondary structures which are involved in elongation factor binding.

In some embodiments of the disclosure, a 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219). CMV immediate-early 1 (IE1) gene (US20140206753, WO2013/185069), the sequence GGGAUCCUACC (SEQ ID NO: 28) (WO2014144196) may also be used. In another embodiment, 5′ UTR of a TOP gene is a 5′ UTR of a TOP gene lacking the 5′ TOP motif (the oligopyrimidine tract) (e.g., WO/2015101414, WO2015101415, WO/2015/062738, WO2015024667, WO2015024667; 5′ UTR element derived from ribosomal protein Large 32 (L32) gene (WO/2015101414, WO2015101415, WO/2015/062738), 5′ UTR element derived from the 5′UTR of an hydroxysteroid (17-β) dehydrogenase 4 gene (HSD17B4) (WO2015024667), or a 5′ UTR element derived from the 5′ UTR of ATP5A1 (WO2015024667) can be used. In some embodiments, an internal ribosome entry site (IRES) is used instead of a 5′ UTR.

In some embodiments, a 5′ UTR of the present disclosure comprises SEQ ID NO: 2.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/AXU/A) nonamers. Molecules containing this type of AREs include GM-CSF and TNF-a. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of nucleic acids (e.g., RNA) of the disclosure. When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

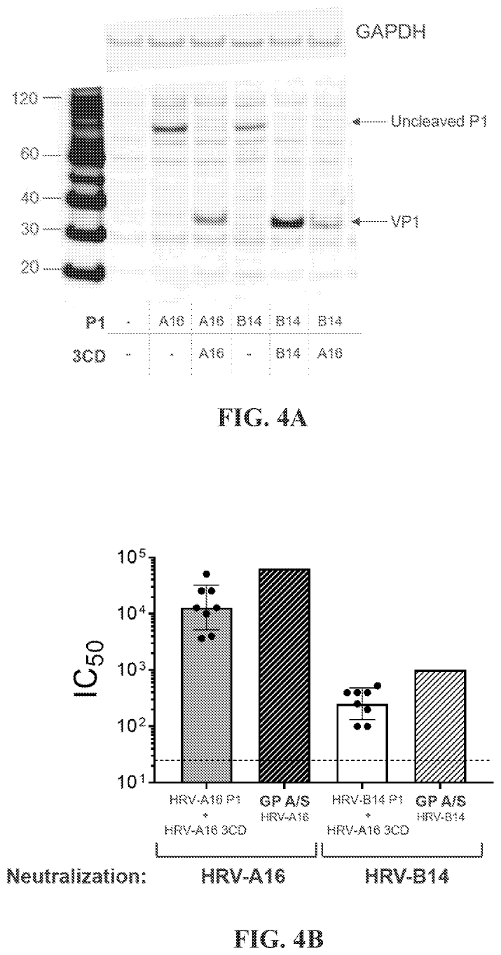

3′ UTRs may be heterologous or synthetic. With respect to 3′ UTRs, globin UTRs, including Xenopus β-globin UTRs and human β-globin UTRs are known in the art (U.S. Pat. Nos. 8,278,063, 9,012,219, US20110086907). A modified β-globin construct with enhanced stability in some cell types by cloning two sequential human β-globin 3′UTRs head to tail has been developed and is well known in the art (US2012/0195936, WO2014/071963). In addition a2-globin, a1-globin, UTRs and mutants thereof are also known in the art (WO2015101415, WO2015024667). Other 3′ UTRs described in the mRNA constructs in the non-patent literature include CYBA (Ferizi et al., 2015) and albumin (Thess et al., 2015). Other exemplary 3′ UTRs include that of bovine or human growth hormone (wild type or modified) (WO2013/185069, US20140206753, WO2014152774), rabbit β globin and hepatitis B virus (HBV), α-globin 3′ UTR and Viral VEEV 3′ UTR sequences are also known in the art. In some embodiments, the sequence UUUGAAUU (WO2014144196) is used. In some embodiments, 3′ UTRs of human and mouse ribosomal protein are used. Other examples include rps9 3′UTR (WO2015101414), FIG. 4 (WO2015101415), and human albumin 7 (WO2015101415).

In some embodiments, a 3′ UTR of the present disclosure comprises SEQ ID NO: 4.

Those of ordinary skill in the art will understand that 5′UTRs that are heterologous or synthetic may be used with any desired 3′ UTR sequence. For example, a heterologous 5′UTR may be used with a synthetic 3′UTR with a heterologous 3″ UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No. 20100293625 and PCT/US2014/069155, herein incorporated by reference in its entirety.

It should be understood that any UTR from any gene may be incorporated into the regions of a nucleic acid. Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 20100129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in US Application No. 20090226470, herein incorporated by reference in its entirety, and those known in the art.

### In Vitro Transcription of RNA

cDNA encoding the polynucleotides described herein may be transcribed using an in vitro transcription (IVT) system. In vitro transcription of RNA is known in the art and is described in International Publication WO 2014/152027, which is incorporated by reference herein in its entirety. In some embodiments, the RNA of the present disclosure is prepared in accordance with any one or more of the methods described in WO 2018/053209 and WO 2019/036682, each of which is incorporated by reference herein.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of a RNA polynucleotide, for example, but not limited to coronavirus mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes a RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a poly(A) tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “poly(A) tail” is a region of mRNA that is downstream, e.g., directly downstream (i.e., 3′), from the 3′ UTR that contains multiple, consecutive adenosine monophosphates. A poly(A) tail may contain 10 to 300 adenosine monophosphates. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly(A) tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a nucleic acid includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the RNA comprises 5′ terminal cap, for example, 7mG(5′)ppp(5′)NlmpNp.

### Chemical Synthesis

Solid-phase chemical synthesis. Nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

Liquid Phase Chemical Synthesis. The synthesis of nucleic acids of the present disclosure by the sequential addition of monomer building blocks may be carried out in a liquid phase.

Combination of Synthetic Methods. The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNATM oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the nucleic acids of the present disclosure may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Lipid Nanoparticle Formulations

In some embodiments, the RNA (e.g., mRNA) of the disclosure is formulated in a lipid nanoparticle (LNP). This is referred to herein as a LNP-formulated mRNA vaccine (fLNP). As used herein, “lipid nanoparticles” typically comprise ionizable cationic lipid usually an ionizable amino lipid, non-cationic lipid, sterol and PEG lipid components along with the nucleic acid cargo of interest. The lipid nanoparticles of the invention can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575; PCT/US2016/069491; PCT/US2016/069493; and PCT/US2014/066242, all of which are incorporated by reference herein in their entirety.

Vaccines of the present disclosure are typically formulated in lipid nanoparticle. In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises a molar ratio of 20-60% amino lipid relative to the other lipid components. For example, the lipid nanoparticle may comprise a molar ratio of 20-50%, 20-40%, 20-30%, 30-60%, 30-50%, 30-40%, 40-60%, 40-50%, or 50-60% amino lipid. In some embodiments, the lipid nanoparticle comprises a molar ratio of 20%, 30%, 40%, 50, or 60% amino lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20/o, 10-25%, 15-25%, 15-20%, 20-25%, or 25-30% phospholipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 3545%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises a molar ratio of 20-60/a amino lipid, 5-25% phospholipid, 25-55% structural lipid, and 0.5-15% PEG lipid.

In some embodiments, the lipid nanoparticle comprises a molar ratio of 20-60% amino lipid, 5-30% phospholipid, 10-55% structural lipid, and 0.5-15% PEG lipid.

### Amino Lipids

In some aspects, the amino lipids of the present disclosure may be one or more of compounds of Formula (I):

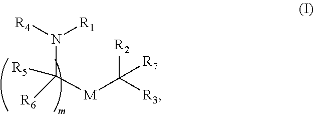

or their N-oxides, or salts or isomers thereof, wherein:
R₁ is selected from the group consisting of C₅₋₃₀ alkyl, C₁₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of hydrogen, a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —N(R)S(O)₂R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13; and wherein when R₄ is —(CH₂)ₘQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In certain embodiments, a subset of compounds of Formula (I) includes those of Formula (I-A):

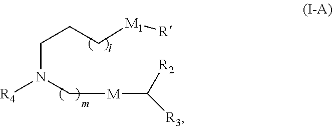

or its N-oxide, or a salt or isomer thereof, wherein I is selected from 1, 2, 3, 4, and 5; m is selected from 5, 6, 7, 8, and 9; M₁ is a bond or M′; R₄ is hydrogen, unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula

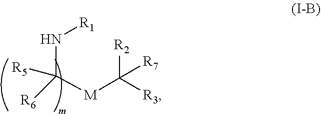

or its N-oxide, or a salt or isomer thereof in which all variables are as defined herein. For example, m is selected from 5, 6, 7, 8, and 9; R₄ is hydrogen, unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (II):

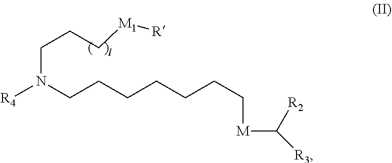

or its N-oxide, or a salt or isomer thereof, wherein I is selected from 1, 2, 3, 4, and 5; M₁ is a bond or M′; R₄ is hydrogen, unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In one embodiment, the compounds of Formula (I) are of Formula (IIa),

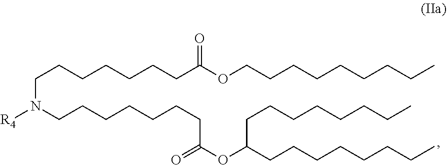

or their N-oxides, or salts or isomers thereof, wherein R₄ is as described herein.

In another embodiment, the compounds of Formula (I) are of Formula (IIb),

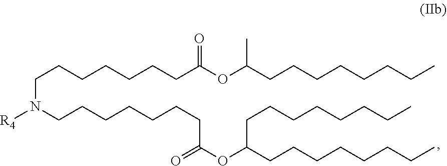

or their N-oxides, or salts or isomers thereof, wherein R₄ is as described herein.

In another embodiment, the compounds of Formula (I) are of Formula (IIc) or (IIe):

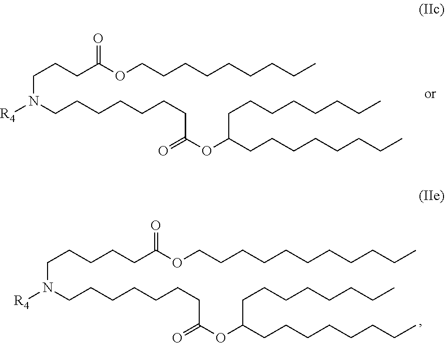

or their N-oxides, or salts or isomers thereof, wherein R₄ is as described herein.

In another embodiment, the compounds of Formula (I) are of Formula (IIf):

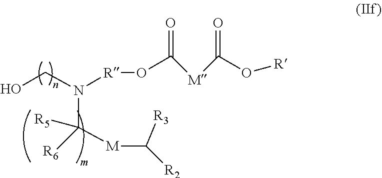

or their N-oxides, or salts or isomers thereof, wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (I) are of Formula (IId),

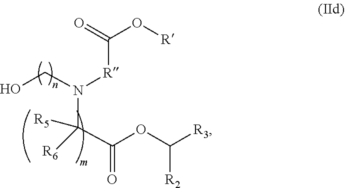

or their N-oxides, or salts or isomers thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In a further embodiment, the compounds of Formula (I) are of Formula (IIg),

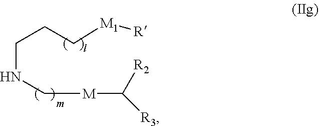

or their N-oxides, or salts or isomers thereof, wherein 1 is selected from 1, 2, 3, 4, and 5; m is selected from 5, 6, 7, 8, and 9: M₁ is a bond or M′; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

In some embodiments, the amino lipid is Compound 1

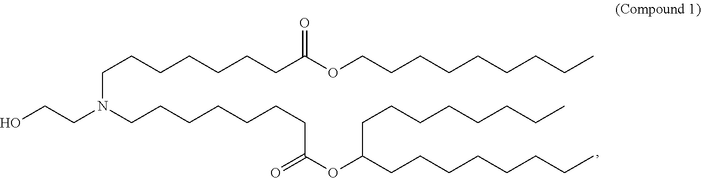 [SMILES: CCCCCCCCCOC(=O)CCCCCCCN(CCO)CCCCCCCC(=O)OC(CCCCCCCC)CCCCCCCC]

or a salt thereof.

In some embodiments, the amino lipid is Compound 2:

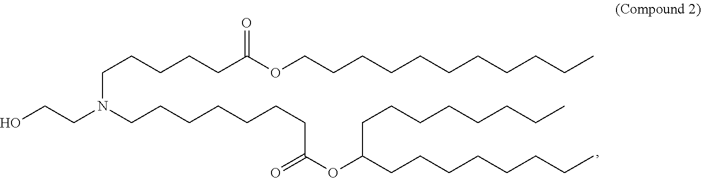 [SMILES: CCCCCCCCCCCOC(=O)CCCCCN(CCO)CCCCCCCC(=O)OC(CCCCCCCC)CCCCCCCC]

or a salt thereof.

The central amine moiety of a lipid according to Formula (I), (I-A), (I-B), (II), (IIa), (IIb), (IIc), (IId), (IIe), (IIf), or (IIg) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

In some aspects, the amino lipids of the present disclosure may be one or more of compounds of formula (III),

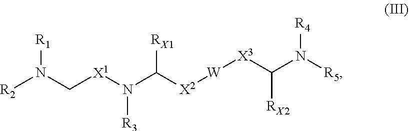

or salts or isomers thereof, wherein
W is

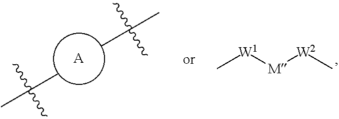

ring A is

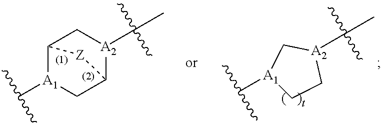

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR′;
R_{X1} and R_{X2} are each independently H or C₁₋₃ alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group,
M* is C₁-C₆ alkyl,
W¹ and W² are each independently selected from the group consisting of —O— and —N(R₆)—;
each R₆ is independently selected from the group consisting of H and C₁₋₅ alkyl;
X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6;
wherein when ring A is

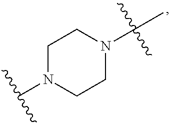 [SMILES: *N1CCN(*)CC1]

then
i) at least one of X¹, X², and X³ is not —CH₂—; and/or
ii) at least one of R₁, R₂, R₃, R₄, and R₅ is —R″MR′.

In some embodiments, the compound is of an of formulae (IIIa1)-(IIIa8):

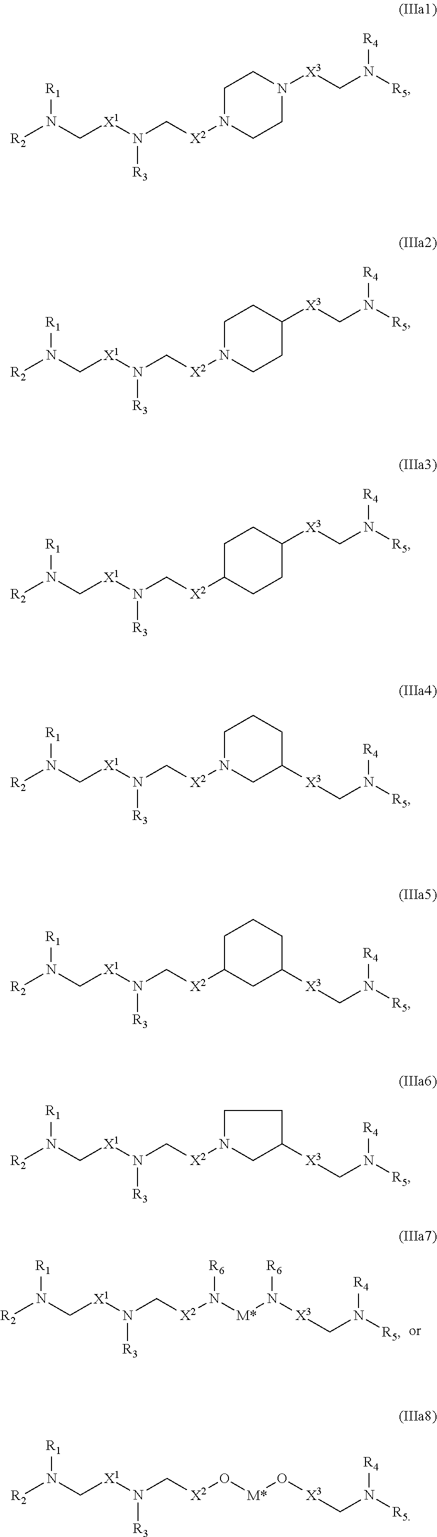

In some embodiments, the amino lipid is

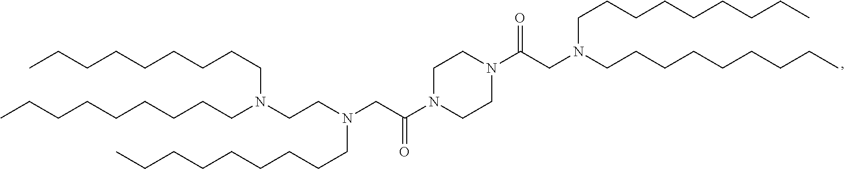 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (III), (IIIa1), (IIIa2), (IIIa3), (IIIa4), (IIIa5), (IIIa6), (IIIa7), or (IIIa8) may be protonated at a physiological pH. Thus, a lipid may have a positive or partial positive charge at physiological pH.

Phospholipids:

The lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present invention is an analog or variant of DSPC. In certain embodiments, a phospholipid useful or potentially useful in the present invention is a compound of Formula (IV):

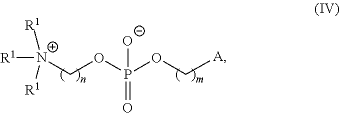

or a salt thereof, wherein:
each R₁ is independently optionally substituted alkyl; or optionally two R′ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

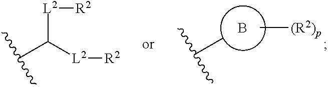

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the formula:

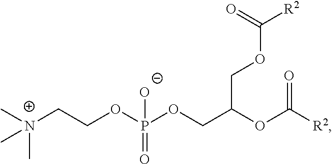

wherein each instance of R² is independently unsubstituted alkyl, unsubstituted alkenyl, or unsubstituted alkynyl.

In some embodiments, the phospholipids may be one or more of the phospholipids described in PCT Application No. PCT/US2018/037922.

Structural Lipids

The lipid composition of a pharmaceutical composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

Polyethylene Glycol (PEG)-Lipids

The lipid composition of a pharmaceutical composition disclosed herein can comprise one or more polyethylene glycol (PEG) lipids.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

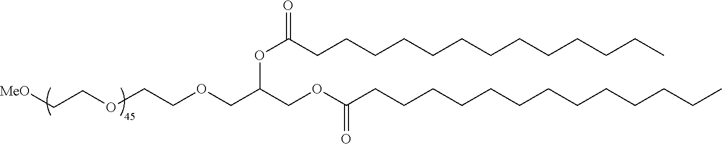

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (II). Provided herein are compounds of Formula (V):

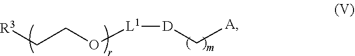

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10;
A is of the formula:

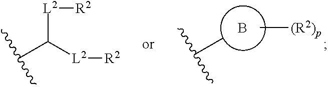

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (V) is a PEG-OH lipid (i.e., R³ is —OR^{O}, and R^{O} is hydrogen). In certain embodiments, the compound of Formula (V) is of Formula (V—OH):

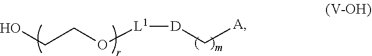

or a salt thereof.

In certain embodiments, a PEG lipid useful in the present invention is a PEGylated fatty acid. In certain embodiments, a PEG lipid useful in the present invention is a compound of Formula (VI). Provided herein are compounds of Formula (VI):

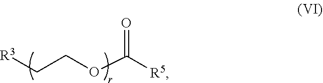

or a salts thereof, wherein:
R³ is-OR^{O}—
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}) NR^{N}C(═NR^{N})N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O; and
each instance of R^{N} is independently hydrogen, optionally substituted alkyl, or a nitrogen protecting group.

In certain embodiments, the compound of Formula (VI) is of Formula (VI-OH):

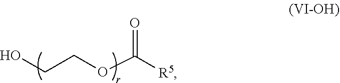

or a salt thereof. In some embodiments, r is 40-50.

In yet other embodiments the compound of Formula (VI) is:

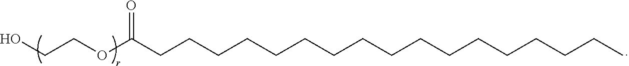

or a salt thereof.

In one embodiment, the compound of Formula (VI) is

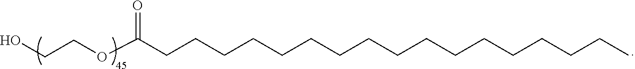

In some aspects, the lipid composition of the pharmaceutical compositions disclosed herein does not comprise a PEG-lipid.

In some embodiments, the PEG-lipids may be one or more of the PEG lipids described in U.S. Application No. U.S. Ser. No. 15/674,872.

In some embodiments, a LNP of the invention comprises an amino lipid of any of Formula I, II or III, a phospholipid comprising DSPC, a structural lipid, and a PEG lipid comprising PEG-DMG.

In some embodiments, a LNP of the invention comprises an amino lipid of any of Formula I, II or III, a phospholipid comprising DSPC, a structural lipid, and a PEG lipid comprising a compound having Formula VI.

In some embodiments, a LNP of the invention comprises an amino lipid of Formula I, II or III, a phospholipid comprising a compound having Formula IV, a structural lipid, and the PEG lipid comprising a compound having Formula V or VI.

In some embodiments, a LNP of the invention comprises an amino lipid of Formula I, II or III, a phospholipid comprising a compound having Formula IV, a structural lipid, and the PEG lipid comprising a compound having Formula V or VI.

In some embodiments, a LNP of the invention comprises an amino lipid of Formula I, II or III, a phospholipid having Formula IV, a structural lipid, and a PEG lipid comprising a compound having Formula VI.

In some embodiments, a LNP of the invention comprises an N:P ratio of from about 2:1 to about 30:1.

In some embodiments, a LNP of the invention comprises an N:P ratio of about 6:1.

In some embodiments, a LNP of the invention comprises an N:P ratio of about 3:1, 4:1, or 5:1.

In some embodiments, a LNP of the invention comprises a wt/wt ratio of the amino lipid component to the RNA of from about 10:1 to about 100:1.

In some embodiments, a LNP of the invention comprises a wt/wt ratio of the amino lipid component to the RNA of about 20:1.

In some embodiments, a LNP of the invention comprises a wt/wt ratio of the amino lipid component to the RNA of about 10:1.

In some embodiments, a LNP of the invention has a mean diameter from about 30 nm to about 150 nm.

In some embodiments, a LNP of the invention has a mean diameter from about 60 nm to about 120 nm.

As disclosed herein, a lipid nanoparticle (LNP) refers to a nanoscale construct (e.g., a nanoparticle, typically less than 100 nm in diameter) comprising lipid molecules arranged in a substantially spherical (i.e., spheroid) geometry, sometimes encapsulating one or more additional molecular species. A LNP may comprise or one or more types of lipids, including but not limited to amino lipids (e.g., ionizable amino lipids), neutral lipids, non-cationic lipids, charged lipids, PEG-modified lipids, phospholipids, structural lipids and sterols. In some embodiments, a LNP may further comprise one or more cargo molecules, including but not limited to nucleic acids (e.g., mRNA, plasmid DNA, DNA or RNA oligonucleotides, siRNA, shRNA, snRNA, snoRNA, lncRNA, etc.), small molecules, proteins and peptides. A LNP may have a unilamellar structure (i.e., having a single lipid layer or lipid bilayer surrounding a central region) or a multilamellar structure (i.e., having more than one lipid layer or lipid bilayer surrounding a central region). In some embodiments, a lipid nanoparticle may be a liposome. A liposome is a nanoparticle comprising lipids arranged into one or more concentric lipid bilayers around a central region. The central region of a liposome may comprise an aqueous solution, suspension, or other aqueous composition.

In some embodiments, a lipid nanoparticle may comprise two or more components (e.g., amino lipid and nucleic acid, PEG-lipid, phospholipid, structural lipid). For instance, a lipid nanoparticle may comprise an amino lipid and a nucleic acid. Compositions comprising the lipid nanoparticles, such as those described herein, may be used for a wide variety of applications, including the stealth delivery of therapeutic payloads with minimal adverse innate immune response.

Effective in vivo delivery of nucleic acids represents a continuing medical challenge. Exogenous nucleic acids (i.e., originating from outside of a cell or organism) are readily degraded in the body, e.g., by the immune system. Accordingly, effective delivery of nucleic acids to cells often requires the use of a particulate carrier (e.g., lipid nanoparticles). The particulate carrier should be formulated to have minimal particle aggregation, be relatively stable prior to intracellular delivery, effectively deliver nucleic acids intracellularly, and illicit no or minimal immune response. To achieve minimal particle aggregation and pre-delivery stability, many conventional particulate carriers have relied on the presence and/or concentration of certain components (e.g., PEG-lipid). However, it has been discovered that certain components may decrease the stability of encapsulated nucleic acids (e.g., mRNA molecules). The reduced stability may limit the broad applicability of the particulate carriers. As such, there remains a need for methods by which to improve the stability of nucleic acid (e.g., mRNA) encapsulated within lipid nanoparticles.

In some embodiments, the lipid nanoparticles comprise one or more of ionizable molecules, polynucleotides, and optional components, such as structural lipids, sterols, neutral lipids, phospholipids and a molecule capable of reducing particle aggregation (e.g., polyethylene glycol (PEG), PEG-modified lipid), such as those described above.

In some embodiments, a LNP described herein may include one or more ionizable molecules (e.g., amino lipids or ionizable lipids). The ionizable molecule may comprise a charged group and may have a certain pKa. In certain embodiments, the pKa of the ionizable molecule may be greater than or equal to about 6, greater than or equal to about 6.2, greater than or equal to about 6.5, greater than or equal to about 6.8, greater than or equal to about 7, greater than or equal to about 7.2, greater than or equal to about 7.5, greater than or equal to about 7.8, greater than or equal to about 8. In some embodiments, the pKa of the ionizable molecule may be less than or equal to about 10, less than or equal to about 9.8, less than or equal to about 9.5, less than or equal to about 9.2, less than or equal to about 9.0, less than or equal to about 8.8, or less than or equal to about 8.5. Combinations of the above referenced ranges are also possible (e.g., greater than or equal to 6 and less than or equal to about 8.5). Other ranges are also possible. In embodiments in which more than one type of ionizable molecule are present in a particle, each type of ionizable molecule may independently have a pKa in one or more of the ranges described above.

In general, an ionizable molecule comprises one or more charged groups. In some embodiments, an ionizable molecule may be positively charged or negatively charged. For instance, an ionizable molecule may be positively charged. For example, an ionizable molecule may comprise an amine group. As used herein, the term “ionizable molecule” has its ordinary meaning in the art and may refer to a molecule or matrix comprising one or more charged moiety. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidazolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule and/or matrix may be selected as desired.

In some cases, an ionizable molecule (e.g., an amino lipid or ionizable lipid) may include one or more precursor moieties that can be converted to charged moieties. For instance, the ionizable molecule may include a neutral moiety that can be hydrolyzed to form a charged moiety, such as those described above. As a non-limiting specific example, the molecule or matrix may include an amide, which can be hydrolyzed to form an amine, respectively. Those of ordinary skill in the art will be able to determine whether a given chemical moiety carries a formal electronic charge (for example, by inspection, pH titration, ionic conductivity measurements, etc.), and/or whether a given chemical moiety can be reacted (e.g., hydrolyzed) to form a chemical moiety that carries a formal electronic charge.

The ionizable molecule (e.g., amino lipid or ionizable lipid) may have any suitable molecular weight. In certain embodiments, the molecular weight of an ionizable molecule is less than or equal to about 2,500 g/mol, less than or equal to about 2,000 g/mol, less than or equal to about 1,500 g/mol, less than or equal to about 1,250 g/mol, less than or equal to about 1,000 g/mol, less than or equal to about 900 g/mol, less than or equal to about 800 g/mol, less than or equal to about 700 g/mol, less than or equal to about 600 g/mol, less than or equal to about 500 g/mol, less than or equal to about 400 g/mol, less than or equal to about 300 g/mol, less than or equal to about 200 g/mol, or less than or equal to about 100 g/mol. In some instances, the molecular weight of an ionizable molecule is greater than or equal to about 100 g/mol, greater than or equal to about 200 g/mol, greater than or equal to about 300 g/mol, greater than or equal to about 400 g/mol, greater than or equal to about 500 g/mol, greater than or equal to about 600 g/mol, greater than or equal to about 700 g/mol, greater than or equal to about 1000 g/mol, greater than or equal to about 1,250 g/mol, greater than or equal to about 1,500 g/mol, greater than or equal to about 1,750 g/mol, greater than or equal to about 2,000 g/mol, or greater than or equal to about 2,250 g/mol. Combinations of the above ranges (e.g., at least about 200 g/mol and less than or equal to about 2,500 g/mol) are also possible. In embodiments in which more than one type of ionizable molecules are present in a particle, each type of ionizable molecule may independently have a molecular weight in one or more of the ranges described above.

In some embodiments, the percentage (e.g., by weight, or by mole) of a single type of ionizable molecule (e.g., amino lipid or ionizable lipid) and/or of all the ionizable molecules within a particle may be greater than or equal to about 15%, greater than or equal to about 16%, greater than or equal to about 17%, greater than or equal to about 18%, greater than or equal to about 19%, greater than or equal to about 20%, greater than or equal to about 210%, greater than or equal to about 22%, greater than or equal to about 23%, greater than or equal to about 24%, greater than or equal to about 25%, greater than or equal to about 30%, greater than or equal to about 35%, greater than or equal to about 40%, greater than or equal to about 42%, greater than or equal to about 45%, greater than or equal to about 48%, greater than or equal to about 50%, greater than or equal to about 52%, greater than or equal to about 55%, greater than or equal to about 58%, greater than or equal to about 60%, greater than or equal to about 62%, greater than or equal to about 65%, or greater than or equal to about 68%. In some instances, the percentage (e.g., by weight, or by mole) may be less than or equal to about 70%, less than or equal to about 68%, less than or equal to about 65%, less than or equal to about 62%, less than or equal to about 60%, less than or equal to about 58%, less than or equal to about 55%, less than or equal to about 52%, less than or equal to about 50%, or less than or equal to about 48%. Combinations of the above referenced ranges are also possible (e.g., greater than or equal to 20% and less than or equal to about 60%, greater than or equal to 40% and less than or equal to about 55%, etc.). In embodiments in which more than one type of ionizable molecule is present in a particle, each type of ionizable molecule may independently have a percentage (e.g., by weight, or by mole) in one or more of the ranges described above. The percentage (e.g., by weight, or by mole) may be determined by extracting the ionizable molecule(s) from the dried particles using, e.g., organic solvents, and measuring the quantity of the agent using high pressure liquid chromatography (i.e., HPLC), liquid chromatography-mass spectrometry (LC-MS), nuclear magnetic resonance (NMR), or mass spectrometry (MS). Those of ordinary skill in the art would be knowledgeable of techniques to determine the quantity of a component using the above-referenced techniques. For example, HPLC may be used to quantify the amount of a component, by, e.g., comparing the area under the curve of a HPLC chromatogram to a standard curve.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

As described herein, in some embodiments, mRNA is formulated with LNP, such that the mRNA is at least partially encompassed within the LNP. LNP-formulated mRNA vaccines (fLNPs) comprise a structure that protects the RNA from environmental components that may lead to mRNA degradation.

Insertions and Substitutions

The present disclosure also includes a polynucleotide of the present disclosure that further comprises insertions and/or substitutions.

In some embodiments, the 5′UTR of the polynucleotide can be replaced by the insertion of at least one region and/or string of nucleosides of the same base. The region and/or string of nucleotides can include, but is not limited to, at least 3, at least 4, at least 5, at least 6, at least 7 or at least 8 nucleotides and the nucleotides can be natural and/or unnatural. As a non-limiting example, the group of nucleotides can include 5-8 adenine, cytosine, thymine, a string of any of the other nucleotides disclosed herein and/or combinations thereof.

In some embodiments, the 5′UTR of the polynucleotide can be replaced by the insertion of at least two regions and/or strings of nucleotides of two different bases such as, but not limited to, adenine, cytosine, thymine, any of the other nucleotides disclosed herein and/or combinations thereof. For example, the 5′UTR can be replaced by inserting 5-8 adenine bases followed by the insertion of 5-8 cytosine bases. In another example, the 5′UTR can be replaced by inserting 5-8 cytosine bases followed by the insertion of 5-8 adenine bases.

In some embodiments, the polynucleotide can include at least one substitution and/or insertion downstream of the transcription start site that can be recognized by an RNA polymerase. As a non-limiting example, at least one substitution and/or insertion can occur downstream of the transcription start site by substituting at least one nucleic acid in the region just downstream of the transcription start site (such as, but not limited to, +1 to +6). Changes to region of nucleotides just downstream of the transcription start site can affect initiation rates, increase apparent nucleotide triphosphate (NTP) reaction constant values, and increase the dissociation of short transcripts from the transcription complex curing initial transcription (Brieba et al, Biochemistry (2002) 41: 5144-5149; herein incorporated by reference in its entirety). The modification, substitution and/or insertion of at least one nucleoside can cause a silent mutation of the sequence or can cause a mutation in the amino acid sequence.

In some embodiments, the polynucleotide can include the substitution of at least 1, at least 2, at least 3, at least 4, at least 5, at least 6, at least 7, at least 8, at least 9, at least 10, at least 11, at least 12 or at least 13 guanine bases downstream of the transcription start site.

In some embodiments, the polynucleotide can include the substitution of at least 1, at least 2, at least 3, at least 4, at least 5 or at least 6 guanine bases in the region just downstream of the transcription start site. As a non-limiting example, if the nucleotides in the region are GGGAGA, the guanine bases can be substituted by at least 1, at least 2, at least 3 or at least 4 adenine nucleotides. In another non-limiting example, if the nucleotides in the region are GGGAGA the guanine bases can be substituted by at least 1, at least 2, at least 3 or at least 4 cytosine bases. In another non-limiting example, if the nucleotides in the region are GGGAGA the guanine bases can be substituted by at least 1, at least 2, at least 3 or at least 4 thymine, and/or any of the nucleotides described herein.

In some embodiments, the polynucleotide can include at least one substitution and/or insertion upstream of the start codon. For the purpose of clarity, one of skill in the art would appreciate that the start codon is the first codon of the protein coding region whereas the transcription start site is the site where transcription begins. The polynucleotide can include, but is not limited to, at least 1, at least 2, at least 3, at least 4, at least 5, at least 6, at least 7 or at least 8 substitutions and/or insertions of nucleotide bases. The nucleotide bases can be inserted or substituted at 1, at least 1, at least 2, at least 3, at least 4 or at least 5 locations upstream of the start codon. The nucleotides inserted and/or substituted can be the same base (e.g., all A or all C or all T or all G), two different bases (e.g., A and C, A and T, or C and T), three different bases (e.g., A, C and T or A, C and T) or at least four different bases.

As a non-limiting example, the guanine base upstream of the coding region in the polynucleotide can be substituted with adenine, cytosine, thymine, or any of the nucleotides described herein. In another non-limiting example the substitution of guanine bases in the polynucleotide can be designed so as to leave one guanine base in the region downstream of the transcription start site and before the start codon (see Esvelt et al. Nature (2011) 472(7344):499-503; the contents of which is herein incorporated by reference in its entirety). As a non-limiting example, at least 5 nucleotides can be inserted at 1 location downstream of the transcription start site but upstream of the start codon and the at least 5 nucleotides can be the same base type.

In some embodiments, a polynucleotide includes 200 to 3,000 nucleotides. For example, a polynucleotide may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides.

Provided herein are compositions (e.g., pharmaceutical compositions), methods, kits and reagents for prevention and/or treatment of disease in humans and other mammals. The compositions can be used as therapeutic or prophylactic agents. For example, when the composition comprises a picornavirus capsid polyprotein and/or a picornavirus 3C protease the RNA encoding such a picornavirus capsid polyprotein and/or a picornavirus 3C protease is used to provide prophylactic or therapeutic protection from an Picornaviridae infection. Prophylactic protection from Picornaviridae infection can be achieved following administration of a composition (e.g., a composition comprising one or more polynucleotides encoding one or more picornavirus capsid polyprotein and/or a picornavirus 3C protease) of the present disclosure. In some embodiments, the Picornaviridae is a member of the genus Enterovirus. In some embodiments, the Enterovirus is any virus form the Enterovirus A-D species. In some embodiments, the Enterovirus is any virus from the Rhinovirus A-C species. Compositions can be administered once, twice, three times, four times or more. In some aspects, the compositions can be administered to an infected individual to achieve a therapeutic response. Dosing may need to be adjusted accordingly.

It is envisioned that there may be situations where persons are at risk for infection with more than one strain of type of infectious agent. RNA (mRNA) therapeutic treatments are particularly amenable to combination vaccination approaches due to a number of factors including, but not limited to, speed of manufacture, ability to rapidly tailor treatments to accommodate perceived geographical threat, and the like. To protect against more than one strain of Enterovirus, a combination treatment can be administered that includes RNA encoding at least one polypeptide (or portion thereof) of a picornavirus capsid polyprotein and further includes RNA encoding at least one polypeptide (or portion thereof) of a picornavirus 3C protease. RNAs (mRNAs) can be co-formulated, for example, in a single lipid nanoparticle (LNP) or can be formulated in separate LNPs destined for co-administration. In some embodiments, the Enterovirus is HRV16. In some embodiments, the Enterovirus is EV-D68. In some embodiments, the Enterovirus is EV-A71.

A prophylactically effective dose is a therapeutically effective dose that prevents infection with the virus at a clinically acceptable level. In some embodiments, the therapeutically effective dose is a dose listed in a package insert for the treatment. A prophylactic therapy as used herein refers to a therapy that prevents, to some extent, the infection from increasing. The infection may be prevented completely or partially.

The methods of the invention involve, in some aspects, passively immunizing a mammalian subject against an influenza virus infection. The method involves administering to the subject a composition comprising at least one RNA polynucleotide having an open reading frame encoding at least one picornavirus capsid polyprotein and picornavirus 3C protease. In some aspects, methods of the present disclosure provide prophylactic treatments against an Enterovirus infection. In some embodiments, the Enterovirus is HRV16. In some embodiments, the Enterovirus is EV-D68. In some embodiments, the Enterovirus is EV-A71.

Therapeutic methods of treatment are also included within the invention. Methods of treating an Enterovirus infection in a subject are provided in aspects of the disclosure. The method involves administering to the subject having an influenza virus infection a composition comprising at least one RNA polynucleotide having an open reading frame encoding at least one picornavirus capsid polyprotein and picornavirus 3C protease. In some embodiments, the Enterovirus is HRV16. In some embodiments, the Enterovirus is EV-D68. In some embodiments, the Enterovirus is EV-A71.

As used herein, the terms treat, treated, or treating when used with respect to a disorder such as a viral infection, refers to a treatment which increases the resistance of a subject to development of the disease or, in other words, decreases the likelihood that the subject will develop the disease in response to infection with the virus as well as a treatment after the subject has developed the disease in order to fight the infection or prevent the infection from becoming worse.

An “effective amount” of an RNA treatment of the present disclosure is provided based, at least in part, on the target tissue, target cell type, means of administration, physical characteristics of the polynucleotide (e.g., size, and extent of modified nucleosides), and other components of the RNA treatment, and other determinants. Increased antibody production may be demonstrated by increased cell transfection (the percentage of cells transfected with the RNA treatment), increased protein translation from the polynucleotide, decreased nucleic acid degradation (as demonstrated, for example, by increased duration of protein translation from a modified polynucleotide), or altered response of the host cell.

In some embodiments, RNA treatments (including polynucleotides and their encoded polypeptides) in accordance with the present disclosure may be used for treatment of the disease.

RNA treatments may be administered prophylactically or therapeutically as part of an active immunization scheme to healthy individuals or early in infection during the incubation phase or during active infection after onset of symptoms. In some embodiments, the amount of RNA treatments of the present disclosure provided to a cell, a tissue or a subject may be an amount effective for immune prophylaxis.

RNA treatments may be administered with other prophylactic or therapeutic compounds. As a non-limiting example, a prophylactic or therapeutic compound may be a vaccine containing an virus treatment with or without an adjuvant or a booster. As used herein, when referring to a prophylactic composition, such as a treatment or vaccine, the term “booster” refers to an extra administration of the prophylactic composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years, or more than 99 years. In exemplary embodiments, the time of administration between the initial administration of the prophylactic composition and the booster may be, but is not limited to, 1 week, 2 weeks, 3 weeks, 1 month, 2 months, 3 months, 6 months, or 1 year.

In some embodiments, RNA treatments may be administered subcutaneously, intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs.

Provided herein are pharmaceutical compositions including RNA treatments and RNA compositions and/or complexes optionally in combination with one or more pharmaceutically acceptable excipients.

RNA treatments may be formulated or administered in combination with one or more pharmaceutically-acceptable excipients. In some embodiments, compositions comprise at least one additional active substance, such as, for example, a therapeutically-active substance, a prophylactically-active substance, or a combination of both. Treatment compositions may be sterile, pyrogen-free or both sterile and pyrogen-free. General considerations in the formulation and/or manufacture of pharmaceutical agents, such as treatment compositions, may be found, for example, in Remington: The Science and Practice of Pharmacy 21st ed., Lippincott Williams & Wilkins, 2005 (incorporated herein by reference in its entirety).

In some embodiments, RNA treatments are administered to humans, human patients, or subjects. For the purposes of the present disclosure, the phrase “active ingredient” generally refers to the RNA treatments or the polynucleotides contained therein, for example, RNA polynucleotides (e.g., mRNA polynucleotides) encoding picornavirus capsid polyprotein and/or picornavirus 3C protease.

Formulations of the compositions described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

RNA treatments can be formulated using one or more excipients to: (1) increase stability: (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (e.g., HCAb) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with RNA treatments (e.g., for transplantation into a subject), hyaluronidase, nanoparticle mimics, and combinations thereof.

Naturally-occurring eukaryotic mRNA molecules have been found to contain stabilizing elements, including, but not limited to untranslated regions (UTR) at their 5′-end (5′UTR) and/or at their 3′-end (3′UTR), in addition to other structural features, such as a 5′-cap structure or a 3′-poly(A) tail. Both the 5′UTR and the 3′UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing. The 3′-poly(A) tail is typically a stretch of adenine nucleotides added to the 3′-end of the transcribed mRNA. It can comprise up to about 400 adenine nucleotides. In some embodiments the length of the 3′-poly(A) tail may be an essential element with respect to the stability of the individual mRNA.

In some embodiments, the RNA treatment may include one or more stabilizing elements. Stabilizing elements may include, for instance, a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it is peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, the RNA treatments include a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine:guanine phosphoribosyl transferase (GPT)).

In some embodiments, the combination of a poly(A) sequence or polyadenylation signal and at least one histone stem-loop, even though both represent alternative mechanisms in nature, acts synergistically to increase the protein expression beyond the level observed with either of the individual elements. It has been found that the synergistic effect of the combination of poly(A) and at least one histone stem-loop does not depend on the order of the elements or the length of the poly(A) sequence.

In some embodiments, the polynucleotides described herein can be formulated in lipid nanoparticles having a diameter from about 1 nm to about 100 nm such as, but not limited to, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles can have a diameter from about 10 to 500 nm. In one embodiment, the lipid nanoparticle can have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the polynucleotides can be delivered using smaller LNPs. Such particles can comprise a diameter from below 0.1 μm up to 100 nm such as, but not limited to, less than 0.1 μm, less than 1.0 μm, less than 5 μm, less than 10 μm, less than 15 um, less than 20 um, less than 25 um, less than 30 um, less than 35 um, less than 40 um, less than 50 um, less than 55 um, less than 60 um, less than 65 um, less than 70 um, less than 75 um, less than 80 um, less than 85 um, less than 90 um, less than 95 um, less than 100 um, less than 125 um, less than 150 um, less than 175 um, less than 200 um, less than 225 um, less than 250 um, less than 275 um, less than 300 um, less than 325 um, less than 350 um, less than 375 um, less than 400 um, less than 425 um, less than 450 um, less than 475 um, less than 500 um, less than 525 um, less than 550 um, less than 575 um, less than 600 um, less than 625 um, less than 650 um, less than 675 um, less than 700 um, less than 725 um, less than 750 um, less than 775 um, less than 800 um, less than 825 um, less than 850 um, less than 875 um, less than 900 um, less than 925 um, less than 950 um, or less than 975 um.

The nanoparticles and microparticles described herein can be geometrically engineered to modulate macrophage and/or the immune response. The geometrically engineered particles can have varied shapes, sizes and/or surface charges to incorporate the polynucleotides described herein for targeted delivery such as, but not limited to, pulmonary delivery (see, e.g., Intl. Pub. No. WO2013082111, herein incorporated by reference in its entirety). Other physical features the geometrically engineering particles can include, but are not limited to, fenestrations, angled arms, asymmetry and surface roughness, charge that can alter the interactions with cells and tissues.

RNA treatments may be administered by any route which results in a therapeutically effective outcome. These include, but are not limited, to intradermal, intramuscular, and/or subcutaneous administration. The present disclosure provides methods comprising administering RNA treatments to a subject in need thereof. The exact amount required will vary from subject to subject, depending on the species, age, and general condition of the subject, the severity of the disease, the particular composition, its mode of administration, its mode of activity, and the like. RNA treatments compositions are typically formulated in dosage unit form for ease of administration and uniformity of dosage. It will be understood, however, that the total daily usage of RNA treatments compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, RNA treatments compositions may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see e.g., the range of unit doses described in International Publication No WO2013078199, herein incorporated by reference in its entirety). The desired dosage may be delivered three times a day, two times a day, once a day, every other day, every third day, every week, every two weeks, every three weeks, every four weeks, every 2 months, every three months, every 6 months, etc. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In exemplary embodiments, RNA treatments compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg.

In some embodiments, RNA treatment compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, RNA treatment compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.0100 mg, 0.025 mg, 0.050 mg, 0.075 mg, 0.100 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, an RNA treatment composition may be administered three or four times.

In some embodiments, RNA treatment compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.010 mg, 0.025 mg, 0.100 mg or 0.400 mg.

In some embodiments, the RNA for use in a method of treating a subject is administered to the subject in a single dosage of between 10 μg/kg and 400 μg/kg of the nucleic acid treatment in an effective amount to treat the subject. In some embodiments, the RNA treatment for use in a method of treating a subject is administered to the subject in a single dosage of between 10 μg and 400 μg of the nucleic acid treatment in an effective amount to treat the subject.

An RNA pharmaceutical composition described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

This disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EXAMPLES

### Example 1: P1+3CD mRNAs Demonstrates Proper P1 Processing In Vitro

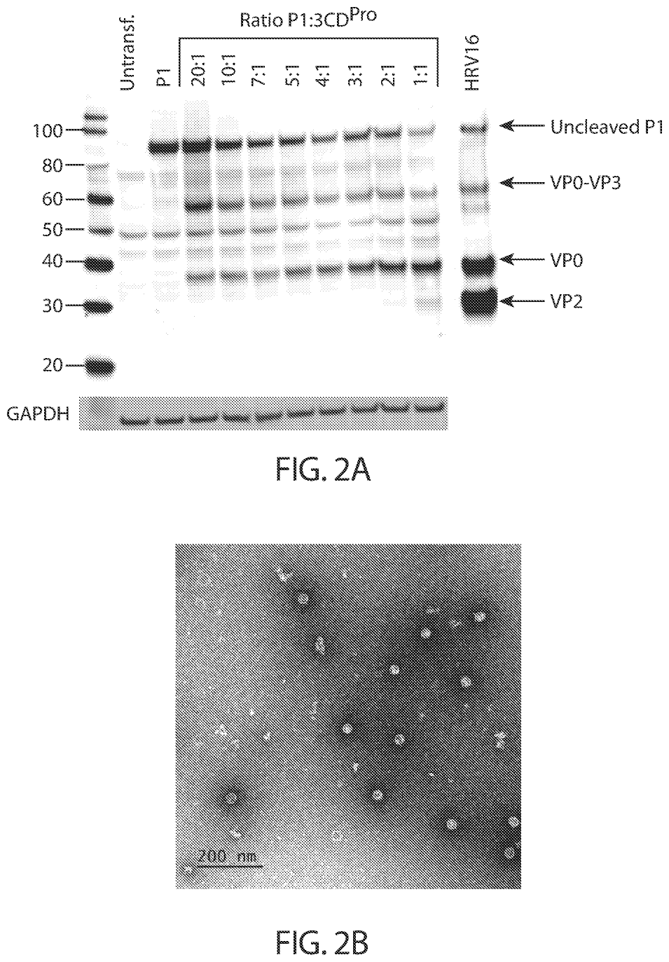

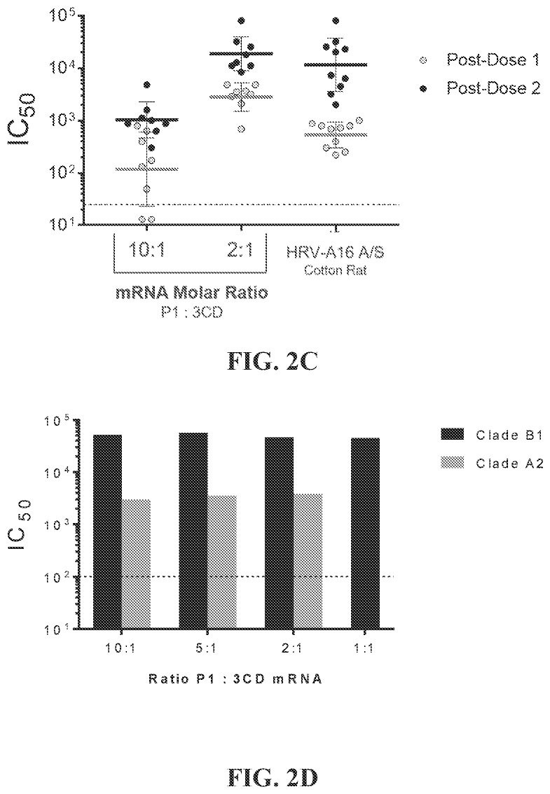

To investigate whether mRNAs encoding P1 and 3CD would be sufficient produce a virus-like particle (VLP), an in vitro assay was conducted as follows. Two mRNAs, one encoding human rhinovirus (HRV)-A16 P1 and the other encoding HRV-A16 3CD protease, were co-transfected into cells to determine i) whether P1 was expressed; ii) whether 3CD was expressed and able to catalyze cleavage of P1 into constituent VP proteins intracellularly; and ii) whether there is an optimal ratio of P1:3CD mRNA that promotes more efficient cleavage of P1 while minimizing 3CD-driven cytotoxicity. In FIG. 2A, the amount of P1 mRNA transfected was held constant at 2 μg, while increasing amounts of 3CD mRNA were co-transfected to give the indicated molar ratios of P1:3CD. In a Western blot, cell lysates were blotted with an anti-HRV-A16 VP2 antibody, which also recognizes VP0 and uncleaved P1. Transfection of 3CD mRNA at any of the tested amounts resulted in intracellular cleavage of P1. Increasing amounts of 3CD led to more distinct loss of the high molecular weight band representing uncleaved P1 and a greater increase in the amount of lower molecular weight bands of individual proteins (VP2) and smaller polyproteins (VP0). These bands corresponded in size to bands seen in lysate prepared from purified wild-type HRV-A16 virions and likely represent, from largest to smallest, VP0-VP3 precursor, VP0 and VP2 (FIG. 2A).

Transfection of any amount of the polyprotein encoding the proteolytic 3C in the form of 3CD mRNA led to demonstrable cleavage of P1, but increasing amounts led to greater “conversion” of P1 into VP0 and, at the highest amounts, appearance of a band corresponding in size to VP2. However, larger amounts of 3CD mRNA concomitantly increased cytotoxicity in a dose-dependent manner, with the 1:1 ratio of P1:3CD demonstrating the most cytotoxicity. The polyprotein 3CD was used instead of 3C to avoid possible excessive cellular cytotoxicity of 3C.

### Example 2: Virus-Like Particles are Produced by Co-Expression of P1+3CD

To further evaluate whether co-expression of P1 and 3CD would be sufficient to produce a VLP, a large-scale transfection and purification experiment was carried out using two DNA plasmids that encoded HRV-A16 P1 and HRV-A16 3CD respectively. Plasmids were transfected at a mass ratio of 4 parts P1 to 1 part 3CD, with the goal of potentially increasing VLP yield by slowing down cell death resulting from 3CD-driven cytotoxicity. VLPs were purified from the supernatant of transfected cells using PEG precipitation followed by size exclusion chromatography. Negative staining transmission electron microscopy revealed uniform particles of the size expected for rhinovirus virions (˜30 nanometers in diameter) (FIG. 2B). These data demonstrate that P1 and 3CD co-expression leads to both proper processing of P1 and assembly of individual VP proteins into virus-like particles that are released into the extracellular space and resemble wild-type virions morphologically.

### Example 3: Vaccine Comprising P1+3CD mRNAs Generates Neutralizing Antibody Titers in Mice

To investigate whether administration of mRNAs encoding two polyproteins P1+3CD mRNAs could result in formation of VLPs that were immunogenic and could result in production of HRVA-16 neutralizing antibodies, an in vivo assay was conducted as follows. Mice received a prime-boost vaccine regimen consisting of 10 μg P1 mRNA and, due to concerns about toxicity, two different amounts of 3CD mRNA, resulting in final P1:3CD molar ratios of 10:1 and 2:1. FIG. 2C shows the HRV-A16 neutralizing antibody titers three weeks after the first vaccination (Post-Dose 1) and two weeks after the boost (Post-Dose 2). Both the 10:1 and 2:1 ratios resulted in detectable neutralizing titers, but a single dose of the 2:1 ratio resulted in higher titers than two doses of the 10:1 ratio. Serum from cotton rats injected 2× with live HRV-A16 was included as a benchmark control. Surprisingly, two injections of the P1:3CD 2:1 ratio combination resulted in higher neutralizing titers in mice than two doses of replicative HRV-A16 administered IM to cotton rats. No adverse events were observed in mice following administration of either the 10:1 or 2:1 ratios, suggesting that the cytotoxicity seen in vivo following transfection of 3CD mRNA is not necessarily predictive of effects in vivo.

### Example 4: P1+3CD mRNAs Prove Immunogenic for Other Species within the Enterovirus Genus

Following the approach used for HRV-A16 in Example 2, mRNAs encoding two polyproteins, where one has enzymatic activity for the other protein, P1 and 3CD were generated from Enterovirus D68 (EV-D68) isolate US/MO/14-18950. This isolate was collected from a human patient in Missouri during the 2014 EV-D68 outbreak in the United States and is a Clade B1 isolate. For this study, mice were vaccinated with an expanded set of P1:3D ratios. The amount of P1 mRNA was kept constant at 10 μg, while increasing amounts of 3CD mRNA were added to give final P1:3CD molar ratios of 10:1, 5:1, 2:1 and 1:1. Vaccination followed the same two-dose schedule used for HRV-A16. Serum was collected two weeks post-boost, pooled for each group, and then tested for neutralization of a different Clade B1 isolate (US/MO/14-18947) and a Clade A2 isolate (US/KY/14-18953). For each isolate, neutralization titers were similar across all four ratios tested (FIG. 2D). Neutralization titers were approximately one log higher for Clade B1 isolate MO/14-18947. This was expected, as the sequence of the P1 mRNA is also derived from a Clade B1 isolate from Missouri 2014 and thus shares a high degree of sequence homology. However, robust neutralizing titers were also generated against the more divergent Clade A2 isolate. This suggests that, in the case of EV-D68, vaccination with a single P1 mRNA will generate neutralizing antibodies that are effective across clades. This result also shows that the protease can function when the P1 substrate is from a different viral isolate and species.

The combined data from these two studies suggests that administration of polyproteins P1+3CD mRNA in LNPs, at a molar ratio of 2 parts P1 to 1 part 3CD, is highly immunogenic and effective at inducing neutralizing antibodies to the resulting VLPs.

To further investigate the efficacy of this P1+3CD combination when delivered at lower doses (while maintaining the 2:1 molar ratio), a dose range study was conducted across an expanded group of Enterovirus species. Table 1 lists the targeted Picornavirus species and genotypes, the isolate used to design the sequences of the P1 and 3CD mRNAs, the doses of total mRNA (P1+3CD) administered to mice, and the respective amounts of P1 mRNA within the dose. For this dose range study, mRNA was diluted in a 5-fold dilution series, and at least three doses were tested.

TABLE 1
Expanded group of Enterovirus species used in dose range study

| | | Isolate used for P1/3CD | | μg P1 mRNA in |
|---|---|---|---|---|
| Species | Genotype | mRNA sequence | Dose (μg total mRNA) | dose |
| Rhinovirus A | HRV-A16 | HRV-A16 | 6.88, 1.38, 0.28, 0.055 | 5, 1, 0.2, 0.04 |
| Rhinovirus B | HRV-B14 | HRV-B14 | 6.88, 1.38, 0.28 | 5, 1, 0.2 |
| Enterovirus D | EV-D68 | US/MO/14-18950 | 6.88, 1.38, 0.28 | 5, 1, 0.2 |
| Enterovirus A | EV-A71 | AH08/06 Subgenotype C4a | 6.88, 1.38, 0.28 | 5, 1, 0.2 |

The vaccination schedule was identical to the previous studies. Mice were vaccinated on Days 1 and 22, and serum was collected three weeks post-dose 1 (day 21) and two weeks post-dose 2 (day 35). The post-dose 2 serum was tested for neutralizing antibody titers in the corresponding neutralization assays. For HRV-A16 and HRV-B14, the viruses used in the neutralization assays were identical to the viral sequences used in the respective P1 and 3CD mRNAs. For EV-D68 and EV-A71, the serum from vaccinated mice was tested for neutralization across two or more heterologous isolates that represented different clades.

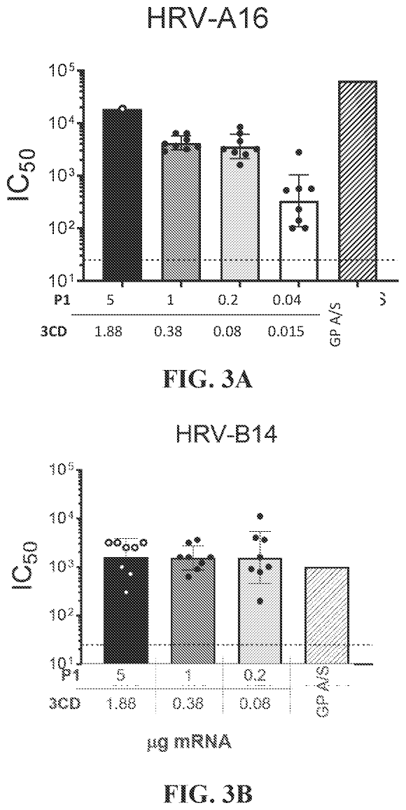

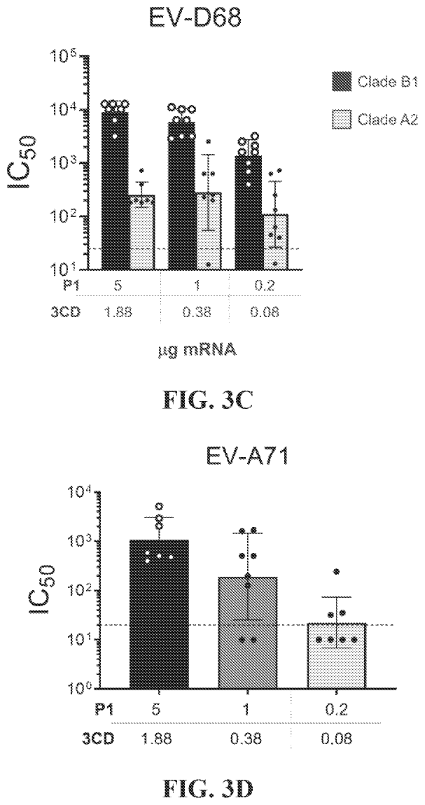

FIGS. 3A-3D show the neutralizing antibody titers two weeks after the second vaccination (Day 35). FIG. 3A shows the neutralizing antibody titers for animals vaccinated with HRV-A16 P1:3CD. Animals received doses ranging from 5 μg to 0.04 μg HRV-A16 P1 mRNA, plus the corresponding amount of HRV-A16 3CD mRNA to maintain the 2:1 ratio of P1 and 3CD. Commercial HRV-A16 antiserum from hyperimmune guinea pigs was included as a benchmark control. All animals at all doses tested generated neutralizing antibody titers above the lower limit of detection of the assay, and titers exhibited dose dependence. Although none of the doses led to titers equal to or exceeding that of the guinea pig hyperimmune antiserum, the 5 μg dose resulted in titers that were higher than those generated in cotton rats receiving two doses of replicative HRV-A16 (FIG. 2C).

FIG. 3B shows the neutralizing antibody titers for animals vaccinated with HRV-B14 P1:3CD. Animals received doses ranging from 5 μg to 0.2 μg HRV-B14 P1 mRNA and the corresponding amount of HRV-B14 3CD mRNA to maintain the 2:1 ratio. Commercial HRV-B14 antiserum from hyperimmune guinea pigs was included as a benchmark control. Surprisingly, the three doses tested here for HRV-B14 all resulted in similar levels of neutralizing antibodies, and the geometric mean titer (GMT) for all doses exceeded the titer for the guinea pig hyperimmune HRV-B14 antiserum.

In FIG. 3C, animals were vaccinated with EV-D68 P1+3C) mRNA, with doses ranging from 5 μg to 0.2 μg P1 mRNA. Serum from these animals was tested for neutralization of heterologous EV-D68 isolates from Clades B1 and A2.

In FIG. 3D, animals were vaccinated with EV-A71 P1+3CD mRNA, with doses ranging from 5 μg to 0.2 μg P1 mRNA. Serum from these animals was tested for neutralization of heterologous genotype of EV-A71. Virus USA/2018.23092 is a clinical isolate recovered from the stool of a patient suffering from acute flaccid myelitis (AFM).

An effective human rhinovirus vaccine should provide protection against multiple serotypes within each of the three rhinovirus species: Rhinovirus A, B and C. Given P1's high intra-species sequence diversity, it is likely that adequate coverage of even a single species will require many different P1 mRNAs. However, in contrast to P1, the amino acid sequence of the 3CD protease is well-conserved among serotypes within a species, and conserved to a lesser degree across the three species. Alignments depicting consensus amino acid sequences of the 3C region of the 3CD protease for 79 serotypes of the Rhinovirus A species and 26 serotypes of the Rhinovirus B species show that residues within 4A of the P1 binding sites are highly conserved among HRV-A and HRV-B and that five sites differ between HRV-A and HRV-B while two of these sites differ within HRV-B. These putative binding residues are highly conserved within, and in some cases across, Rhinovirus A and Rhinovirus B. Such alignments further show that the VP2-VP3 cleavage site (Q-G-L-P) and VP3 N-terminus is highly conserved among both HRV-A and HRV-B. In contrast, the cleavage site and termini of VP3/VP1 differs between HRV-A and HRV-B, but is conserved within groups, with the conserved amino acid residues of Q/N-P-[IV]-E in HRV-A and A-L-[EMPT]-E/G-[FL] in HRV-B, respectively. The 3CD protease binds P1 and cleaves it in two locations: between VP2-VP3, and between VP3-VP1. When the P1 amino acid sequences at and around those cleavage sites are compared, there is evidence of sequence conservation. The VP3-VP1 cleavage site region exhibits conservation among serotypes within the species, while the VP2-VP3 cleavage site has a high degree of sequence homology across Rhinovirus A and Rhinovirus B species.

### Example 5: A Combination of [P1_{B14}+3CD_{A16}] Demonstrated Similar P1 Processing In Vitro and Immunogenicity In Vivo as the “Matched” Combination of [P1+3CD]_{B14}

The sequence conservation, in both 3CD protease's P1 binding sites, and in the cleavage site regions of P1 itself, suggests that a “matching” 3CD mRNA may not need to be provided for each P1 mRNA. It also suggests that a single 3CD protease may be able to process P1 proteins into VLPs from many serotypes within a species, or even across serotypes from two different species. To investigate whether the combination of [P1_{B14}+3CD_{A16}] demonstrated similar P1 processing in vitro as the previously-used “matched” combination of [P1+3CD]_{B14}, an in vitro assay was conducted as follows. P1_{A16} and P1_{B14} mRNA was transfected into cells, either alone or in combination with the indicated 3CD mRNA at a molar ratio of 2:1. Cell lysates were blotted with a pan-rhinovirus anti-VP1 antibody. Arrows indicate uncleaved P1 precursor protein and a lower molecular weight band corresponding to VP1 (FIG. 4A). For P1_{B14}, addition of either 3CD_{A16} or 3CD_{B14} mRNA leads to disappearance of the P1 band and appearance of VP1. The intensity of the VP1 band indicates that P1 cleavage is likely more efficient using the “matched” 3CD_{B14} mRNA, but the heterologous 3CD_{A16} protease is still able to process P1_{B14} into the constituent VP proteins.

To investigate whether the combination of [P1_{B14}+3CD_{A16}] demonstrated similar immunogenicity in vivo as the previously-used “matched” combination of [P1+3CD]_{B14}, an in vivo assay was conducted as follows. Mice were vaccinated with either [P1+3CD]_{A16} mRNA, or the mixed combination of [P1_{B14}+3CD_{A16}], at 2:1 molar ratio of P1:3CD mRNA (2.5 μg P1+0.94 μg 3CD). Because VLPs consist solely of VP proteins derived from the processing and subsequent maturation of the P1 precursor protein, the serotype specificity of the neutralizing antibody response is determined only by the P1 sequence, not the 3CD sequence. Therefore, the matched [P1+3CD]_{A16} combination was tested for neutralization of HRV-A16, while the mixed [P1_{B14}+3CD_{A16}] combination was tested for neutralization of HRV-B14. FIG. 4B shows the neutralizing antibody titers for the respective combinations, with commercial hyperimmune antiserum for each virus included as a benchmark control. As expected, vaccination with [P1+3CD]_{A16} resulted in a high HRV-A16 neutralizing antibody titer. The GMT for this group (2.5 μg P1; GMT=13024) corresponds with the GMTs of the 5 μg P1 and 1 μg P1 dose groups of the HRV-A16 dose range study in FIG. 2A (GMT=18549, GMT=4198, respectively). Vaccination with the mixed [P1_{B14}+3CD_{A16}] combination also produced HRV-B14 neutralizing antibodies, but not to the extent seen with matched [P1+3CD]_{B14}. The GMT for the mixed group, which received 2.5 μg HRV-B14 P1 mRNA, was 253. In contrast, the GMTs for groups receiving 5, 1, and 0.2 μg HRV-B14 P1 mRNA were 1617, 1554 and 1582, respectively (FIG. 2B). This indicates that the “matched” [P1+3CD]_{B14} combination is more effective than the mixed [P1_{B14}+3CD_{A16}] combination, even at less than 1/10^{th} the dose. This deficit might be due to the decrease in P1_{B14} processing seen in FIG. 4A, or to some unknown downstream effect, such as reduced assembly of VLPs. However, it is encouraging that production of HRV-B14 neutralizing antibodies was not completely abrogated with the use of HRV-A16 3CD. If 3CD from a Rhinovirus A serotype can function, even to a reduced extent, with P1 from a Rhinovirus B serotype, it is highly likely that coverage of all the serotypes within a species will require minimal 3CD sequences or perhaps careful selection of the CD sequence to be administered. Given the large amount of P1 sequences that would likely need to be included in a rhinovirus vaccine, it would be extremely useful to be able to include a few representative or consensus 3CD sequences, rather than an individual 3CD mRNA “matched” to each P1 mRNA.

### Example 6: HRV-A16 P1+3CD mRNA Vaccination Provides Protection from Infection in the Cotton Rat HRV Challenge Model

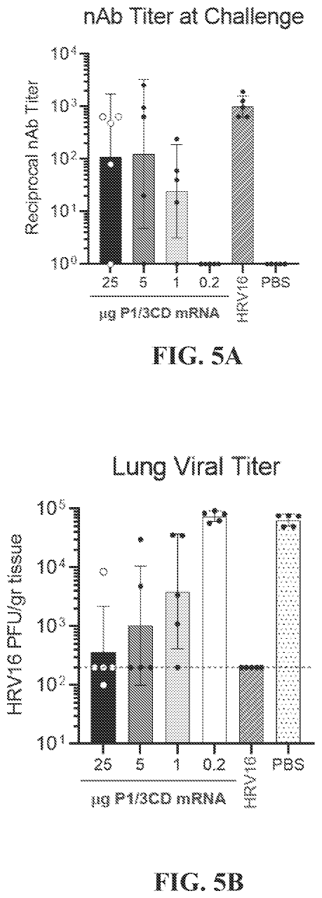

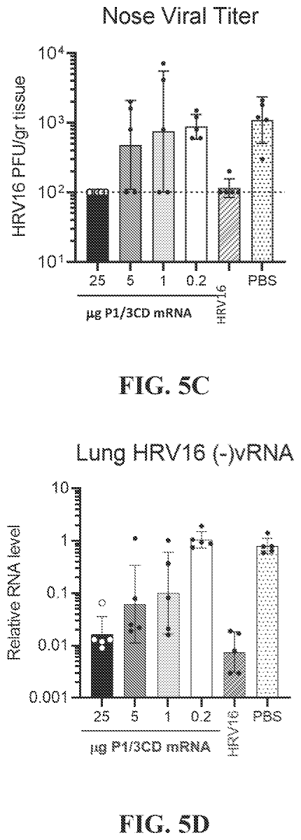

In order to investigate whether the P1+3CD mRNA combination can provide protection from infection, the efficacy of HRV-A16 P1+3CD mRNA vaccination was tested in a cotton rat HRV challenge model as follows. Animals were immunized IM on Day 1 and Day 22 with decreasing amounts of the HRV-A16 P1/3CD mRNA-LNP drug product. As a positive control, one group of animals received two IM injections of live replicative HRV-A16 virus, following the same schedule. The negative control group received PBS. Animals were challenged with live HRV-A16 virus administered intranasally on Day 35. At eight hours post-challenge, lung and nose were harvested and homogenized to calculate viral load. Serum was collected immediately prior to challenge to measure circulating neutralizing antibody levels. Vaccination with mRNAs encoding P1/3CD LNPs led to a dose-dependent induction of neutralizing antibodies (FIG. 5A), with some animals in the 25 μg and 5 μg groups having titers that were close to, or exceeded, those of animals receiving live HRV-A16. For each of the 3 higher doses of P1/3CD, there was one animal in each group that had no detectable titer, and none of the animals in the lowest dose group (0.2 μg) had detectable titers. Animals also exhibited a dose-dependent decrease in lung and nose viral loads. For the 25 μg and 5 μg groups, 4/5 and 3/5 animals, respectively, showed complete protection of lung tissue (FIG. 5B). All animals in the 25 μg dose group, and 2/5 animals in the 5 μg and 1 μg groups, showed complete protection of the nose tissue (FIG. SC). Animals in the 0.2 μg dose group exhibited no protection, and had lung and nose viral loads comparable to the PBS negative control group. In addition, a strong reduction of levels of lung viral RNA was detected in all animals receiving 25 μg, and 4/5 and 3/5 animals receiving 5 μg or 1 μg, respectively (FIG. 5D).

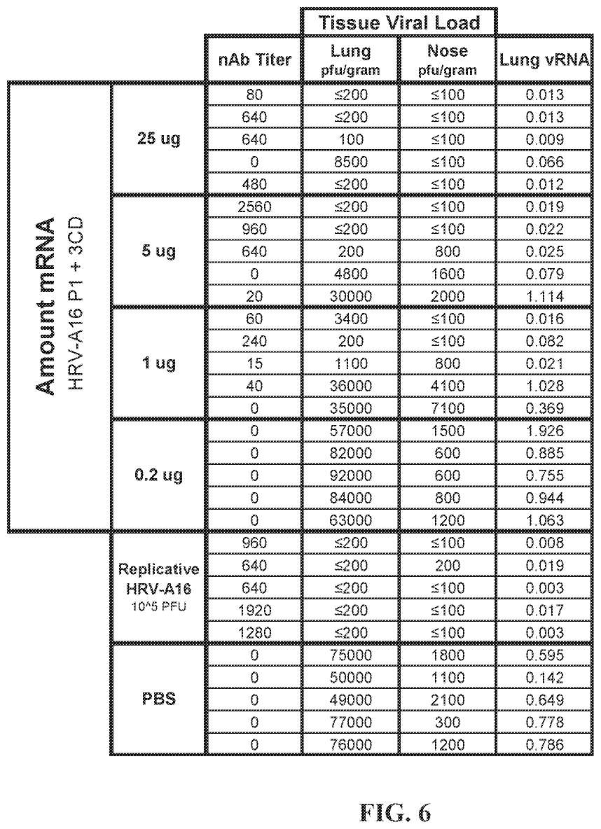

FIG. 6 includes the data generated for each animal for each endpoint assay. These results demonstrate that vaccination with HRV-A16 P1+3CD mRNA leads to dose-dependent protection from HRV-A16 infection, and that the level of protection achieved with the 25 μg dose is nearly identical to that generated by two injections of replicative virus.

TABLE 2
Sequences

SEQ
ID
NO:

HRV-A16 P1

| Chemistry: 1-methylpseudouridine | | |
|---|---|---|
| Cap: 7mG(5′)ppp(5′)NlmpNp | | |
| SEQ ID NO: 1 consists of from 5′ end to 3′ end: 5′ UTR SEQ ID NO: 2, | | 1 |
| mRNA ORF SEQ ID NO: 3, and 3′ UTR SEQ ID NO: 4. | | |
| 5′ UTR | GGGAAATAAGAGAGAAAAGAAGAGTAAGAAGAAATATAAGACCCCGGCG | 2 |
| | CCGCCACC | |
| ORF of | ATGGGCGCCCAGGTGAGCAGACAGAACGTGGGCACCCACAGCACCCAGAA | 3 |
| mRNA | CAGCGTGAGCAACGGCAGCAGCCTGAACTACTTCAACATCAATTACTTCAA | |
| Construct | GGACGCCGCCTCATCAGGCGCCAGCCGGCTGGACTTCAGCCAGGACCCCA | |
| (excluding | GCAAGTTCACCGACCCCGTGAAGGACGTGCTGGAGAAGGGCATCCCCACC | |
| stop codon) | CTGCAGAGCCCTAGCGTGGAGGCCTGTGGCTACAGCGACCGGATCATCCA | |
| | GATCACCCGGGGCGACTCAACCATCACCAGCCAGGACGTGGCCAACGCCG | |
| | TGGTGGGCTACGGCGTGTGGCCCCATTACCTGACCCCACAGGACGCCACCG | |
| | CCATCGACAAGCCCACCCAGCCTGACACCAGCAGCAACCGGTTCTACACC | |
| | CTGGACAGCAAGATGTGGAACAGCACCAGCAAGGGCTGGTGGTGGAAGCT | |
| | GCCCGACGCTCTGAAGGACATGGGCATCTTCGGCGAGAATATGTTCTACCA | |
| | CTTTCTGGGGAGAAGCGGCTACACCGTGCACGTGCAGTGCAACGCCAGCA | |
| | AGTTCCACCAGGGCACCCTGCTTGTGGTGATGATCCCCGAGCACCAGCTGG | |
| | CCACCGTGAACAAGGGCAACGTGAACGCCGGCTACAAGTACACCCATCCT | |
| | GGGGAGGCAGGCAGAGAGGTGGGCACCCAGGTGGAGAACGAGAAGCAGC | |
| | CCAGCGACGACAACTGGCTGAACTTCGACGGCACTCTGCTGGGCAACCTG | |
| | CTGATCTTTCCCCACCAGTTCATCAACCTGCGGAGCAACAATAGCGCCACC | |
| | CTGATCGTGCCCTACGTCAACGCCGTACCCATGGACAGCATGGTGCGGCAC | |
| | AACAACTGGAGCCTGGTGATCATCCCCGTGTGCCAGCTGCAGAGCAACAA | |
| | CATCAGCAACATCGTGCCCATCACCGTGAGCATCAGCCCCATGTGCGCCGA | |
| | GTTCAGCGGTGCTAGAGCCAAGACCGTGGTGCAGGGTCTGCCAGTGTACG | |
| | TGACACCAGGCAGCGGCCAGTTCATGACCACCGACGACATGCAGAGCCCT | |
| | TGCGCACTGCCCTGGTACCACCCCACCAAAGAGATCTTCATCCCCGGCGAG | |
| | GTGAAGAACCTGATCGAGATGTGCCAGGTGGACACCCTGATCCCCATCAA | |
| | CAGCACCCAGAGCAACATCGGCAACGTGAGCATGTACACCGTGACCCTGA | |
| | GCCCTCAGACCAAGCTGGCCGAGGAGATCTTTGCCATCAAGGTGGACATC | |
| | GCCAGCCATCCCCTGGCTACCACTCTGATCGGCGAGATCGCCAGCTACTTC | |
| | ACCCACTGGACCGGTAGCCTGCGGTTCAGCTTCATGTTCTGCGGCACCGCC | |
| | AACACCACCCTGAAGGTGCTGCTGGCCTACACGCCTCCTGGCATCGGGAA | |
| | GCCCAGGAGCAGGAAGGAGGCCATGCTGGGCACCCACGTGGTGTGGGACG | |
| | TGGGCCTGCAGAGCACCGTGAGCCTGGTGGTGCCCTGGATCAGCGCCAGC | |
| | CAGTACCGGTTCACCACTCCCGACACCTACAGCAGCGCCGGCTACATCACC | |
| | TGCTGGTACCAGACCAACTTCGTGGTGCCACCCAACACACCAAACACCGCC | |
| | GAGATGCTGTGCTTCGTGAGCGGCTGCAACCACTTCTGCCTGCGGATGGCC | |
| | AGAGACACCGACCTGCACAAGCAGACCGGCCCCATCACCCAGAACCCCGT | |
| | GGAGAACTACATCGACGAGGTGCTGAACGAGGTCCTGGTGGTGCCCAATA | |
| | TCAACCAGAGCCACCCCACCACCTCTAACGCCGCTCCTGTGCTGGACGCTG | |
| | CCGAAACCGGACACACCAACAAGATCCAGCCCGAGGACACCATCGAGACC | |
| | CGGTACGTGCAGAGCAGCCAGACCCTGGACGAGATGAGCGTGGAGAGCTT | |
| | TCTGGGTAGGAGCGGCTGCATCCACGAGAGCGTGCTGGACATCGTGGACA | |
| | ACTACAACGACCAGAGCTTCACCAAGTGGAACATCAACCTGCAGGAGATG | |
| | GCCCAGATCAGGCGGAAGTTCGAGATGTTCACCTACGCCCGGTTCGACAG | |
| | CGAGATCACCATGGTGCCCAGCGTTGCCGCTAAGGACGGCCACATTGGCC | |
| | ACATCGTGATGCAGTACATGTACGTTCCTCCAGGCGCCCCTATCCCTACCA | |
| | CCCGGGACGACTACGCTTGGCAGAGCGGCACCAACGCCAGCGTGTTCTGG | |
| | CAGCACGGACAGCCCTTCCCTCGGTTCAGCCTGCCCTTCCTGAGCATCGCC | |
| | AGCGCCTACTACATGTTCTACGACGGATACGACGGCGACACCTACAAGAG | |
| | CCGGTACGGCACCGTGGTGACCAACGACATGGGCACCCTGTGCAGCCGGA | |
| | TCGTGACCAGCGAGCAGCTGCACAAGGTGAAGGTGGTGACCCGGATCTAC | |
| | CACAAGGCCAAGCACACCAAGGCCTGGTGTCCTAGACCTCCACGTGCCGT | |
| | GCAGTACAGCCACACCCACACCACCAACTACAAGCTGAGCAGCGAGGTGC | |
| | ACAACGACGTGGCCATCAGACCCCGGACCAACCTGACCACCGTG | |
| 3′ UTR | TGATAATAGGCTGGAGCCTCGGTGGCCTAGCTTCTTGCCCCTTGGGCCTCC | 4 |
| | CCCCAGCCCCTCCTCCCCTTCCTGCACCCGTACCCCCGTGGTCTTTGAATAA | |
| | AGTCTGAGTGGGCGGC | |
| Corresponding | MGAQVSRQNVGTHSTQNSVSNGSSLNYFNINYFKDAASSGASRLDFSQDPSKF | 5 |
| Amino Acid | TDPVKDVLEKGIPTLQSPSVEACGYSDRIIQITRGDSTITSQDVANAVVGYGVW | |
| Sequence | PHYLTPQDATAIDKPTQPDTSSNRFYTLDSKMWNSTSKGWWWKLPDALKDM | |
| | GIFGENMFYHFLGRSGYTVHVQCNASKFHQGTLLVVMIPEHQLATVNKGNVN | |
| | AGYKYTHPGEAGREVGTQVENEKQPSDDNWLNFDGTLLGNLLIFPHQFINLRS | |
| | NNSATLIVPYVNAVPMDSMVRHNNWSLVIIPVCQLQSNNISNIVPITVSISPMC | |
| | AEFSGARAKTVVQGLPVYVTPGSGQFMTTDDMQSPCALPWYHPTKEIFIPGEV | |
| | KNLIEMCQVDTLIPINSTQSNIGNVSMYTVTLSPQTKLAEEIFAIKVDIASHPLA | |
| | TTLIGEIASYFTHWTGSLRFSFMFCGTANTTLKVLLAYTPPGIGKPRSRKEAML | |
| | GTHVVWDVGLQSTVSLVVPWISASQYRFTTPDTYSSAGYITCWYQTNFVVPP | |
| | NTPNTAEMLCFVSGCNHFCLRMARDTDLHKQTGPITQNPVENYIDEVLNEVL | |
| | VVPNINQSHPTTSNAAPVLDAAETGHTNKIQPEDTIETRYVQSSQTLDEMSVES | |
| | FLGRSGCIHESVLDIVDNYNDQSFTKWNINLQEMAQIRRKFEMFTYARFDSEIT | |
| | MVPSVAAKDGHIGHIVMQYMYVPPGAPIPTTRDDYAWQSGTNASVFWQHGQ | |
| | PFPRFSLPFLSIASAYYMFYDGYDGDTYKSRYGTVVTNDMGTLCSRIVTSEQL | |
| | HKVKVVTRIYHKAKHTKAWCPRPPRAVQYSHTHTTNYKLSSEVHNDVAIRPR | |
| | TNLTTV | |
| Poly A tail | 100 nt | |

HRV-A16 3CD protease

| Chemistry: 1-methylpseudouridine | | |
|---|---|---|
| Cap: 7mG(5′)ppp(5′)NlmpNp | | |
| SEQ ID NO: 6 consists of from 5′ end to 3′ end: 5′ UTR SEQ ID NO: 2, | | 6 |
| mRNA ORF SEQ ID NO: 7, and 3′ UTR SEQ ID NO: 4. | | |
| 5′ UTR | GGGAAATAAGAGAGAAAAGAAGAGTAAGAAGAAATATAAGACCCCGGCG | 2 |
| | CCGCCACC | |
| ORF of | ATGGGACCAGAAGAGGAATTCGGAATGTCCATCATTAAGAACAACACCTG | 7 |
| mRNA | TGTGGTCACCACCACTAACGGGAAGTTCACCGGCCTGGGAATCTACGACA | |
| Construct | GAATTCTCATTCTTCCGACTCACGCTGACCCGGGAAGCGAAATTCAAGTCA | |
| (excluding | ACGGTATTCACACCAAGGTCCTCGATTCCTACGACCTGTTTAACAAGGAAG | |
| stop codon) | GGGTCAAGCTGGAAATCACCGTGCTGAAGCTGGACCGGAACGAGAAGTTT | |
| | CGGGATATCCGCAAGTACATCCCCGAATCCGAGGACGACTACCCGGAGTG | |
| | CAACCTGGCACTGGTGGCCAATCAAACCGAACCCACCATCATCAAAGTCG | |
| | GCGACGTGGTGTCCTACGGAAACATCCTGCTTTCCGGAACTCAGACGGCCC | |
| | GCATGCTGAAGTATAACTACCCAACCAAGTCCGGGTACTGCGGCGGTGTCC | |
| | TGTACAAGATCGGTCAGATCCTCGGAATCCACGTCGGAGGAAACGGCCGC | |
| | GACGGGTTCTCCTCCATGCTGTTGAGGAGCTACTTCACTGAGCAACAGGGG | |
| | CAGATCCAGATCAGCAAACACGTGAAGGACGTGGGCCTTCCCTCCATTCAC | |
| | ACTCCTACTAAGACCAAGCTGCAGCCTTCCGTGTTCTACGACATTTTCCCC | |
| | GGATCCAAGGAGCCCGCCGTGCTGACCGAGAAGGATCCAAGACTGAAAGT | |
| | GGACTTCGACTCTGCCCTGTTCTCAAAGTACAAGGGAAACACTGAGTGCAG | |
| | CCTTAACGAGCACATTCAGGTCGCGGTGGCCCACTACTCGGCCCAACTGGC | |
| | TACCCTGGACATCGACCCTCAGCCGATCGCGATGGAAGATTCAGTGTTCGG | |
| | AATGGACGGCCTGGAAGCACTGGACTTGAACACCTCCGCGGGCTACCCCT | |
| | ACGTGACCCTCGGTATCAAGAAGAAGGACCTGATTAACAACAAGACCAAA | |
| | GATATTTCGAAGCTCAAGCTCGCGCTGGATAAGTACGACGTGGACTTGCCG | |
| | ATGATCACCTTCCTGAAAGACGAACTGAGGAAGAAGGATAAGATCGCCGC | |
| | CGGCAAGACTAGAGTCATCGAAGCATCCAGCATTAACGACACCATTCTGTT | |
| | CCGGACTGTGTACGGGAACCTGTTCTCGAAGTTCCATCTGAACCCCGGCGT | |
| | GGTCACCGGCTGCGCCGTGGGCTGCGACCCGGAAACTTTCTGGAGCAAGA | |
| | TCCCTCTCATGCTGGACGGAGACTGTATTATGGCCTTCGACTATACCAACT | |
| | ACGACGGCAGCATTCATCCTATCTGGTTCAAGGCCCTGGGAATGGTGCTGG | |
| | ACAACCTGTCGTTCAATCCGACCCTGATCAACCGGCTGTGTAACTCCAAGC | |
| | ACATTTTCAAGTCGACCTACTACGAAGTGGAGGGCGGAGTGCCGTCCGGCT | |
| | GCTCGGGCACTAGCATTTTCAACTCCATGATCAACAATATCATTATCCGCA | |
| | CCCTGGTCCTCGACGCCTACAAGCACATCGACCTCGATAAGCTCAAGATCA | |
| | TCGCCTACGGCGACGACGTGATTTTCTCATATAAGTACAAATTGGACATGG | |
| | AGGCCATCGCTAAGGAGGGACAGAAATACGGTCTGACTATCACACCCGCC | |
| | GATAAGAGCTCAGAGTTCAAGGAACTCGACTACGGCAACGTGACGTTTCT | |
| | GAAGCGCGGCTTTAGACAGGACGACAAGTACAAGTTCCTCATCCACCCTA | |
| | CTTTCCCGGTGGAGGAGATCTACGAGTCCATCCGGTGGACTAAGAAGCCGT | |
| | CCCAGATGCAGGAACACGTGCTCTCCCTGTGCCACCTCATGTGGCACAACG | |
| | GCCCTGAGATCTATAAGGACTTTGAGACAAAGATACGCTCTGTGTCCGCAG | |
| | GACGGGCGCTTTACATCCCTCCGTACGAGCTGCTGCGGCACGAGTGGTACG | |
| | AGAAGTTC | |
| 3′ UTR | TGATAATAGGCTGGAGCCTCGGTGGCCTAGCTTCTTGCCCCTTGGGCCTCC | 4 |
| | CCCCAGCCCCTCCTCCCCTTCCTGCACCCGTACCCCCGTGGTCTTTGAATAA | |
| | AGTCTGAGTGGGCGGC | |
| Corresponding | MGPEEEFGMSIIKNNTCVVTTTNGKFTGLGIYDRILILPTHADPGSEIQVNGIHT | 8 |
| Amino Acid | KVLDSYDLFNKEGVKLEITVLKLDRNEKFRDIRKYIPESEDDYPECNLALVAN | |
| Sequence | QTEPTIIKVGDVVSYGNILLSGTQTARMLKYNYPTKSGYCGGVLYKIGQILGIH | |
| | VGGNGRDGFSSMLLRSYFTEQQGQIQISKHVKDVGLPSIHTPTKTKLQPSVFYD | |
| | IFPGSKEPAVLTEKDPRLKVDFDSALFSKYKGNTECSLNEHIQVAVAHYSAQL | |
| | ATLDIDPQPIAMEDSVFGMDGLEALDLNTSAGYPYVTLGIKKKDLINNKTKDIS | |
| | KLKLALDKYDVDLPMITFLKDELRKKDKIAAGKTRVIEASSINDTILFRTVYGN | |
| | LFSKFHLNPGVVTGCAVGCDPETFWSKIPLMLDGDCIMAFDYTNYDGSIHPIW | |
| | FKALGMVLDNLSFNPTLINRLCNSKHIFKSTYYEVEGGVPSGCSGTSIFNSMIN | |
| | NIIIRTLVLDAYKHIDLDKLKIIAYGDDVIFSYKYKLDMEAIAKEGQKYGLTITP | |
| | ADKSSEFKELDYGNVTFLKRGFRQDDKYKFLIHPTFPVEEIYESIRWTKKPSQM | |
| | QEHVLSLCHLMWHNGPEIYKDFETKIRSVSAGRALYIPPYELLRHEWYEKF | |
| Poly A tail | 100 nt | |

HRV-B14 P1

| Chemistry: 1-methylpseudouridine | | |
|---|---|---|
| Cap: 7mG(5′)ppp(5′)NlmpNp | | |
| SEQ ID NO: 9 consists of from 5′ end to 3′ end: 5′ UTR SEQ ID NO: 2, | | 9 |
| mRNA ORF SEQ ID NO: 10, and 3′ UTR SEQ ID NO: 4. | | |
| 5′ UTR | GGGAAATAAGAGAGAAAAGAAGAGTAAGAAGAAATATAAGACCCCGGCG | 2 |
| | CCGCCACC | |
| ORF of | ATGGGCGCCCAGGTGAGCACCCAGAAGTCGGGCAGCCACGAGAACCAGA | 10 |
| mRNA | ACATCCTGACCAACGGCAGCAACCAGACCTTCACCGTGATCAACTACTACA | |
| Construct | AGGACGCCGCCAGCACCAGCAGCGCCGGCCAGAGCCTGAGCATGGACCCT | |
| (excluding | AGCAAGTTCACCGAGCCTGTGAAGGACCTGATGCTGAAGGGCGCCCCTGC | |
| stop codon) | CCTGAACAGCCCTAACGTGGAGGCCTGCGGCTACAGCGACAGAGTGCAGC | |
| | AGATCACCCTGGGCAACAGCACCATCACCACCCAGGAGGCCGCCAACGCC | |
| | GTGGTGTGCTACGCCGAGTGGCCTGAGTACCTGCCTGACGTGGACGCCAGC | |
| | GACGTGAACAAGACCAGCAAGCCTGACACCAGCGTGTGCAGATTCTACAC | |
| | CCTGGACAGCAAGACCTGGACCACTGGCTCCAAGGGCTGGTGCTGGAAGC | |
| | TTCCTGACGCCCTCAAGGACATGGGCGTGTTCGGACAGAATATGTTCTTCC | |
| | ACAGCCTGGGCAGAAGCGGTTACACCGTGCACGTGCAGTGCAACGCCACC | |
| | AAGTTCCATTCCGGCTGCCTGCTGGTGGTGGTGATCCCTGAGCACCAACTG | |
| | GCTTCGCACGAAGGCGGCAACGTGAGCGTGAAGTACACGTTCACTCACCC | |
| | AGGCGAGAGAGGCATCGACCTGTCAAGCGCAAACGAGGTGGGCGGCCCTG | |
| | TTAAGGACGTGATCTACAACATGAACGGCACCCTGTTAGGTAATCTGCTGA | |
| | TCTTCCCTCACCAGTTCATCAACCTGAGAACCAACAACACCGCCACCATCG | |
| | TCATTCCATACATCAACAGCGTGCCTATCGACAGCATGACCAGACACAACA | |
| | ACGTCAGTCTGATGGTAATCCCAATCGCCCCTCTGACCGTGCCTACCGGCG | |
| | CCACCCCTAGCCTGCCTATAACCGTGACCATCGCTCCTATGTGCACCGAGT | |
| | TCAGCGGCATCAGAAGCAAGAGCATCGTGCCTCAGGGTCTACCGACCACC | |
| | ACCCTGCCTGGCAGCGGCCAGTTCCTAACTACCGACGACAGACAGAGCCC | |
| | TAGCGCCCTGCCAAATTACGAGCCTACCCCTAGAATCCACATCCCTGGCAA | |
| | GGTGCACAACCTGCTGGAGATCATCCAGGTGGACACCTTGATCCCGATGA | |
| | ATAATACCCACACCAAGGACGAGGTGAACAGCTATCTGATACCGCTGAAC | |
| | GCCAATAGGCAGAACGAGCAGGTGTTCGGCACCAACCTGTTCATCGGCGA | |
| | CGGAGTGTTCAAGACAACCCTGCTGGGAGAGATCGTGCAGTACTACACCC | |
| | ACTGGTCTGGAAGTCTGAGATTCAGTCTTATGTACACCGGTCCTGCACTAA | |
| | GCTCTGCCAAGCTGATCCTGGCCTACACCCCTCCTGGCGCCAGAGGCCCTC | |
| | AGGACAGAAGAGAGGCCATGCTAGGCACCCACGTCGTTTGGGACATCGGC | |
| | CTGCAGTCAACAATTGTAATGACCATCCCTTGGACCAGCGGCGTGCAGTTC | |
| | AGATACACCGACCCTGATACTTACACATCCGCAGGCTTCTTGAGCTGCTGG | |
| | TATCAGACCTCACTGATCCTTCCTCCTGAGACAACTGGCCAGGTGTATCTG | |
| | CTATCGTTCATCAGCGCTTGCCCTGACTTCAAGCTGAGACTGATGAAGGAT | |
| | ACCCAGACCATCAGCCAGACCGTGGCCCTGACCGAGGGCCTGGGCGACGA | |
| | GCTGGAGGAGGTGATCGTGGAGAAGACCAAGCAGACGGTGGCCAGCATCT | |
| | CTAGCGGACCTAAGCATACACAGAAGGTCCCAATCTTGACTGCTAACGAG | |
| | ACAGGAGCCACTATGCCTGTGCTGCCTTCCGACAGTATCGAAACAAGAAC | |
| | CACCTACATGCACTTCAACGGATCTGAAACAGACGTGGAGTGCTTCCTTGG | |
| | ACGAGCCGCCTGCGTCCACGTAACCGAGATCCAGAACAAGGACGCCACCG | |
| | GAATTGACAACCACAGGGAGGCAAAGTTGTTCAACGACTGGAAGATTAAC | |
| | TTGAGTAGCCTGGTGCAGCTGAGAAAGAAGCTGGAGTTGTTCACCTACGTG | |
| | AGATTCGACAGCGAGTACACCATTCTGGCAACCGCCTCCCAGCCAGATTCC | |
| | GCCAACTACTCTTCCAACCTGGTTGTGCAGGCCATGTACGTACCGCCGGGA | |
| | GCCCCGAACCCTAAGGAGTGGGACGACTACACCTGGCAGAGCGCCAGCAA | |
| | CCCTAGCGTGTTCTTCAAGGTTGGCGATACCTCCAGGTTCAGTGTCCCATA | |
| | CGTGGGCCTGGCGTCCGCCTACAACTGCTTCTACGACGGATACAGCCACGA | |
| | CGACGCCGAAACACAGTACGGCATCACCGTACTGAACCACATGGGCAGCA | |
| | TGGCCTTCAGAATCGTTAACGAACACGACGAGCACAAGACCCTGGTGAAG | |
| | ATCAGAGTGTATCACAGAGCCAAGCACGTCGAGGCCTGGATCCCTAGAGC | |
| | ACCACGGGCACTACCATATACTAGCATCGGCAGAACCAATTACCCTAAGA | |
| | ACACAGAACCAGTGATCAAGAAGAGAAAGGGCGACATCAAGAGCTAC | |
| 3′ UTR | TGATAATAGGCTGGAGCCTCGGTGGCCTAGCTTCTTGCCCCTTGGGCCTCC | 4 |
| | CCCCAGCCCCTCCTCCCCTTCCTGCACCCGTACCCCCGTGGTCTTTGAATAA | |
| | AGTCTGAGTGGGCGGC | |
| Corresponding | MGAQVSTQKSGSHENQNILTNGSNQTFTVINYYKDAASTSSAGQSLSMDPSKF | 11 |
| Amino Acid | TEPVKDLMLKGAPALNSPNVEACGYSDRVQQITLGNSTITTQEAANAVVCYA | |
| Sequence | EWPEYLPDVDASDVNKTSKPDTSVCRFYTLDSKTWTTGSKGWCWKLPDALK | |
| | DMGVFGQNMFFHSLGRSGYTVHVQCNATKFHSGCLLVVVIPEHQLASHEGG | |
| | NVSVKYTFTHPGERGIDLSSANEVGGPVKDVIYNMNGTLLGNLLIFPHQFINLR | |
| | TNNTATIVIPYINSVPIDSMTRHNNVSLMVIPIAPLTVPTGATPSLPITVTIAPMC | |
| | TEFSGIRSKSIVPQGLPTTTLPGSGQFLTTDDRQSPSALPNYEPTPRIHIPGKVHN | |
| | LLEIIQVDTLIPMNNTHTKDEVNSYLIPLNANRQNEQVFGTNLFIGDGVFKTTL | |
| | LGEIVQYYTHWSGSLRFSLMYTGPALSSAKLILAYTPPGARGPQDRREAMLGT | |
| | HVVWDIGLQSTIVMTIPWTSGVQFRYTDPDTYTSAGFLSCWYQTSLILPPETTG | |
| | QVYLLSFISACPDFKLRLMKDTQTISQTVALTEGLGDELEEVIVEKTKQTVASIS | |
| | SGPKHTQKVPILTANETGATMPVLPSDSIETRTTYMHENGSETDVECFLGRAA | |
| | CVHVTEIQNKDATGIDNHREAKLFNDWKINLSSLVQLRKKLELFTYVREDSEY | |
| | TILATASQPDSANYSSNLVVQAMYVPPGAPNPKEWDDYTWQSASNPSVFFKV | |
| | GDTSRFSVPYVGLASAYNCFYDGYSHDDAETQYGITVLNHMGSMAFRIVNEH | |
| | DEHKTLVKIRVYHRAKHVEAWIPRAPRALPYTSIGRTNYPKNTEPVIKKRKGD | |
| | IKSY | |
| PolyA tail | 100 nt | |

HRV-B14 3CD protease

| Chemistry: 1-methylpseudouridine | | |
|---|---|---|
| Cap: 7mG(5′)ppp(5′)NlmpNp | | |
| SEQ ID NO: 12 consists of from 5′ end to 3′ end: 5′ UTR SEQ ID NO: 2, | | 12 |
| mRNA ORF SEQ ID NO: 13, and 3′ UTR SEQ ID NO: 4. | | |
| 5′ UTR | GGGAAATAAGAGAGAAAAGAAGAGTAAGAAGAAATATAAGACCCCGGCG | 2 |
| | CCGCCACC | |
| ORF of | ATGGGCCCTAACACCGAGTTCGCCCTGAGCCTGCTGAGAAAGAACATCAT | 13 |
| mRNA | GACCATCACCACCAGCAAGGGCGAGTTCACCGGCCTGGGCATCCACGACA | |
| Construct | GAGTGTGCGTGATCCCTACCCACGCCCAGCCTGGCGACGACGTGCTGGTGA | |
| (excluding | ACGGCCAGAAGATCAGAGTGAAGGACAAGTACAAGCTGGTGGACCCTGAG | |
| stop codon) | AACATTAACCTGGAGCTGACCGTGCTGACCCTGGACAGAAACGAGAAGTT | |
| | CAGAGACATCAGAGGCTTCATCAGCGAGGACCTGGAGGGCGTGGACGCCA | |
| | CCCTGGTGGTGCACAGCAACAACTTCACCAACACCATCCTGGAGGTGGGC | |
| | CCTGTGACCATGGCCGGCCTGATTAATTTGAGCAGCACCCCTACCAACAGA | |
| | ATGATCAGATACGACTACGCCACCAAGACCGGCCAGTGCGGCGGCGTGCT | |
| | GTGCGCCACCGGCAAGATCTTCGGTATACACGTGGGCGGCAACGGCAGAC | |
| | AGGGCTTCAGCGCCCAGCTGAAGAAGCAGTACTTCGTGGAGAAGCAGGGC | |
| | CAGGTGATCGCCAGACACAAGGTGAGAGAGTTCAATATCAATCCTGTGAA | |
| | CACACCAACTAAGAGCAAGCTGCACCCTAGCGTGTTCTACGACGTGTTCCC | |
| | AGGAGACAAGGAGCCTGCAGTCCTGAGCGACAACGACCCTAGACTTGAAG | |
| | TCAAGCTTACTGAGAGCCTGTTCTCTAAGTATAAGGGCAACGTCAACACAG | |
| | AGCCTACCGAGAACATGCTGGTGGCCGTGGACCACTACGCCGGCCAGCTG | |
| | CTATCTCTGGACATCCCAACAAGCGAGCTGACGTTGAAGGAGGCCCTGTAC | |
| | GGCGTCGACGGCCTGGAGCCTATCGATATCACGACCAGCGCCGGCTTCCCT | |
| | TACGTGTCCCTGGGTATCAAGAAGAGGGATATTCTGAACAAGGAGACTCA | |
| | GGACACCGAGAAGATGAAGTTCTACCTCGATAAGTACGGCATCGACCTGC | |
| | CTCTGGTGACCTACATCAAGGACGAGCTGAGAAGCGTGGACAAGGTCCGC | |
| | TTGGGCAAGAGCAGACTGATCGAGGCCAGCAGCCTGAACGACAGCGTGAA | |
| | CATGAGAATGAAGCTGGGCAACCTGTACAAGGCCTTCCACCAGAACCCTG | |
| | GAGTATTGACCGGCAGCGCCGTGGGCTGCGACCCTGACGTGTTCTGGAGC | |
| | GTTATCCCTTGCCTGATGGACGGACACCTGATGGCCTTCGACTACAGCAAC | |
| | TTCGACGCCAGCCTGAGCCCTGTGTGGTTCGTGTGCCTGGAGAAGGTCCTG | |
| | ACCAAGCTGGGTTTCGCCGGTTCCTCTTTGATCCAGAGCATCTGCAACACC | |
| | CACCACATCTTCCGGGACGAGATCTACGTGGTAGAGGGTGGCATGCCTAG | |
| | CGGCTGCAGCGGCACCAGCATCTTCAACAGCATGATCAACAACATCATCAT | |
| | TAGAACACTGATCCTGGACGCCTATAAGGGAATTGACCTGGATAAGTTGA | |
| | AGATCCTGGCCTACGGAGACGACCTGATCGTGAGCTACCCTTACGAGCTGG | |
| | ACCCTCAGGTGCTGGCAACCCTCGGAAAGAACTACGGCTTGACAATCACG | |
| | CCTCCTGACAAGAGCGAGACATTCACAAAGATGACTTGGGAGAACCTGAC | |
| | CTTCCTGAAGAGATACTTCAAGCCTGACCAGCAGTTCCCTTTCCTGGTGCA | |
| | CCCTGTCATGCCTATGAAGGATATTCACGAGAGCATCCGGTGGACCAAGG | |
| | ACCCTAAGAACACTCAGGACCACGTGAGAAGCCTGTGCATGCTGGCCTGG | |
| | CACAGCGGCGAGAAGGAGTACAACGAGTTCATTCAGAAGATTAGGACTAC | |
| | CGACATCGGCAAGTGCCTGATTCTTCCTGAGTACTCAGTGCTTAGGAGGCG | |
| | CTGGCTGGACCTGTTC | |
| 3′ UTR | TGATAATAGGCTGGAGCCTCGGTGGCCTAGCTTCTTGCCCCTTGGGCCTCC | 4 |
| | CCCCAGCCCCTCCTCCCCTTCCTGCACCCGTACCCCCGTGGTCTTTGAATAA | |
| | AGTCTGAGTGGGCGGC | |
| Corresponding | MGPNTEFALSLLRKNIMTITTSKGEFTGLGIHDRVCVIPTHAQPGDDVLVNGQ | 14 |
| Amino Acid | KIRVKDKYKLVDPENINLELTVLTLDRNEKFRDIRGFISEDLEGVDATLVVHSN | |
| Sequence | NFTNTILEVGPVTMAGLINLSSTPTNRMIRYDYATKTGQCGGVLCATGKIFGIH | |
| | VGGNGRQGFSAQLKKQYFVEKQGQVIARHKVREFNINPVNTPTKSKLHPSVF | |
| | YDVFPGDKEPAVLSDNDPRLEVKLTESLFSKYKGNVNTEPTENMLVAVDHYA | |
| | GQLLSLDIPTSELTLKEALYGVDGLEPIDITTSAGFPYVSLGIKKRDILNKETQD | |
| | TEKMKFYLDKYGIDLPLVTYIKDELRSVDKVRLGKSRLIEASSLNDSVNMRMK | |
| | LGNLYKAFHQNPGVLTGSAVGCDPDVFWSVIPCLMDGHLMAFDYSNEDASLS | |
| | PVWFVCLEKVLTKLGFAGSSLIQSICNTHHIFRDEIYVVEGGMPSGCSGTSIENS | |
| | MINNIIIRTLILDAYKGIDLDKLKILAYGDDLIVSYPYELDPQVLATLGKNYGLT | |
| | ITPPDKSETFTKMTWENLTFLKRYFKPDQQFPFLVHPVMPMKDIHESIRWTKD | |
| | PKNTQDHVRSLCMLAWHSGEKEYNEFIQKIRTTDIGKCLILPEYSVLRRRWLD | |
| | LF | |
| Poly A tail | 100 nt | |

EV-D68 isolate US/MO/14-18950 P1

| Chemistry: 1-methylpseudouridine | | |
|---|---|---|
| Cap: 7mG(5′)ppp(5′)NlmpNp | | |
| SEQ ID NO: 15 consists of from 5′ end to 3′ end: 5′ UTR SEQ ID NO: 2, | | 15 |
| mRNA ORF SEQ ID NO: 16, and 3′ UTR SEQ ID NO: 4. | | |
| 5′ UTR | GGGAAATAAGAGAGAAAAGAAGAGTAAGAAGAAATATAAGACCCCGGCG | 2 |
| | CCGCCACC | |
| ORF of | ATGGGCGCCCAGGTGACGCGCCAGCAGACCGGCACCCACGAGAACGCCAA | 16 |
| mRNA | CATCGCCACCAACGGCAGCCACATCACCTATAACCAGATCAACTTCTATAA | |
| Construct | GGACTCTTACGCAGCCAGCGCCAGCAAGCAGGACTTTAGCCAGGACCCTT | |
| (excluding | CAAAGTTCACCGAGCCTGTGGTGGAGGGCCTGAAGGCCGGCGCACCCGTG | |
| stop codon) | CTGAAGTCCCCTAGCGCAGAGGCCTGCGGATACAGCGACAGAGTGCTTCA | |
| | GCTGAAACTGGGCAATAGCGCTATCGTGACCCAGGAAGCGGCCAACTACT | |
| | GTTGCGCCTACGGTGAGTGGCCTAACTATCTGCCAGACCACGAGGCCGTGG | |
| | CGATCGATAAACCTACTCAGCCTGAGACAGCGACAGACAGATTCTACACC | |
| | TTAAAGAGTGTGAAGTGGGAAACCGGCAGCACCGGCTGGTGGTGGAAGCT | |
| | ACCTGACGCCCTGAACAACATCGGCATGTTCGGCCAGAACGTGCAGCATC | |
| | ACTACCTCTACCGGAGCGGCTTCCTGATTCACGTGCAGTGCAACGCCACCA | |
| | AGTTTCACCAGGGAGCCCTGCTGGTCGTAGCAATCCCTGAGCATCAAAGA | |
| | GGCGCCCACAACACCAACACCAGCCCTGGCTTTGACGACATCATGAAGGG | |
| | CGAAGAGGGCGGCACATTCAACCACCCTTACGTGCTGGACGACGGCACCA | |
| | GCCTGGCCTGCGCGACCATCTTCCCTCACCAGTGGATCAACCTCAGAACCA | |
| | ACAACTCTGCTACCATCGTGCTGCCTTGGATGAACGCGGCCCCTATGGACT | |
| | TCCCTCTGAGACACAATCAGTGGACCCTCGCCATTATCCCTGTGGTGCCCC | |
| | TGGGCACCAGAACCACCAGCAGCATGGTGCCTATCACCGTCAGCATTGCCC | |
| | CAATGTGCTGCGAATTCAACGGCTTGCGGCACGCCATCACACAGGGCGTG | |
| | CCCACCTACCTGCTGCCCGGTAGCGGCCAGTTTCTGACCACCGACGACCAC | |
| | TCGTCTGCACCTGCTCTCCCTTGCTTCAATCCTACCCCAGAGATGCACATCC | |
| | CTGGCCAGGTGAGAAACATGCTGGAGGTGGTGCAGGTGGAGAGCATGATG | |
| | GAAATCAACAACACCGAAAGCGCCGTCGGCATGGAGCGCCTGAAAGTGGA | |
| | CATCTCCGCGCTGACTGACGTGGACCAGCTGCTGTTCAACATCCCTCTGGA | |
| | CATCCAGCTGGACGGGCCTCTTCGCAACACCCTGGTTGGCAACATCAGCAG | |
| | ATACTACACTCACTGGAGTGGCAGCCTGGAGATGACCTTCATGTTCTGCGG | |
| | AAGTTTCATGGCCACCGGCAAGCTGATCCTTTGCTACACTCCTCCTGGCGG | |
| | CAGCTGTCCTACCACACGGGAGACTGCCATGCTGGGAACCCACATCGTGTG | |
| | GGACTTCGGGCTGCAGAGCAGCGTGACCCTGATCATCCCTTGGATCAGCGG | |
| | CTCTCACTACAGAATGTTTAACAACGACGCTAAGAGCACCAACGCCAACG | |
| | TGGGCTACGTGACCTGCTTCATGCAGACAAACCTGATAGTGCCTAGCGAGT | |
| | CAAGCGACACCTGCAGCCTGATCGGGTTCATCGCCGCCAAAGACGACTTTA | |
| | GCCTTAGACTGATGCGAGACAGCCCTGATATCGGGCAGCTTGACCACCTGC | |
| | ACGCCGCCGAGGCAGCTTACCAAATCGAGAGCATTATCAAGACCGCCACC | |
| | GATACCGTCAAGAGTGAGATCAACGCCGAACTGGGCGTTGTGCCTAGCCT | |
| | GAACGCCGTGGAGACAGGCGCCACCAGCAACACCGAGCCTGAGGAGGCC | |
| | ATACAGACCAGAACCGTGATCAATCAGCACGGCGTGAGCGAGACACTGGT | |
| | CGAGAACTTCTTGAGCAGAGCTGCCCTCGTGAGCAAGAGATCTTTCGAGTA | |
| | CAAGGACCACACCTCTTCCGCCGCCCAAGCCGACAAGAACTTCTTCAAGTG | |
| | GACCATCAACACTAGGAGCTTCGTGCAGCTAAGAAGGAAGCTGGAGCTGT | |
| | TTACTTACCTGAGATTCGACGCTGAGATCACCATTCTGACGACCGTGGCCG | |
| | TGAACGGCAGCAGCAATAACACATACGTCGGACTGCCTGATCTGACGCTG | |
| | CAGGCCATGTTCGTGCCTACCGGCGCCCTGACACCTGAGAAGCAAGACAG | |
| | CTTCCACTGGCAGAGCGGCAGCAACGCAAGCGTGTTCTTCAAGATCAGCG | |
| | ATCCTCCTGCACGCATCACCATCCCATTCATGTGCATCAATAGCGCCTATTC | |
| | CGTGTTCTACGACGGCTTCGCCGGCTTCGAGAAGAACGGCCTTTACGGCAT | |
| | CAATCCCGCCGACACTATCGGCAACCTGTGCGTGAGAATCGTGAACGAGC | |
| | ACCAGCCTGTCGGGTTCACTGTGACCGTTAGAGTGTACATGAAGCCTAAGC | |
| | ACATCAAGGCCTGGGCCCCTCGTCCACCTAGAACCCTGCCTTACATGTCTA | |
| | TTGCTAACGCCAACTACAAGGGCAAGGAGAGAGCCCCTAACGCGCTCTCA | |
| | GCCATCATTGGCAATCGTGACAGCGTGAAGACCATGCCTCACAACATTGTG | |
| | AACACC | |
| 3′ UTR | TGATAATAGGCTGGAGCCTCGGTGGCCTAGCTTCTTGCCCCTTGGGCCTCC | 4 |
| | CCCCAGCCCCTCCTCCCCTTCCTGCACCCGTACCCCCGTGGTCTTTGAATAA | |
| | AGTCTGAGTGGGCGGC | |
| Corresponding | MGAQVTRQQTGTHENANIATNGSHITYNQINFYKDSYAASASKQDFSQDPSKF | 17 |
| Amino Acid | TEPVVEGLKAGAPVLKSPSAEACGYSDRVLQLKLGNSAIVTQEAANYCCAYG | |
| Sequence | EWPNYLPDHEAVAIDKPTQPETATDRFYTLKSVKWETGSTGWWWKLPDALN | |
| | NIGMFGQNVQHHYLYRSGFLIHVQCNATKFHQGALLVVAIPEHQRGAHNTNT | |
| | SPGFDDIMKGEEGGTFNHPYVLDDGTSLACATIFPHQWINLRTNNSATIVLPW | |
| | MNAAPMDFPLRHNQWTLAIIPVVPLGTRTTSSMVPITVSIAPMCCEFNGLRHAI | |
| | TQGVPTYLLPGSGQFLTTDDHSSAPALPCFNPTPEMHIPGQVRNMLEVVQVES | |
| | MMEINNTESAVGMERLKVDISALTDVDQLLFNIPLDIQLDGPLRNTLVGNISRY | |
| | YTHWSGSLEMTFMFCGSFMATGKLILCYTPPGGSCPTTRETAMLGTHIVWDF | |
| | GLQSSVTLIIPWISGSHYRMENNDAKSTNANVGYVTCFMQTNLIVPSESSDTCS | |
| | LIGFIAAKDDFSLRLMRDSPDIGQLDHLHAAEAAYQIESIIKTATDTVKSEINAE | |
| | LGVVPSLNAVETGATSNTEPEEAIQTRTVINQHGVSETLVENFLSRAALVSKRS | |
| | FEYKDHTSSAAQADKNFFKWTINTRSFVQLRRKLELFTYLRFDAEITILTTVAV | |
| | NGSSNNTYVGLPDLTLQAMFVPTGALTPEKQDSFHWQSGSNASVFFKISDPPA | |
| | RITIPFMCINSAYSVFYDGFAGFEKNGLYGINPADTIGNLCVRIVNEHQPVGFTV | |
| | TVRVYMKPKHIKAWAPRPPRTLPYMSIANANYKGKERAPNALSAIIGNRDSV | |
| | KTMPHNIVNT | |
| Poly A tail | 100 nt | |

EV-D68 US/MO/14-18950 isolate 3CD protease

| Chemistry: 1-methylpseudouridine | | |
|---|---|---|
| Cap: 7mG(5′)ppp(5′)NlmpNp | | |
| SEQ ID NO: 18 consists of from 5′ end to 3′ end: 5′ UTR SEQ ID NO: 2, | | 18 |
| mRNA ORF SEQ ID NO: 19, and 3′ UTR SEQ ID NO: 4 | | |
| 5′ UTR | GGGAAATAAGAGAGAAAAGAAGAGTAAGAAGAAATATAAGACCCCGGCG | 2 |
| | CCGCCACC | |
| ORF of | ATGGGCCCTGGCTTCGACTTCGCCCAGGCCATCATGAAGAAGAACACCGT | 19 |
| mRNA | GATCGCCAGAACCGAGAAGGGCGAGTTCACCATGCTGGGCGTGTACGACA | |
| Construct | GAGTGGCCGTGATCCCTACCCACGCCAGCGTGGGCGAAACCATCTACATC | |
| (excluding | AACGACGTGGAAACGAAGGTGCTGGACGCCTGCGCCCTGCGCGACCTGAC | |
| stop codon) | CGACACCAACCTGGAGATCACCATCGTGAAGCTGGACAGAAACCAGAAGT | |
| | TCAGAGACATCAGACACTTCCTGCCTAGATACGAGGACGACTACAACGAC | |
| | GCCGTGCTGAGCGTGCACACCAGCAAGTTCCCTAACATGTACATCCCTGTG | |
| | GGCCAGGTGACCAACTACGGCTTCCTGAACCTGGGCGGCACCCCAACTCAT | |
| | AGAATCCTGATGTACAACTTCCCTACCAGAGCCGGCCAGTGCGGCGGCGT | |
| | GGTGACCACCACCGGCAAGGTGATCGGCATCCACGTGGGCGGAAACGGCG | |
| | CCCAGGGCTTCGCCGCCATGCTGCTGCACAGCTACTTCACCGATACCCAGG | |
| | GCGAGATCGTGAGCAGCGAGAAGTCCGGCGTGTGCATCAACGCCCCTGCC | |
| | AAGACCAAGCTGCAGCCTAGCGTGTTCCACCAGGTGTTCGAGGGCAGCAA | |
| | GGAGCCTGCAGTACTGAACCCTAAGGACCCTAGACTGAAGACCGACTTCG | |
| | AGGAGGCCATCTTCAGCAAGTATACTGGTAACAAGATCATGCTGATGGAC | |
| | GAGTACATGGAAGAGGCGGTGGACCACTACGTGGGCTGCCTGGAGCCTCT | |
| | GGACATCAGCGTGGACCCTATCCCTCTGGAGAGCGCCATGTACGGCATGG | |
| | ACGGCCTGGAGGCCCTGGACTTGACTACTAGCGCCGGCTTCCCTTACCTGC | |
| | TGCAGGGCAAGAAGAAGCGAGACATCTTCAACAGACACACCAGAGACACC | |
| | AGCGAGATGACCAAGATGCTGGAGAAGTACGGTGTGGATCTGCCTTTCGT | |
| | GACCTTCGTGAAGGACGAGCTGAGAAGCAGAGAGAAGGTGGAGAAGGGT | |
| | AAGAGCAGACTGATCGAGGCCAGCAGCCTGAACGACAGCGTGGCCATGAG | |
| | AGTGGCTTTCGGCAACCTGTACGCCACCTTCCACAACAACCCTGGCACCGC | |
| | TACGGGTAGCGCCGTGGGTTGCGACCCTGATATCTTCTGGAGCAAGATCCC | |
| | TATCTTACTGGACGGAGAGATATTCGCCTTCGACTACACCGGCTACGACGC | |
| | CAGCCTGAGCCCTGTGTGGTTCGCCTGCCTGAAGAAGGTGTTGATCAAGCT | |
| | GGGCTACACCCACCAGACCAGCTTCATCGACTACCTGTGCCACTCGGTGCA | |
| | TCTGTACAAGGACAAGAAGTATATCGTCAACGGCGGCATGCCTAGCGGCA | |
| | GCAGCGGCACCTCTATCTTCAATACCATGATCAACAACATCATCATCAGAA | |
| | CCCTGCTCATTAGAGTCTATAAGGGCATCGACCTGGACCAGTTCAAGATGA | |
| | TCGCCTACGGCGACGACGTAATCGCCAGCTACCCTCACAAGATCGACCCTG | |
| | GCCTGCTGGCCGAGGCCGGCAAGCAGTACGGACTGGTGATGACCCCTGCC | |
| | GACAAGGGAACTTCGTTCATTGATACTAATTGGGAGAACGTCACCTTCCTT | |
| | AAGAGATACTTCAGAGCCGACGACCAGTACCCTTTCCTGATCCACCCTGTG | |
| | ATGCCTATGAAGGAGATCCACGAGAGCATCCGCTGGACCAAGGATCCACG | |
| | CAACACCCAGGACCACGTGAGAAGCCTGTGCTATCTGGCTTGGCACAACG | |
| | GCGAAGAAGCCTATAACGAGTTCTGCAGAAAGATCAGAAGCGTGCCAGTG | |
| | GGCAGGGCCCTGACCCTGCCTGCCTACTCGTCGTTGAGAAGAAAGTGGTTG | |
| | GACAGCTTC | |
| 3′ UTR | TGATAATAGGCTGGAGCCTCGGTGGCCTAGCTTCTTGCCCCTTGGGCCTCC | 4 |
| | CCCCAGCCCCTCCTCCCCTTCCTGCACCCGTACCCCCGTGGTCTTTGAATAA | |
| | AGTCTGAGTGGGCGGC | |
| Corresponding | MGPGFDFAQAIMKKNTVIARTEKGEFTMLGVYDRVAVIPTHASVGETIYINDV | 20 |
| Amino Acid | ETKVLDACALRDLTDTNLEITIVKLDRNQKFRDIRHFLPRYEDDYNDAVLSVH | |
| Sequence | TSKFPNMYIPVGQVTNYGFLNLGGTPTHRILMYNFPTRAGQCGGVVTTTGKVI | |
| | GIHVGGNGAQGFAAMLLHSYFTDTQGEIVSSEKSGVCINAPAKTKLQPSVFHQ | |
| | VFEGSKEPAVLNPKDPRLKTDFEEAIFSKYTGNKIMLMDEYMEEAVDHYVGC | |
| | LEPLDISVDPIPLESAMYGMDGLEALDLTTSAGFPYLLQGKKKRDIFNRHTRDT | |
| | SEMTKMLEKYGVDLPFVTFVKDELRSREKVEKGKSRLIEASSLNDSVAMRVA | |
| | FGNLYATFHNNPGTATGSAVGCDPDIFWSKIPILLDGEIFAFDYTGYDASLSPV | |
| | WFACLKKVLIKLGYTHQTSFIDYLCHSVHLYKDKKYIVNGGMPSGSSGTSIFN | |
| | TMINNITIRTLLIRVYKGIDLDQFKMIAYGDDVIASYPHKIDPGLLAEAGKQYGL | |
| | VMTPADKGTSFIDTNWENVTFLKRYFRADDQYPFLIHPVMPMKEIHESIRWTK | |
| | DPRNTQDHVRSLCYLAWHNGEEAYNEFCRKIRSVPVGRALTLPAYSSLRRKW | |
| | LDSF | |
| Poly A tail | 100 nt | |

EV-A71 C4a subgenotype consensus P1

| Chemistry: 1-methylpseudouridine | | |
|---|---|---|
| Cap: 7mG(5′)ppp(5′)NlmpNp | | |
| SEQ ID NO: 21 consists of from 5′ end to 3′ end: 5′ UTR SEQ ID NO: 2, | | 21 |
| mRNA ORF SEQ ID NO: 22, and 3′ UTR SEQ ID NO: 4. | | |
| 5′ UTR | GGGAAATAAGAGAGAAAAGAAGAGTAAGAAGAAATATAAGACCCCGGCG | 2 |
| | CCGCCACC | |
| ORF of | ATGGGAAGTCAGGTGTCCACTCAAAGAAGTGGCAGCCACGAGAACAGCAA | 22 |
| mRNA | CAGCGCCACCGAGGGCAGCACCATCAACTACACAACAATCAACTACTACA | |
| Construct | AAGACAGCTACGCCGCTACCGCGGGCAAGCAGAGCCTGAAGCAGGATCCG | |
| (excluding | GACAAGTTCGCTAATCCTGTGAAGGACATCTTCACCGAGATGGCCGCGCCA | |
| stop codon) | CTGAAGTCTCCTAGCGCCGAGGCCTGCGGCTACAGCGACAGGGTGGCCCA | |
| | GCTGACCATAGGGAACAGCACAATCACCACCCAGGAGGCCGCCAACATCA | |
| | TCGTGGGTTACGGCGAGTGGCCTAGCTACTGTAGCGATAGCGACGCCACC | |
| | GCCGTAGATAAGCCAACCAGACCTGACGTGAGCGTTAACCGGTTCTACACT | |
| | CTCGACACTAAGCTTTGGGAGAAGTCTAGCAAAGGTTGGTACTGGAAGTTC | |
| | CCTGACGTGCTGACCGAAACCGGAGTGTTCGGCCAGAACGCCCAGTTCCA | |
| | CTACCTGTATAGAAGCGGCTTTTGCATCCACGTACAGTGCAACGCCTCTAA | |
| | GTTTCACCAGGGCGCACTGCTGGTTGCCGTCCTGCCTGAATACGTTATCGG | |
| | CACGGTTGCCGGCGGTACGGGCACCGAGGACACCCACCCGCCCTACAAGC | |
| | AGACGCAGCCTGGCGCCGACGGCTTCGAGCTTCAGCATCCTTACGTGTTGG | |
| | ACGCCGGGATCCCTATTTCTCAGCTTACCGTGTGCCCTCACCAGTGGATCA | |
| | ACCTGAGAACCAATAACTGCGCGACAATCATAGTACCTTACATCAACGCCT | |
| | TGCCTTTCGACAGCGCCCTGAATCACTGCAACTTCGGCCTGCTCGTGGTGC | |
| | CTATCTCCCCTCTTGATTACGACCAGGGTGCCACCCCTGTGATCCCCATTAC | |
| | CATCACCCTGGCACCTATGTGTAGCGAGTTCGCCGGCCTCAGGCAGGCCGT | |
| | GACCCAGGGCTTCCCAACCGAGCTGAAGCCGGGCACCAACCAATTCCTCA | |
| | CTACCGACGACGGCGTGAGCGCCCCAATCCTGCCTAACTTCCACCCTACAC | |
| | CGTGCATCCACATTCCTGGCGAGGTTAGAAACCTGCTGGAGCTTTGTCAGG | |
| | TGGAGACTATTCTGGAAGTGAATAACGTGCCTACCAACGCCACAAGTCTG | |
| | ATGGAGAGACTGCGCTTCCCTGTGAGCGCGCAAGCCGGGAAGGGCGAGCT | |
| | GTGCGCCGTGTTCCGAGCCGACCCTGGCAGAAACGGCCCGTGGCAAAGCA | |
| | CTCTGCTGGGCCAGTTGTGCGGCTACTACACCCAGTGGTCCGGCAGCCTCG | |
| | AGGTCACCTTCATGTTCACCGGAAGCTTCATGGCCACCGGCAAGATGCTGA | |
| | TCGCCTACACCCCTCCTGGAGGCCCTCTGCCTAAGGACAGAGCCACTGCCA | |
| | TGCTGGGGACCCACGTGATCTGGGACTTCGGACTGCAGTCCAGCGTGACTC | |
| | TGGTCATCCCTTGGATCTCCAATACCCACTATAGAGCTCACGCCAGAGACG | |
| | GTGTGTTCGACTATTACACCACAGGCCTGGTTTCCATCTGGTACCAGACCA | |
| | ATTACGTGGTGCCCATCGGCGCCCCTAACACCGCCTATATCATCGCCCTGG | |
| | CAGCTGCCCAGAAGAACTTCACCATGAAACTGTGCAAGGACGCATCAGAC | |
| | ATCCTGCAGACCGGCACGATCCAGGGCGACCGAGTGGCCGACGTGATCGA | |
| | GAGTTCAATCGGCGACAGCGTGAGCCGGGCCCTGACCCACGCACTGCCCG | |
| | CCCCTACAGGTCAGAACACCCAGGTCAGCAGCCACCGTCTGGATACAGGC | |
| | AAAGTGCCTGCGCTGCAGGCCGCAGAGATTGGAGCCAGCTCAAACGCCTC | |
| | CGACGAGTCAATGATCGAAACTAGGTGCGTGCTGAACAGCCATAGCACAG | |
| | CCGAAACCACCCTGGACAGCTTCTTCTCTCGCGCAGGACTGGTCGGCGAGA | |
| | TAGACCTGCCTCTGGAGGGCACCACCAACCCTAACGGCTACGCGAACTGG | |
| | GACATTGACATCACCGGCTACGCCCAAATGAGGAGAAAGGTGGAACTGTT | |
| | CACTTACATGAGGTTTGACGCCGAATTCACTTTCGTGGCGTGCACCCCGAC | |
| | CGGAGAGGTGGTGCCTCAGCTGCTGCAGTATATGTTTGTTCCACCTGGCGC | |
| | ACCAAAGCCTGATAGCAGAGAGAGCCTGGCCTGGCAGACCGCAACCAATC | |
| | CTAGTGTGTTCGTGAAGCTGAGCGATCCTCCAGCCCAGGTGAGCGTGCCTT | |
| | TCATGAGCCCTGCTAGCGCCTACCAGTGGTTTTACGACGGGTACCCTACCT | |
| | TCGGCGAGCACAAGCAGGAGAAGGACTTGGAGTACGGCGCCTGCCCGAAC | |
| | AACATGATGGGCACCTTCAGTGTGCGGACGGTCGGAACTAGCAAGAGCAA | |
| | GTATCCTCTGGTGGTGAGGATTTACATGCGCATGAAGCACGTCCGTGCGTG | |
| | GATCCCTAGACCTATGCGTAACCAGAACTACCTGTTCAAGGCCAATCCGAA | |
| | TTACGCCGGCAATTCCATCAAGCCTACGGGTGCGAGCCGCACCGCGATTAC | |
| | CACACTG | |
| 3′ UTR | TGATAATAGGCTGGAGCCTCGGTGGCCTAGCTTCTTGCCCCTTGGGCCTCC | 4 |
| | CCCCAGCCCCTCCTCCCCTTCCTGCACCCGTACCCCCGTGGTCTTTGAATAA | |
| | AGTCTGAGTGGGCGGC | |
| Corresponding | MGSQVSTQRSGSHENSNSATEGSTINYTTINYYKDSYAATAGKQSLKQDPDKF | 23 |
| Amino Acid | ANPVKDIFTEMAAPLKSPSAEACGYSDRVAQLTIGNSTITTQEAANIIVGYGEW | |
| Sequence | PSYCSDSDATAVDKPTRPDVSVNRFYTLDTKLWEKSSKGWYWKFPDVLTETG | |
| | VFGQNAQFHYLYRSGFCIHVQCNASKFHQGALLVAVLPEYVIGTVAGGTGTE | |
| | DTHPPYKQTQPGADGFELQHPYVLDAGIPISQLTVCPHQWINLRTNNCATIIVP | |
| | YINALPFDSALNHCNFGLLVVPISPLDYDQGATPVIPITITLAPMCSEFAGLRQA | |
| | VTQGFPTELKPGTNQFLTTDDGVSAPILPNFHPTPCIHIPGEVRNLLELCQVETIL | |
| | EVNNVPTNATSLMERLRFPVSAQAGKGELCAVFRADPGRNGPWQSTLLGQLC | |
| | GYYTQWSGSLEVTFMFTGSFMATGKMLIAYTPPGGPLPKDRATAMLGTHVIW | |
| | DFGLQSSVTLVIPWISNTHYRAHARDGVFDYYTTGLVSIWYQTNYVVPIGAPN | |
| | TAYIIALAAAQKNFTMKLCKDASDILQTGTIQGDRVADVIESSIGDSVSRALTH | |
| | ALPAPTGQNTQVSSHRLDTGKVPALQAAEIGASSNASDESMIETRCVLNSHST | |
| | AETTLDSFFSRAGLVGEIDLPLEGTTNPNGYANWDIDITGYAQMRRKVELFTY | |
| | MRFDAEFTFVACTPTGEVVPQLLQYMFVPPGAPKPDSRESLAWQTATNPSVF | |
| | VKLSDPPAQVSVPFMSPASAYQWFYDGYPTFGEHKQEKDLEYGACPNNMMG | |
| | TFSVRTVGTSKSKYPLVVRIYMRMKHVRAWIPRPMRNQNYLFKANPNYAGN | |
| | SIKPTGASRTAITTL | |
| Poly A tail | 100 nt | |

EV-A71 C4 subgenotype 3CD protease

| Chemistry: 1-methylpseudouridine | | |
|---|---|---|
| Cap: 7mG(5′)ppp(5′)NlmpNp | | |
| SEQ ID NO: 24 consists of from 5′ end to 3′ end: 5′ UTR SEQ ID NO: 2, | | 24 |
| mRNA ORF SEQ ID NO: 25, and 3′ UTR SEQ ID NO: 4. | | |
| 5′ UTR | GGGAAATAAGAGAGAAAAGAAGAGTAAGAAGAAATATAAGACCCCGGCG | 2 |
| | CCGCCACC | |
| ORF of | ATGGGCCCTAGCCTGGACTTCGCCCTGAGCCTGCTGAGAAGAAACATCAG | 25 |
| mRNA | ACAGGTGCAGACCGACCAGGGCCACTTCACCATGCTGGGCGTGAGAGACA | |
| Construct | GACTGGCCGTGCTGCCTAGACACAGCCAGCCTGGCAAGACCATCTGGATC | |
| (excluding | GAGCACAAGCTGGTGAACGTGCTGGACGCCGTGGAGCTGGTGGACGAGCA | |
| stop codon) | GGGCGTGAACCTGGAGCTGACCCTGATCACCCTGGACACCAACGAGAAGT | |
| | TCAGAGACATCACCAAGTTCATCCCTGAGAACATCAGCACCGCCAGCGAC | |
| | GCCACCCTGGTGATCAACACCGAGCACATGCCTAGCATGTTCGTGCCTGTG | |
| | GGCGACGTGGTGCAGTACGGCTTCCTGAACCTGAGCGGCAAGCCTACCCA | |
| | CAGAACCATGATGTACAACTTCCCTACCAAGGCTGGACAGTGTGGCGGCG | |
| | TGGTGACCAGCGTGGGCAAGGTGATCGGCATCCACATCGGCGGCAACGGC | |
| | AGACAGGGCTTCTGCGCCGGCCTGAAGAGAAGCTACTTCGCCTCTGAACA | |
| | GGGTGAGATCCAGTGGGTGAAGCCTAACAAGGAGACTGGCAGACTGAACA | |
| | TCAACGGCCCTACCAGAACCAAGCTGGAGCCTAGCGTGTTCCACGACATCT | |
| | TCGAGGGCAATAAGGAGCCAGCCGTCCTGCACAGCAAGGACCCTAGACTG | |
| | GAGGTGGATTTCGAGCAGGCCCTGTTCAGCAAGTACGTCGGCAACACCCT | |
| | GCACGAGCCTGACGAGTACATCAAGGAGGCCGCCCTGCACTACGCCAACC | |
| | AGCTGAAGCAGCTGGAGATCAATACGAGCCAGATGAGCATGGAGGAGGCC | |
| | TGCTACGGCACCGAGAACCTCGAGGCCATCGACCTGCACACCAGCGCCGG | |
| | CTACCCTTACAGCGCCCTGGGCATCAAGAAGCGGGACATCCTGGACCCTAC | |
| | CACCAGAGACGTGAGCAAGATGAAGTTCTACATGGACAAGTACGGCCTGG | |
| | ACCTGCCATACAGCACCTACGTGAAGGACGAGCTCAGGAGCATCGACAAG | |
| | ATTAAGAAGGGCAAGAGTAGACTGATAGAAGCCAGCAGCCTGAACGACA | |
| | GCGTGTACCTGAGAATGGCCTTCGGCCACCTGTACGAGGCCTTCCACGCTA | |
| | ATCCTGGCACCATCACCGGCAGCGCCGTGGGCTGCAACCCTGACACCTTCT | |
| | GGAGCAAGCTGCCTATCCTGCTGCCTGGCAGCCTGTTCGCCTTCGACTACA | |
| | GCGGCTACGACGCTTCTCTTAGCCCTGTGTGGTTCAGAGCACTCGAACTGG | |
| | TGCTGAGAGAGATCGGCTACAGCGAGGGCGCCGTGAGCCTCATTGAGGGT | |
| | ATCAACCACACCCACCACGTGTACAGAAACAAGACCTACTGCGTGTTGGG | |
| | AGGAATGCCGAGTGGCTGCAGCGGCACCAGCATCTTCAACAGCATGATTA | |
| | ATAACATCATCATACGGGCCCTACTGATCAAGACCTTCAAGGGCATTGACC | |
| | TGGACGAGTTAAACATGGTGGCCTACGGCGACGACGTGCTCGCCAGCTAC | |
| | CCGTTCCCTATCGACTGTCTTGAGCTCGCCAAGAACGGCAAGGAATACGGC | |
| | TTGACCATGACCCCTGCCGACAAGTTCCCTTGCTTCAAGGAAGTGAACTGG | |
| | GGCAACGCCACGTTCCTGAAGCGTGGTTTCCTTCCAGACGAACAGTTCCCA | |
| | TTCCTGATCCCTCCTACCATGCCTATGCGCGAAATCCACGAGAGCATCCGC | |
| | TGGACCAAGGACGCCAGAAACCCTCAGGACCACGTGAGAAGCCTGTGCCT | |
| | GCTGGCCTGGCACAACGGCAAGCAGGAGTACGAGAAGTTCGTGAGCACGA | |
| | TAAGAAGCGTGCCGGTAGGCAGGGCACTGGCCATCCCTAACTACGAGAAT | |
| | CTCAGGAGGAATTGGCTGGAACTGTTC | |
| 3′ UTR | TGATAATAGGCTGGAGCCTCGGTGGCCTAGCTTCTTGCCCCTTGGGCCTCC | 4 |
| | CCCCAGCCCCTCCTCCCCTTCCTGCACCCGTACCCCCGTGGTCTTTGAATAA | |
| | AGTCTGAGTGGGCGGC | |
| Corresponding | MGPSLDFALSLLRRNIRQVQTDQGHFTMLGVRDRLAVLPRHSQPGKTIWIEHK | 26 |
| Amino Acid | LVNVLDAVELVDEQGVNLELTLITLDTNEKFRDITKFIPENISTASDATLVINTE | |
| Sequence | HMPSMFVPVGDVVQYGFLNLSGKPTHRTMMYNFPTKAGQCGGVVTSVGKVI | |
| | GIHIGGNGRQGFCAGLKRSYFASEQGEIQWVKPNKETGRLNINGPTRTKLEPS | |
| | VFHDIFEGNKEPAVLHSKDPRLEVDFEQALFSKYVGNTLHEPDEYIKEAALHY | |
| | ANQLKQLEINTSQMSMEEACYGTENLEAIDLHTSAGYPYSALGIKKRDILDPTT | |
| | RDVSKMKFYMDKYGLDLPYSTYVKDELRSIDKIKKGKSRLIEASSLNDSVYLR | |
| | MAFGHLYEAFHANPGTITGSAVGCNPDTFWSKLPILLPGSLFAFDYSGYDASL | |
| | SPVWFRALELVLREIGYSEGAVSLIEGINHTHHVYRNKTYCVLGGMPSGCSGT | |
| | SIFNSMINNIIIRALLIKTFKGIDLDELNMVAYGDDVLASYPFPIDCLELAKNGK | |
| | EYGLTMTPADKFPCFKEVNWGNATFLKRGFLPDEQFPFLIPPTMPMREIHESIR | |
| | WTKDARNPQDHVRSLCLLAWHINGKQEYEKFVSTIRSVPVGRALAIPNYENLR | |
| | RNWLELF | |
| Poly A tail | 100 nt | |

### Additional Materials & Methods

### Transfections & Immunoblotting

293T cells were transfected with mRNA using the Mirus Trans-It transfection kit. Cells were transfected with 2 jig of P1 mRNA and indicated amounts of 3CD mRNA. Cells were harvested ˜16-24 h post-transfection and lysed with RIPA buffer. Lysates were quantified for protein concentration by BCA assay, run on 4-20% polyacrylamide gels with reducing agent (25 gig/lane), transferred to nitrocellulose membranes, and blotted overnight with primary antibodies at a 1 μg/ml dilution. Primary antibodies used were mouse anti-HRV-A16 VP2 and rabbit anti-pan HRV VP1. HRP-conjugated anti-mouse and anti-rabbit secondary antibodies were used at a 1:10,000 dilution. Signal was detected using ECL Prime detection reagent. A rhodamine-conjugated anti-GAPDH Fab was used for GAPDH detection.

### Virus-Like Particle Production, Purification & Imaging

Plasmids encoding HRV-A16 P1 and 3CD are co-transfected into HEK-293expi cells at 3e6/mL density using Turbo293 transfection reagent (Speed Biosystems), with a 4:1 mass ratio between P1 and 3CD plasmids. Six days later, cell culture supernatant is harvested (cell pellet discarded) before adding PEG-6000 and sodium chloride to final concentrations of 7% (w/v) and 2% (w/v), respectively. PEG (polyethylene glycol) precipitation is carried out for 18 hours at 4° C. with agitation, followed by centrifugation at 9,500K at 4° C. for 30 minutes. The supernatant is discarded, and the precipitation resuspended in 50 mM phosphate buffer, pH 7.0 (with no sodium chloride). The resuspended solution is passed through a 0.45 um filter to removed large debris. The sample is then loaded onto a HiTrap Q (Cytiva) column on a Akta Pure (Cytiva) for anion exchange chromatography. HRV VLP bound to the HiTrap Q column is first washed with 0.1M sodium chloride in phosphate buffer pH 7.0 to remove impurity, followed by elution using 0.2M sodium chloride in phosphate buffer pH 7.0. The eluted HRV VLP samples are used to prepare electron microscope grids with nano-W negative stain (Nanoprobes) and imaged using a Tecnai T12 electron microscope.

### Vaccination and In Vivo Studies

Approximately 16 h prior to vaccination, formulated P1 and 3CD products were mixed and diluted with PBS to achieve the desired P1:3CD ratio and final mRNA concentration. Female 8 wk-old Balb/c mice were vaccinated (50 μl injection volume, IM) on Day 1 and Day 22. Serum was collected 3 weeks post-first injection (Day 21) and 2 weeks post-second injection (Day 35). Mice were sacrificed following the Day 35 serum collection.

### Neutralization Assays

Virus isolates and titration: Viruses HRV-A16; HRV-B14; EV-D68 isolate US/MO/14-18947; EV-D68 isolate US/KY/14-18953 were purchased from ATCC. EV-A71 isolates USA/2018-23092 and Tainan/4643/1998, were received from BEI Resources. Viral titer was measured by endpoint dilution and calculated using the Reed-Muench statistical method. HRV-A16 and HRV-B14 stocks were titered on MRC-5 cells, and EV-D68 and EV-A71 isolate stocks titered on RD cells. The TCID₅₀ of each virus stock was calculated by scoring cells for cytopathic effect (CPE) via microscope five days post-infection.

Rhinovirus neutralization assays: MRC-5 cells were plated in 48-well plates at 2×10⁴ cells/well, and incubated in MEM until wells reached ˜90% confluency (usually seven days after plating). Serum from vaccinated mice was incubated at 56° C. for 30 minutes to inactivate complement, then diluted four-fold in a series of six dilutions. Diluted serum was mixed 1:2 with 100 TCID₅₀ virus to give a final volume of 100 μl serum+virus. To allow neutralization, serum+virus was incubated at 33° C. for 3 h, during which time the media was aspirated from 48-well plates and replaced with 300 μl fresh MEM. After 3 h neutralization, serum+virus was added to plates. Final volume was 400 μl/well. Plates were placed at 33° C. After five days, plates were removed and wells scored for CPE via microscope. Each dilution contained eight replicate wells. The neutralizing antibody titer (reported as IC₅₀) was calculated using the Reed-Muench method.

EV-D68 and EV-A71 neutralization assays: For EV-D68 neutralization. R) cells were plated in 96-well plates at 10⁴ cells/well in 100 μl DMEM and used in the neutralization assay 48 hours post-plating. For EV-A71 neutralization, Vero cells were plated in 96-well plates at 1.5×10⁴ cells/well in 100 μl DMEM and used in the neutralization assay the following day. RD cells were plated in 96-well plates at 10⁴ cells/well in 100 μl DMEM. Forty-eight hours post-plating, heat-inactivated serum from vaccinated mice was diluted four-fold in a series of six dilutions. Diluted serum was mixed 1:2 with 100 TCID₅₀ virus to give a final volume of 100 μl serum+virus. To allow neutralization, serum+virus was incubated for 3 h at 33° C. for EV-D68 and 37° C. for EV-A71. After 3 h incubation, serum+virus was added to plates. Final volume/well was 200 μl. Only inner 60 wells were used to avoid edge effects. Outer 36 wells were brought up to 200 μl final volume with DMEM. EV-D68 neutralization plates were placed at 33° C., and EV-A71 neutralization plates were placed at 37° C. After five days, plates were removed and wells scored for CPE via microscope. Each dilution contained ten replicate wells. The neutralizing antibody titer (reported as IC₅₀) was calculated using the Reed-Muench method. Antiserum: Guinea pig hyperimmune antiserum for HRV-A16 and HRV-B14 was purchased from ATCC. Cotton rat HRV-A16 antiserum was generated by injecting 10 female cotton rats with 10s PFU replicative HRV-A16 on Days 1 and 22 and collecting and pooling serum from vaccinated animals on Day 35. Antiserum was heat-treated at 56° C. for 30 minutes to inactivate complement. Antiserum was diluted and run in neutralization assays as described above.

### Cotton Rat HRV-A16 Challenge Study

Animals: Forty (36) inbred, 4 to 6 weeks-old, Sigmodon hispidus female cotton rats (source: Sigmovir Biosystems, Inc., Rockville MD) were maintained and handled under veterinary supervision in accordance with the National Institutes of Health guidelines and Sigmovir Institutional Animal Care and Use Committee's approved animal study protocol (IACUC Protocol #2). Cotton rats were housed in clear polycarbonate cages and provided with standard rodent chow (Harlan #7004) and tap water ad lib.

Challenge Virus: Human rhinovirus type 16 (HRV16) (ATCC, Manassas, VA) was propagated in HeLa Ohio cells after serial plaque-purification to reduce defective-interfering particles. Virus stock was chloroform extracted for further purification. A pool of virus designated as HRV16 p5 containing approximately 1.0×10⁸ pfu/mL was used for this in vivo experiment. This stock of virus is stored under −80° C. conditions and has been characterized in vivo in the cotton rat model and validated for the upper and lower respiratory tract replication.

Lung and Nose HRV16 viral titration: Lung and nose homogenates are clarified by centrifugation and diluted (lung, 1/10-1/100 dilutions; nose, Neat-1/10 dilutions) in infection media (EMEM, Glutamine, Pyrubate, Na-Bicarbonate, Hepes). Confluent Hela H1 monolayers are infected in duplicates with 100 μl per well starting with the first dilution followed by diluted homogenates in 6-well plates. After 1 h incubation at 33° C. in a 5% CO₂ incubator, wells are overlaid with 0.75% methylcellulose medium and plates restored into the 33° C. incubator. After 5 days of incubation the overlay is removed and the cells are fixed with 0.1% crystal violet stain for one hour, then rinsed, and air-dried. Plaques are counted and virus titers are expressed as plaque forming units per gram of tissue. Viral titers in a group are calculated as the geometric mean t standard error for all animals in that group at a given time.

HRV16 neutralizing antibody assay: Serum samples are diluted 1:10 with infection media and serially diluted further 1:2. Diluted serum samples are incubated with HRV16 (10′ TCID₅₀) for 2 h at 33° C. in a 5% CO₂ incubator. Virus-antibody complexes are transferred to confluent Hela H1 cells in 96 well plates. After 5 days of incubation, cells are fixed and stained with 0.1% crystal violet for 1 h and then rinsed and air dried. Neutralizing antibody titers are determined by the Reed and Muench method by determining the cut off dilution that fully protect the cell monolayer.

Real-time PCR viral RNA: Total RNA is extracted from homogenized lung tissue using the RNeasy purification kit (QIAGEN). One μg of total RNA is used to prepare cDNA using QuantiTect Reverse Transcription Kit (Qiagen). For the real-time PCR reactions, the QuantiFast SYBR Green PCR Kit (Qiagen) is used in a final volume of 25 μl, with final primer concentrations of 0.5 μM. Reactions are set up in 96-well trays. Amplifications are performed on a Bio-Rad iCycler for 1 cycle of 95° C. for 3 min, followed by 40 cycles of 95° C. for 10 sec, 60° C. for 10 sec. and 72° C. for 15 sec. The baseline cycles and cycle threshold (Ct) are calculated by the iQ5 software in the PCR Base Line Subtracted Curve Fit mode. Relative quantification of DNA is applied to all samples. The standard curves are developed using serially-diluted cDNA sample most enriched in the transcript of interest (e.g., lungs from day 8 h HRV16 infection for viral transcripts qPCR). The Ct values are plotted against log₁₀ cDNA dilution factor. These curves are used to convert the Ct values obtained for different samples to relative expression units. These relative expression units are then normalized to the level of β-actin mRNA (“housekeeping gene”) expressed in the corresponding sample.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited. In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. A composition comprising:
(i) a messenger ribonucleic acid (mRNA) comprising an open reading frame (ORF) encoding a picornavirus capsid polyprotein, wherein the capsid polyprotein comprises a viral P1 precursor polyprotein; and
(ii) a lipid nanoparticle (LNP).

2. A composition comprising:
(i) a messenger ribonucleic acid (mRNA) comprising an open reading frame (ORF) encoding a protease, wherein the protease is a picornavirus 3C protease; and
(ii) a lipid nanoparticle (LNP).

3. An immunogenic composition comprising:
(i) a messenger ribonucleic acid (mRNA) comprising an open reading frame (ORF) encoding a picornavirus 3C protease;
(ii) an mRNA comprising an ORF encoding a picornavirus capsid polyprotein, wherein the capsid polyprotein comprises a viral P1 precursor polyprotein; and
(iii) a lipid nanoparticle (LNP).

4. The composition of claim 1 or 3, wherein the precursor polyprotein comprises two or more capsid proteins and has a cleavage site specific for a viral protease between the two or more capsid proteins.

5. The composition of claim 4, wherein the two or more capsid proteins comprise two or more of viral protein 0 (VP0), viral protein 1 (VP1), and viral protein 3 (VP3).

6. The composition of claim 5, wherein VP0 further comprises viral protein 2 (VP2) and viral protein 4 (VP4), and wherein VP2 and VP4 comprise a cleavage site for capsid maturation.

7. The composition of any one of claims 2-4 and 6, wherein the protease is Picornavirus 3C (3CD).

8. The composition of claim 7, wherein the 3CD is specific to a species in the genus Enterovirus in the picornavirus family.

9. The composition of claim 7 or 8, wherein the 3CD is specific to a species of human rhinovirus (HRV) A, B or C.

10. The composition of claim 9, wherein the HRV species is HRV-A16.

11. The composition of claim 9, wherein the HRV species is HRV-B14.

12. The composition of claim 7 or 8, wherein the 3CD is specific to an Enterovirus species.

13. The composition of claim 12, wherein the Enterovirus genotype is EV-D68.

14. The composition of claim 12, wherein the Enterovirus genotype is EV-A71.

15. The composition of any one of claims 1 and 3-14, wherein the capsid proteins form a protomer.

16. The composition of claim 15, wherein the protomers form a pentamer.

17. The composition of claim 16, wherein the pentamers form a virus-like particle (VLP).

18. The composition of claim 3-17, wherein mRNA comprising the ORF encoding the viral P1 precursor polyprotein and the mRNA comprising the ORF encoding the picornavirus 3C protease (P1:3CD) are present in one of the following ratios: 20:1, 10:1, 7:1, 5:1, 4:1, 3:1, 2:1, 1:1.

19. The composition of claim 18, wherein the ratio of mRNA comprising the ORF encoding the viral P1 precursor protein and the mRNA comprising the ORF encoding the picornavirus 3C protease is 10:1.

20. The composition of claim 18, wherein the ratio of mRNA comprising the ORF encoding the viral P1 precursor polyprotein and the mRNA comprising the ORF encoding 3C protease as either 3C or 3CD is 2:1.

21. The composition of any one of claims 3-20, wherein the protease comprises an amino acid sequence having at least 90% identity to the amino acid sequence of SEQ ID NO: 8 or SEQ ID NO: 14.

22. The composition of claim 21, wherein the protease comprises the amino acid sequence of SEQ ID NO: 8 or SEQ ID NO: 14.

23. The composition of any one of claims 3-20, wherein the protease comprises an amino acid sequence having at least 90% identity to the amino acid sequence of SEQ ID NO: 20 or SEQ ID NO: 26.

24. The composition of claim 23, wherein the protease comprises the amino acid sequence of SEQ ID NO: 20 or SEQ ID NO: 26.

25. The composition of any one of claims 1, and 3-24, wherein the capsid polyprotein comprises an amino acid sequence having at least 90°/% identity to the amino acid sequence of any one of SEQ ID NOs: 5, 11, 17, or 23.

26. The composition of claim 25, wherein the capsid polyprotein comprises the amino acid sequence of any one of SEQ ID NOs: 5, 11, 17, or 23.

27. The composition of any one of claims 17-26, wherein the VLP comprises Neutralizing Immunogenic (NIm) sites.

28. The composition of any one of claims 1, 2, or 4-27, wherein the LNP comprises an ionizable amino lipid, a PEG-modified lipid, a structural lipid and a phospholipid.

29. The composition of any one of claims 3-27, wherein the mRNA comprising the ORF encoding the viral P1 precursor polyprotein and the mRNA comprising the ORF encoding the picornavirus 3C protease are co-formulated in at least one LNP.

30. The composition of any one of claims 3-27, wherein the mRNA comprising the ORF encoding the viral P1 precursor polyprotein and the mRNA comprising the ORF encoding the picornavirus 3C protease are each formulated in separate LNPs.

31. The composition of claim 29, wherein the LNP formulated with mRNA comprising the ORF encoding the viral P1 precursor polyprotein and the mRNA comprising the ORF encoding the picornavirus 3C protease are present in one of the following ratios: 20:1, 10:1, 7:1, 5:1, 4:1, 3:1, 2:1, 1:1.

32. The composition of any one of claims 29-31, wherein the LNP comprises an ionizable amino lipid, a sterol, neutral lipid, and a PEG-modified lipid.

33. The composition of claim 32, wherein the lipid nanoparticle comprises 40-55 mol % ionizable amino lipid, 30-45 mol % sterol, 5-15 mol % neutral lipid, and 1-5 mol % PEG modified lipid.

34. The composition of claim 33, wherein the lipid nanoparticle comprises 40-50 mol % ionizable amino lipid, 35-45 mol % sterol, 10-15 mol % neutral lipid, and 2-4 mol % PEG-modified lipid.

35. The composition of any one of the preceding claims, wherein the lipid nanoparticle comprises 45 mol %, 46 mol %, 47 mol %, 48 mol %, 49 mol %, or 50 mol % ionizable amino lipid.

36. The composition of any one of the preceding claims, wherein the ionizable amino lipid has the structure of Compound 2:

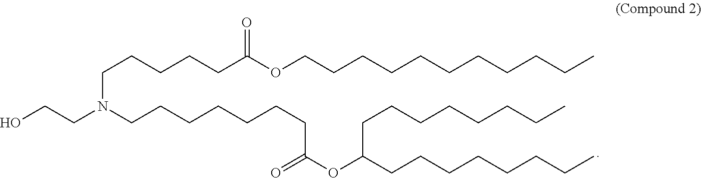 [SMILES: CCCCCCCCCCCOC(=O)CCCCCN(CCO)CCCCCCCC(=O)OC(CCCCCCCC)CCCCCCCC]

37. The composition of any one of claims 27-31, wherein the sterol is cholesterol or a variant thereof.

38. The composition of any one of claims 27-37, wherein the neutral lipid is 1,2 distearoyl-sn-glycero-3-phosphocholine (DSPC).

39. A method comprising administering to a subject an immunogenic composition comprising:
(i) a messenger ribonucleic acid (mRNA) comprising an open reading frame encoding a picornavirus protease; and
(ii) a messenger ribonucleic acid (mRNA) comprising an open reading frame encoding a capsid polyprotein comprising a precursor protein, wherein the precursor protein comprises two or more capsid proteins and has a cleavage site specific for the protease between the two or more capsid proteins,
in an amount effective to induce in the subject an immune response against a viral infection from a member of the Enterovirus genus.

40. The method of claim 39, wherein the immune response includes a binding antibody titer to a human rhinovirus species of the Enterovirus genus.

41. The method of claim 39, wherein the immune response includes a neutralizing antibody titer to a human rhinovirus species of the Enterovirus genus.

42. The method of claim 39, wherein the immune response includes a T cell response to a human rhinovirus species of the Enterovirus genus.

43. The method of claims 40-42, wherein the human rhinovirus species of virus is selected from the group consisting of genotypes A2, A16, B14, and C15.

44. The method of claim 41, wherein the human rhinovirus genotype is human rhinovirus 16 (HRV16).

45. The method of claim 39, wherein the immune response includes a binding antibody titer to a human enterovirus species of the Enterovirus genus.

46. The method of claim 39, wherein the immune response includes a neutralizing antibody titer to a human enterovirus species of the Enterovirus genus.

47. The method of claim 39, wherein the immune response includes a T cell response to a human enterovirus species of the Enterovirus genus.

48. The method of claims 45-47, wherein the human enterovirus species of virus is selected from the group consisting of RV-A, RV-B, RV-C, EV-A and EV-D.

49. The method of claim 48, wherein the human enterovirus species of virus is selected from the group consisting of genotypes RV-A16, RV-B14, RV-C15, EV-A71 and EV-D68.

50. The method of claim 49, wherein the human enterovirus species of virus is Enterovirus D68 (EV-D68).

51. The method of claim 49, wherein the human enterovirus species of virus is Enterovirus A71 (EV-A71).

52. The method of claim 39, wherein the mRNA of (i) are formulated in a composition comprising at least one lipid nanoparticle.

53. The method of claim 39 or 52, wherein the mRNA of (ii) are formulated in a composition comprising at least one lipid nanoparticle.

54. The method of claim 53, wherein the mRNA of (i) are administered to the subject at the same time as the mRNA of (ii).

55. The method of claim 39, wherein the mRNA of (i) and (ii) are formulated in a composition comprising at least one lipid nanoparticle.

56. The method of claim 55, wherein the mRNA of (i) and (ii) are formulated in a composition comprising two lipid nanoparticles.

57. A composition comprising:
(i) a first messenger ribonucleic acid (mRNA) comprising an open reading frame (ORF) encoding a first product;
(ii) a second mRNA comprising an ORF encoding a second product; and
(iii) a lipid nanoparticle (LNP),
wherein the first product is an active protein and wherein the active protein can modulate the expression, structure, or activity of the second mRNA and/or the second product.

58. The composition of claim 57, wherein the composition is an immunogenic composition.

59. The composition of claim 57 or 58, wherein the first product is a catalytic protein.

60. The composition of claim 57 or 58, wherein the first product is an enzymatic protein.

61. The composition of claim 57 or 58, wherein the first product is a binding protein.

62. The composition of claim 57 or 58, wherein the first product is a polyprotein.

63. The composition of claim 57 or 58, wherein the second product is a substrate.

64. The composition of claim 57 or 58, wherein the first product is a polyprotein.
